(19) United States
(12) Patent Application Publication
(10) Pub. No.: US 2020/0383985 A1
(43) Pub. Date: Dec. 10, 2020

(54) Inhibitors of Protease Activated Receptor-2

(71) Applicant: Endosome Therapeutics, Inc., Pottstown, PA (US)

(72) Inventors: Luigi Aurelio, Clayton, Victoria (AU); Nigel W. Bunnett, Parkville, Victoria (AU); Bernhard Luke Flynn, Parkville, Victoria (AU); Le Giang, Parkville, Victoria (AU)

(21) Appl. No.: 16/955,581

(22) PCT Filed: Dec. 19, 2018
(86) PCT No.: PCT/JP2018/047988

§ 371 Date: Jun. 18, 2020

(30) Foreign Application Priority Data
Dec. 20, 2017 (AU) 2017905084

(51) Int. Cl. A61K 31/5025; A61P 29/00; A61P 35/00; A61P 25/00; A61K 47/65; C07D 235/02

(52) U.S. Cl. CPC A61K 31/5025; A61P 29/00; C07D 235/02; A61P 25/00; A61K 47/65; A61P 35/00

(57) ABSTRACT

The present invention relates generally to compounds capable of inhibiting Protease Activated Receptor-2 (PAR₂), and uses thereof. More specifically, the present invention relates to inhibitors of PAR₂, to their preparation, and to their use in the treatment of diseases and disorders mediated by PAR₂ signaling.

### FIELD OF THE INVENTION

The present invention relates generally to compounds capable of inhibiting Protease Activated Receptor-2 (PAR₂), and uses thereof. More specifically, the present invention relates to inhibitors of PAR₂, to their preparation, and to their use in the treatment of diseases and disorders mediated by PAR₂ signaling.

### BACKGROUND OF THE INVENTION

Protease-Activated Receptors (PARs), comprising PAR-1, -2, -3, and -4, are a family of G-protein coupled receptors (GPCRs) with a unique mechanism of activation. PARs are not activated directly by endogenous ligands, instead they are activated indirectly by the proteolytic action of enzymes such as thrombin, tissue factors, cathepsin S, tryptase, or trypsin. Typically, proteolytic enzymes cleave a portion from N-termini of PARs, exposing new N-termini that fold back and activate the receptors as endogenous tethered ligands. The specific cleavage sites of PARs are different in the amino acid sequence, and thus are recognized by different enzymes conferring activation selectivity. Thrombin, for example, is the activating enzyme for PAR₁ whereas PAR₂ is activated more readily by trypsin or tryptase. With the exception of PAR₃, short synthetic peptides corresponding to the tethered ligand sequence have been shown to be able to activate the respective PARs.

PAR₂ is widely expressed in various organs, including lung, kidney, heart, liver, skin, smooth muscles and gastrointestinal tract. Presence of PAR₂ has been found in epithelial and endothelial cells and especially in inflammatory cells such as T-cells, monocytes, macrophages, neutrophils, mast cells and eosinophils. A range of host and pathogen-derived serine proteases, including trypsin, mast cell tryptase, tissue kallikreins, members of the coagulation cascade TF-FVIIa and FVa-FXa, cathepsin S, elastase, acrosin, HAT, TMPRSS2, chitinase, bacterial gingipains, Der P1-3, Pen C 13, and testisin can recognize and process the N-terminus of PAR₂. When cleaved at the canonical site (R³⁶S³⁷), the newly exposed N-terminus acts as a tethered ligand inducing activation of PAR₂.

Cleavage at non-canonical sites, for example by cathepsin S, leads to either inactivation of PAR₂ or the unmasking of a different tethered ligand resulting in different signaling profiles. Synthetic peptides mimicking the canonical sequence such as SLIGKV-NH₂ or SLIGRL-NH₂ can selectively activate human PAR₂ with modest potency. Potency of the peptides can be improved by modification of the N-terminal serine (S) residue, a prominent example of which is the potent peptidic agonist 2-fluoryl-LIGRLO-NH₂ (2F agonist).

To date, literature data have shown that activation of PAR₂ is associated with numerous physiological and pathophysiological processes, such as inflammation, tumour metastasis, gastrointestinal motility, pain and itch. PAR₂ activation in monocytes and macrophages has been shown to result in release of inflammatory cytokines and chemokines, such as IL6, IL8, and IL1β (Johansson, U. et al., Journal of Leukocyte Biology, 2005, 78(4): 967-975; Colognato, R. et al., Blood 2003, 102(7): 2645-2652; Steven, R. et al., Innate Immunity 2013, 19(6): 663-672; and Cho, N.-C. et al., Bioorg Med Chem 2015, 23(24): 7717-7727). In addition, administration of PAR₂ agonists in vivo has been shown to elicit inflammatory responses. In particular, a number of research groups have demonstrated that intraplantar administration of PAR₂ activating proteases or synthetic agonists in rodents induces an oedema response and mechanical hyperalgesia that are significantly reduced by treatment with PAR₂ antagonists or by PAR₂ deletion (e.g., Lieu, T. et al., British Journal of Pharmacology 2016, 173(18): 2752-2765). Further studies have also indicated that PAR₂ can function as a mediator of neurogenic inflammation, nociception, and transmission of pain (e.g., Tillu, D. V. et al., Pain 2015, 156(5): 859-867; Zhao, P., et al., Journal of Biological Chemistry 2014, 289(39): 27215-27234). Again, treatment with GB88, a PAR₂ selective antagonist, results in reduction of inflammation and nociceptive actions mediated through PAR₂ (Lieu, T., et al., British Journal of Pharmacology, 2016, 173(18): 2752-2765).

Increased expression of PAR₂ and activating enzymes is implicated in skin disorders such as topic dermatitis (Steinhoff, M. et al., Journal of Neuroscience 2003, 23(15): 6176-6180; Frateschi, S. et al., Nat Commun. 2011, 2: 161). Intralesional application of PAR₂ agonists triggered prolonged ichthyoses, and transgenic expression of PAR₂ provoked epidermal hyperplasia in mouse skin.

PAR₂ expression has been identified in epithelial cells and fibroblasts in the lung, and it is believed to involve in tissue homeostasis via regulation of downstream transcriptional activation (Adams, M. N. et al., Pharmacology & Therapeutics 2011, 130(3): 248-282). Furthermore, several studies have demonstrated that PAR₂ activation promotes cancer cell migration, invasion, and metastasis (e.g., Yau, M-K., L. Liu, and Fairlie, D. P., Journal of Medicinal Chemistry 2013, 56(19): 7477-7497; Zeeh, F. et al., Oncotarget 2016, 7(27): 41095-41109; and Yang, L. et al., Journal of Biological Chemistry 2015, 290(44): 26627-26637).

PAR₁ and PAR₂ have been shown to participate in regulating motility and secretion of the gastrointestinal tract under physiological and pathological conditions. PAR₂ appears to have a dual role since PAR₂ agonists can induce either relaxing or contracting effects depending on the conditions of the experiments. The exact role and mechanism of PAR₂ in regulation of GI motility is still being investigated. However, recent literature data have demonstrated that PAR₂ agonists can stimulate contraction in rodent colon and duodenal muscles (Kawabata, A., M. Matsunami, and F. Sekiguchi, British Journal of Pharmacology 2008, 153: S230-S240; Browning, K. N., Neurogastroenterology and Motility 2010, 22(4): 361-365). In mouse colonic whole muscles trypsin, the endogenous PAR₂ activator elicits biphasic responses: transient hyperpolarization and relaxation followed by repolarization and excitation (Sung, T. S. et al., Journal of Physiology—London 2015, 593(5): 1169-1181).

Results of numerous experiments utilizing PAR₂ deficient mice, inhibition of functions by antibodies or antagonists such as GB88 has revealed a significant role for PAR₂ activation in the pathophysiology of a variety of diseases including diet-induced obesity, adipose inflammation, asthma, rheumatoid arthritis, periodontitis, inflammatory bowel diseases, irritable bowel syndrome, skin diseases, cancer, fibrotic diseases, metabolic dysfunction, chronic pain, and neurological disease (Adams, M. N. et al., Pharmacology & Therapeutics, 2011, 130(3): 248-282).

There is also evidence to suggest that targeting endosomal PAR₂ signaling may offer novel therapeutic methods. There is a growing realization that G protein-coupled receptors (GPCRs), which were formerly considered to function principally at the surface of cells, can continue to signal from endosomes (Murphy, J. E. et al., Proc Natl Acad Sci USA 2009, 106(42): 17615-17622). Although GPCR signaling begins at the plasma membrane, activated receptors associate with β-arrestins (βARRs), which mediate receptor desensitization and endocytosis (DeWire, S. M. et al., Annu Rev Physiol 2007, 69: 483-510). These processes efficiently terminate GPCR signaling at the plasma membrane. The detection of GPCR signaling complexes in endosomes, and the finding that disruption of endocytosis can suppress signaling, suggests that GPCRs signal from endosomes (e.g., May, V. & Parsons, R. L., J Cell Physiol 2017, 232(4): 698-706). GPCRs in endosomes can generate persistent signals in subcellular compartments that control gene transcription and neuronal excitation (Tsvetanova, N. G. & von Zastrow M., Nat Chem Biol 2014, 10(12): 1061-1065). Endosomal signaling of GPCRs has been found to regulate important physiological processes, including pain transmission (Yarwood, R et al., Proc Natl Acad Sci USA 2017, 114(46): 12309-12314).

Proteases and PAR₂ have been implicated in the hypersensitivity of sensory nerves in the colon that may account for chronic pain in patients with irritable bowel syndrome (IBS) (Azpiroz, F. et al., Neurogastroenterol Motil 2007, 19(1 Suppl): 62-88). Biopsies of colonic mucosa from IBS patients release proteases, including tryptase and trypsin-3, that induce PAR₂-dependent hyperexcitability of nociceptors and colonic nociception in mice (Barbara, G. et al., Gastroenterology 2007, 132(1): 26-37; Cenac, N. et al., The Journal of Clinical Investigation 2007, 117(3):636-647; and Valdez-Morales, E. E. et al., Am J Gastroenterol 2013, 108(10): 1634-1643). PAR₂ agonists induce a remarkably long-lasting hyperexcitability of neurons by unknown mechanisms (Reed, D. E. et al., J Physiol 2003, 547(Pt 2): 531-542).

It is therefore evident that the development of potent and selective inhibitors of PAR₂ signaling is highly desirable and presents a substantial medical progress to advance treatment of inflammation, nociception, gastrointestinal motility, fibrosis, and cancer invasion.

### SUMMARY OF THE INVENTION

New compounds are provided that are suitable for the inhibition of PAR₂. The compounds of the present invention can be useful for the treatment and prevention of diseases and disorders mediated by this receptor. The PAR₂ inhibitors disclosed herein comprise a moiety that restricts their absorption, making them suitable for use in the treatment of diseases and disorders of the gastrointestinal tract as well as for targeted delivery of the compound.

In one aspect, the present invention provides a compound of Formula (I):

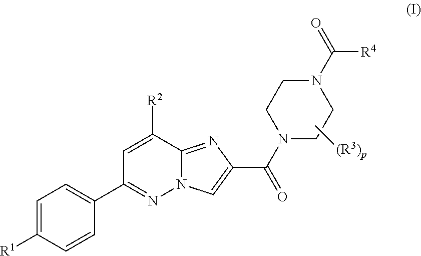

or pharmaceutically acceptable salt thereof, wherein:
R¹ is H, C₁-C₆ alkyl or halo;
R² is C₁-C₆ alkyl, C₃-C₆ cycloalkyl or C₁-C₆ aryl, each optionally substituted with 1 to 3 halogens;
R³ is oxo or C₁-C₆ alkyl;
p is an integer from 0 to 3;
R⁴ is —C₁-C₆ alkylS(O)₂OH, -1,2,3-triazol-1-acetic acid, —NHR⁷, -bicycle[2.2.2]octaneC(O)OR, —C₄-C₈ cycloalkyl-R⁵, a 4-6 membered heterocyclic or heteroaryl group substituted with —C₁-C₆ alkyl-R⁵, or —(CH₂)₂C(O)NHC₂-C₁₀ alkyl, wherein the C₂-C₁₀ alkyl is substituted with 2 to 10 —NH₂ or —OH; R⁵ is —C(O)NHR⁷ or —NHC(O)R⁷;
R⁶ is H or R⁷;
R⁷ is —R⁸, —C₁-C₂₀ alkyl, —C₁-C₂₀ alkylC(O)NH₂ or —C₁-C₂₀ alkylC(O)NR⁸, wherein the —C₁-C₂₀ alkyl, —C₁-C₂₀ alkylC(O)NH₂ and —C₁-C₂₀ alkylC(O)NR⁸ are optionally substituted with 2 to 10 —NH₂ or —OH, and wherein one or more of the carbon atoms in the alkyl group are optionally replaced with nitrogen or oxygen;
R⁸ is represented by the formula:

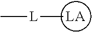

wherein
L is a linker moiety of 1 nm to 50 nm in length; and
LA is a lipid anchor that promotes insertion of the compound into a plasma membrane.

In another aspect, the present invention provides a method of inhibiting PAR₂ signaling comprising contacting the receptor with a compound of Formula (I) as herein defined, or a pharmaceutically acceptable salt thereof.

In a further aspect, the present invention provides a method of inhibiting PAR₂ signaling in a subject in need thereof, comprising administering to the subject an effective amount of a compound of Formula (I) as herein defined, or a pharmaceutically acceptable salt thereof.

In yet another aspect, the present invention provides a method for preventing or treating a disease or disorder mediated by PAR₂ signaling comprising administering to a subject in need thereof an effective amount of a compound of Formula (I) as herein defined, or a pharmaceutically acceptable salt thereof.

The present invention also provides a compound of Formula (I) as herein defined, or a pharmaceutically acceptable salt thereof, for the prophylaxis or treatment of a disease or disorder mediated by PAR₂ signaling.

In another aspect, the present invention provides use of a compound of Formula (I) as herein defined, or a pharmaceutically acceptable salt thereof, in the manufacture of a medicament for the prophylaxis or treatment of a disease or disorder mediated by PAR₂ signaling.

The present invention further provides a pharmaceutical composition comprising a compound of Formula (I) as herein defined or a pharmaceutically acceptable salt thereof, together with at least one pharmaceutically acceptable carrier or diluent.

These and other aspects of the present invention will become more apparent to the skilled addressee upon reading the following detailed description in connection with the accompanying Examples, Figures, and Claims.

### BRIEF DESCRIPTION OF THE DRAWINGS

FIG. 1: Protease-induced mechanical nociception. A. Localization of PAR₂ and Na_{V}1.8 immunoreactivities in DRG from WT or Par₂-Na_{V}1.8 mice. White arrowheads: neurons coexpressing PAR₂ and Na_{V}1.8 in WT mice. Yellow arrowheads: neurons expressing Na_{V}1.8 but not PAR₂ in Par₂-Na_{V}1.8 mice. B. Total number and number of trypsin (100 nm)-responsive DRG neurons (<25 μm) from WT and Par₂-Naᵥ1.8 mice. C-E. von Frey filaments withdrawal responses in WT and Par₂-Na_{V}1.8 mice after intraplantar injection of trypsin (C, Tryp), NE (D) or CS (E). F-K. von Frey filaments withdrawal responses in WT mice after intraplantar injection of Dy4 or Dy4 inact (F-H, dynamin inhibitor), PS2 or PS2 inact (J-K, clathrin inhibitor), or vehicle (Veh), followed 30 min later by intraplantar trypsin (F, I), NE (G, J) or CS (H, K). *P<0.05, **P<0.01, ***P<0.001, ****P<0.0001. Numbers in parentheses denote mouse numbers (N).

FIG. 1A: Protease-induced mechanical nociception. A. Localization of PAR₂ and Na_{V}1.8 immunoreactivities in DRG from WT or Par₂-Na_{V}1.8 mice. White arrowheads: neurons coexpressing PAR₂ and Na_{V}1.8 in WT mice. Yellow arrowheads: neurons expressing Na_{V}1.8 but not PAR₂ in Par₂-Na_{V}1.8 mice. B-D. von Frey withdrawal responses in WT and Par₂-Na_{V}1.8 mice after intraplantar injection of trypsin (B, Tryp), NE (C) or CS (D). E-F. von Frey withdrawal responses in WT mice after intraplantar injection of Dy4 or Dy4 inact (E-G, dynamin inhibitor), PS2 or PS2 inact (H-J, clathrin inhibitor), or vehicle (Veh), followed 30 min later by intraplantar trypsin (E, H), NE (F, I) or CS (G, J). *P<0.05, **P<0.01, ***P<0.001, ****P<0.0001. Numbers in parentheses denote mouse numbers (N).

FIG. 2: Protease-induced hyperexcitability of nociceptors. Rheobase of mouse DRG neurons preincubated with Dy4 (A, B, D, F, dynamin inhibitor), PS2 (C, E, and G, clathrin inhibitor) or vehicle control (Con). Neurons were challenged with trypsin (A-C), NE (D, E) or CS (F, G), washed, and rheobase was measured 0 or 30 min later. A. Representative traces. Rh, rheobase. B-G. Mean responses. *P<0.05, **P<0.01, ***P<0.001. Numbers in bars denote neuron numbers (N).

FIG. 2A: Protease-induced hyperexcitability of nociceptors. Rheobase of mouse DRG neurons preincubated with Dy4 (A, B, D, F, dynamin inhibitor), PS2 (C, E, G, clathrin inhibitor) or buffer control (Con). Neurons were challenged with trypsin (A-C), NE (D, E) or CS (F, G), washed, and rheobase was measured 0 or 30 min later. A. Representative traces. Rh, rheobase. B-G. Mean responses. *P<0.05, **P<0.01, ***P<0.001. Numbers in bars denote neuron numbers (N).

FIG. 3: Mechanisms of protease-induced hyperexcitability of nociceptors. Rheobase of mouse DRG neurons preincubated with I-343 (A-D, PAR₂ antagonist), PD98059 (E, MEK1 inhibitor), or GF109203X (F, GFX, PKC inhibitor). Neurons were challenged with trypsin (A, E, and F, Tryp), NE (B, D) or CS (C), washed, and rheobase was measured 0 or 30 min later. *P<0.05, **P<0.01, ***P<0.0001. Numbers in bars denote neuron numbers (N).

FIG. 4: PAR₂ endocytosis, βARR2 recruitment, and compartmentalized signalling in nociceptors. A-C. Endocytosis. A. Representative images (n=3 experiments) of effects of trypsin (Tryp) on the distribution of mPAR₂-GFP in mouse DRG neurons. Arrowheads (A, left) show PAR₂-GFP in endosomes. B, C. Cytosol/plasma membrane ratio of mPAR₂-GFP in mouse DRG neurons after 30 min incubation with trypsin, NE or CS (B), or after preincubated with Dy4 or Dy4 inact and then trypsin (C). D. PAR₂-RLuc8/βARR2-YFP BRET in mouse DRG neurons exposed to trypsin, NE or CS. AUC, area under curve (25 min) *P<0.05 to control. n, experimental replicates, triplicate observations. E-J. Compartmentalized signalling. Effects of trypsin on PKC activity at the plasma membrane (E and F) and in the cytosol (G), and on ERK activity in the cytosol (H and I), and nucleus (J) of rat DRG neurons: trypsin-induced activation of PKC at the plasma membrane (E, F) but not cytosol (G), and of ERK in the cytosol (H, I) and nucleus (J) of rat DRG neurons. Numbers in bars denote neuron numbers (N). *P<0.05, **P<0.01 compared to vehicle.

FIG. 5: PAR₂ endocytosis and compartmentalized ERK signaling in HEK293 cells. A-D. BRET assays of endocytosis. PAR₂-RLuc8/RIT-Venus BRET (A, C) and PAR₂-RLuc8/Rab5a-Venus BRET (B, D). E-K. FRET assays of cytosolic (E, G, H, J) and nuclear (F, G, I, K) ERK activity. AUC, area under curve. *P<0.05, **P<0.01, ***P<0.001, ****P<0.00001 compared with trypsin alone. n, experimental replicates, triplicate observations.

FIG. 6: IBS-D-induced hyperexcitability of nociceptors. A-D. Rheobase of mouse nociceptors 30 min after exposure to supernatant from biopsies of colonic mucosa from HC and IBS-D subjects. A. Representative traces of vehicle- or I-343-treated neurons. B-D. Mean responses of neurons preincubated with I-343 (B, PAR₂ antagonist), Dy4 (C, dynamin inhibitor), or PD98059 (D, MEKI inhibitor). E. PAR₂-RLuc8/Rab5a-Venus BRET in HEK293 cells measured after 60-min incubation with HC or IBS-D biopsy supernatant, or trypsin. *P<0.05, **P<0.01, ***P<0.001; ns, not significant. Numbers in bars denote neuron numbers (N).

FIG. 7: Targeting PAR₂ in endosomes of nociceptors. Representative images (of three experiments) of trafficking of Cy5 tripartite probes and mPAR₂-GFP to the soma (A) and neurites (B) of mouse DRG neurons. The scale bar (5 μm) in the bright-field image applies to all panels in the same row, except for Inset, which is a magnification of the dashed box in the merged panels. Arrows show proximity to vesicles containing mPAR₂-GFP and Cy5-Chol.

FIG. 8: Antagonism of endosomal PAR₂ and hyperexcitability of nociceptors. A, B. Trypsin-induced hyperexcitability of mouse DRG neurons. Neurons were preincubated with Compound 10 or vehicle (control, con) for 60 min, washed, and recovered for 170 or 140 min. Neurons were then exposed to trypsin (10 min). Rheobase was measured 0 or 30 min after trypsin and 180 min post-Compound 10. C. IBS-induced hyperexcitability of mouse DRG neurons. Neurons were preincubated with Compound 10 or vehicle (control, con) for 60 min, washed, and recovered for 60 min. Neurons were then exposed to HC or IBS-D supernatant for 30 min, washed and rheobase was measured 30 min later (T 30 min), 120 min post-Compound 10. *P<0.05, **P<0.01. Numbers in bars denote neuron numbers (N).

FIG. 9: Sensitization of colonic afferents and colonic nociception. A-H. Mechanosensory responses in healthy control mice to stimulation of the colonic mucosa with a 2 g VFF under basal conditions and after exposure of receptive fields to trypsin (A, B, and E-H, Tryp), NE (C and E), or CS (D and E). A. Representative results. B-D and F-H. Mean responses. E. Responses as percent basal. Numbers in bars denote afferent numbers. I and J. VMR to CRD in awake normal mice. Numbers in parentheses denote mouse number. *P<0.05, **P<0.01, ***P<0.001.

FIG. 10: Mechanisms of protease- and PAR₂-induced hyperexcitability of nociceptors. After activation by canonical mechanisms, PAR₂ signals at the plasma membrane to activate PKC, which mediates initial hyperexcitability (1). PAR₂ then undergoes clathrin-, dynamin-, and βARR-dependent endocytosis (2). PAR₂ continues to signal from endosomes by βARR-and Gaq-mediated mechanisms to activate ERK, which mediates persistent hyperexcitability. After activation by biased mechanisms, PAR₂ signals from the plasma membrane to activate adenylyl cyclase (AC) and PKA, which mediate the initial and persistent hyperexcitability (3). Kinases may regulate the activity of TRP channels and voltage-gated ion channels, to control nociceptor hyperexcitability (4).

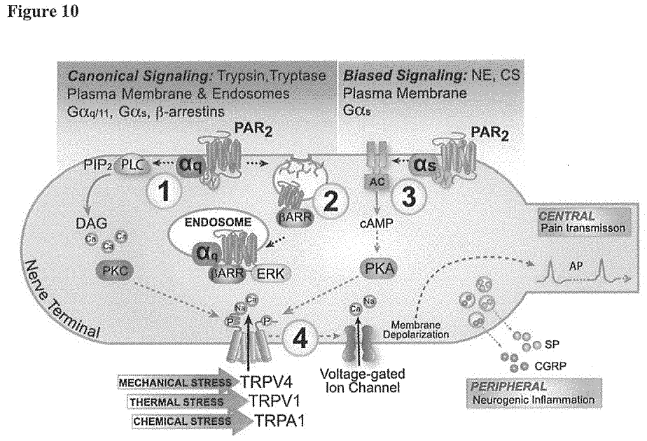

FIG. 11: Expression of functional PAR₂ in DRG neurons, and PAR₂-dependent inflammation. A, B. Representative traces of effects of trypsin (100 nM) on [Ca²⁺]ᵢ in DRG neurons from WT (A) and Par²-NaV1.8 (B) mice. Traces from 25 neurons are shown; traces from trypsin-responsive neurons are shown in red. In WT mice, 20/65 (31%) of neurons responded to trypsin. In Par₂-NaV1.8 mice, 3/51 (6%) of neurons responded to trypsin. Neurons were collected from 4 mice per group. C. Effect of intraplantar injection of trypsin on paw thickness in WT and Par₂-NaV1.8 mice. ***P<0.001, ****P<0.0001. Numbers in parentheses denote mouse numbers. D. Effect of intraplantar injection of trypsin on neutrophil infiltration into the paw at 4 h in WT and Par₂-NaV1.8 mice. Arrows show neutrophil influx in WT mice.

FIG. 12: Protease-induced mechanical nociception and edema. A, B. VFF withdrawal responses of the contralateral (right) paw after intraplantar injections into the ipsilateral (left) paw of Dy4 or Dy4 inact (A), PS2 or PS2 inact (B), or vehicle (Veh), followed by NE. C-H. Thickness of the ipsilateral paw. Dy4 or Dy4 inact (C, E, G), PS2 or PS2 inact (D, F, H), or vehicle (Veh) was administered by intraplantar injection into mouse paw. After 30 min, trypsin (Tryp) (C, D), NE (E, F) or CS (G, H) was injected. Paw thickness (edema) was measured. Numbers in parentheses denote mouse numbers.

FIG. 13: Endocytic inhibitors and baseline hyperexcitability of nociceptors. Rheobase of mouse DRG neurons preincubated with buffer control (Con), vehicle (Veh, 0.3% DMSO), Dy4 (A) or PS2 (B). Rheobase was measured at T 0 min or T 30 min after washing. Numbers in bars denote neuron numbers.

FIG. 14: Characterization of PAR₂ antagonist I-343. A. I-343 structure. B-D. Concentration-response analysis of the effects of I-343 on 2F- and trypsin-induced IP1 accumulation in HT-29 (B), HEK293 (C), and KNRK-hPAR2 (D) cells. E. Effects of I-343 on ATP-induced IP₁ accumulation in HEK cells. n, experimental replicates, triplicate observations.

FIG. 15: Trypsin- and thrombin-induced hyperexcitability of nociceptors. Rheobase of mouse DRG neurons preincubated with I-343 (A, PAR₂ antagonist) or SCH79797 (B, C, PAR₁ antagonist). Neurons were challenged with trypsin (A, C) or thrombin (B), washed, and rheobase was measured 0 min later. *P<0.05, **P<0.01. Numbers in bars denote neuron numbers.

FIG. 16: PAR₂ endocytosis in HEK293 cells. PAR₂-RLuc8/RIT-Venus BRET (A, B, E, G) and PAR₂-RLuc8/Rab5a-Venus BRET (C, D, F, H) in HEK293 cells. n, experimental replicate, triplicate observations.

FIG. 17: PAR₂ compartmentalized ERK signaling in HEK293 cells. FRET assays of cytosolic (A-C, G, I, K) and nuclear (D-F, H, J, L) ERK activity in HEK293 cells. B, E. Sensor localization. n, experimental replicates, triplicate observations.

FIG. 18: Trafficking of PAR₂, βARR1 and Gα_{q} to early endosomes in HEK293 cells. A, B. βARR1-RLuc8/Rab5a-Venus BRET (A) and Gα_{q}-RLuc8/Rab5a-Venus BRET (B) in HEK293 cells. *P<0.05, ***P<0.001 compared to vehicle. n, experimental replicates, triplicate observations. C. Localization of EEA1, Gα_{q}, and PAR₂ in endosomes after treatment with vehicle or trypsin for 30 min. Arrow heads show colocalization of EEA1, Gα_{q}, and PAR₂ in endosomes of trypsin-treated cells.

FIG. 19: PAR₂ compartmentalized PKC and cAMP signaling in HEK293 cells. FRET assays of cytosolic PKC (A, E, and G), plasma membrane PKC (B, E, and G), cytosolic cAMP (C, F, and H) and plasma membrane cAMP (D, F, and H) in HEK293 cells. I-L. Sensor localization. AUC, area under curve. *P<0.05, **P<0.01 compared to control. n, experimental replicates, triplicate observations.

FIG. 20: Tripartite PAR₂ antagonist. A. Principal of targeting PAR₂ in endosomes using a tripartite probe. B. Structure of Compound 10 tripartite PAR₂ antagonist. C. Concentration-response analysis of the effects of I-343 and Compound 10 on 2F-induced IP₁ accumulation in HT-29 cells.

FIG. 21: Sensitization of colonic afferents and colonic compliance. A-D. Mechanosensory responses of mice measured 28 d after exposure to TNBS. The colonic mucosa was stimulated with a 2 g von Frey filaments under basal conditions and after exposure of receptive fields to trypsin (A, D, Tryp), NE (B, D) or CS (C, D). D. Responses as % basal. Numbers in bars denote afferent numbers. E, F. Colonic compliance in awake healthy control mice. Pressure/volume relationships were unchanged by a protease cocktail (E) or by I-343 (F), which indicates that compliance of the colon is unchanged. Numbers in parentheses denote mouse numbers. *P<0.05, **P<0.01.

### DETAILED DESCRIPTION OF THE INVENTION

A new series of compounds is described herein that differ most significantly from known PAR₂ modulators in that they comprise a moiety specifically to control delivery of the inhibitor. The moiety is designed either to control the absorption of the compound across the intestinal lumen and subsequent systemic exposure of the compounds, or to allow for the targeted delivery of the compound.

Non-absorbed or non-systemic pharmaceutical agents acting within the intestinal lumen have found wide use in the treatment of systemic metabolic disorders as well as in the treatment of diseases and disorders of the gastrointestinal tract (Charmot, D., Current Pharmaceutical Designs 2012, 18, 1434-1445). Non-absorbed agents are also advantageous in that they minimize off-target systemic effects and thereby offer favorable toxicity profiles with reduced side effects. It is envisaged that the compounds of the invention may be particularly useful in the treatment of diseases and disorders of the GI system associated with undesired PAR₂ activity including, but not limited to, gastrointestinal motility, diet-induced obesity, inflammatory bowel diseases, irritable bowel syndrome and pain associated with irritable bowel syndrome.

The absorption of systemic agents generally proceeds by passive or active transport within enterocytes lining the intestinal lumen or by passive paracellular transport through cellular tight junctions. Without wishing to be limited by theory, and with reference to compounds of Formula (I):

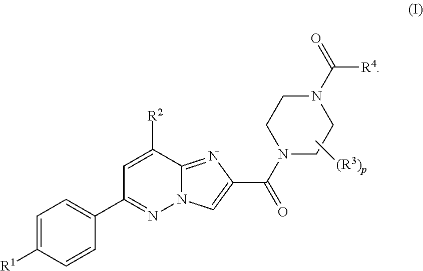

it has now been found that the addition of certain groups at variable R⁴ restrict luminal absorption of the resultant compound while maintaining inhibitory activity against PAR₂. These groups include, but are not limited to, —C₁-C₆ alkylS(O)₂OH, -1,2,3-triazol-1-acetic acid, —NHR⁷, -bicycle[2.2.2]octaneC(O)OR, —C₄-C₈ cycloalkyl-R⁵, a 4-6 membered heterocyclic or heteroaryl group substituted with —C₁-C₆ alkyl-R⁵, or —(CH₂)₂C(O)NHC₂-C₁₀ alkyl, wherein the C₂-C₁₀ alkyl is substituted with 2 to 10 —NH₂ or —OH.

In another embodiment, certain groups at R⁴ act to enable the target delivery of the compounds of the invention to PAR₂ receptors that have been endocytosed into early endosomes.

Endosomal signaling of PAR₂ has been evaluated for its role in pain suffered by patients with irritable bowel syndrome (IBS). Trypsin, elastase, and cathepsin S, which are activated in the colonic mucosa of patients with IBS and in experimental animals with colitis, caused persistent PAR₂-dependent hyperexcitability of nociceptors, sensitization of colonic afferent neurons to mechanical stimuli, and somatic mechanical allodynia. Inhibitors of clathrin- and dynamin-dependent endocytosis and of mitogen-activated protein kinase kinase prevented trypsin-induced hyperexcitability, sensitization, and allodynia. However, they did not affect elastase- or cathepsin S-induced hyperexcitability, sensitization or allodynia. Trypsin stimulated endocytosis of PAR₂, which signaled from endosomes to activate extracellular signal regulated kinase. Elastase and cathepsin S did not stimulate endocytosis of PAR₂, which signaled from the plasma membrane to activate adenylyl cyclase. Biopsies of colonic mucosa from IBS patients released proteases that induced persistent PAR₂-dependent hyperexcitability of nociceptors, and PAR₂ association with β-arrestins, which mediate endocytosis. Compounds of the invention incorporating a lipid anchor such as cholestanol promote delivery and retention of the compound in endosomes containing PAR₂. A compound of the invention prevented persistent trypsin- and IBS protease-induced hyperexcitability of nociceptors. These results reveal that PAR₂ signaling from endosomes underlies the persistent hyperexcitability of nociceptors that mediates chronic pain of IBS. Inhibitors of endosomal PAR₂ signaling may therefore provide a novel therapy for IBS pain.

The term “endosomal PAR₂ signaling” as herein used refers to the signal transduced by activated PAR₂ that has been endocytosed into an endosome, preferably an early endosome.

The term “inhibiting endosomal PAR₂ signaling” as herein used refers to antagonists or inhibitors of PAR₂ that act (or continue to act) at the receptor after it has been endocytosed into an endosome.

In order to target endosomal PAR₂ signaling, the compounds of the invention are prepared as “tripartite compounds” comprising the moiety:

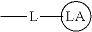

wherein
L is a linker moiety of 1 nm to 50 nm in length; and
LA is a lipid anchor that promotes insertion of the compound into a plasma membrane.

The term “tripartite compound” as herein used refers to compounds of Formula (I) as herein described, or pharmaceutically acceptable salts thereof, comprising an inhibitor of PAR₂ covalently bound to a linker group, the linker group being covalently bound to a lipid anchor capable of anchoring the inhibitor of PAR₂ to the lipid bilayer of a cell membrane and ultimately, to the membrane of an early endosome.

The term “lipid anchor” (LA) as herein used denotes moieties that are capable of partitioning into lipid membranes and thereby anchoring the compound of Formula (I) into the lipid membrane. The partition into the lipid membrane may occur directly from the extracellular or vesicular luminal space or may occur laterally from the lipid bilayer.

In one preferred embodiment, the lipid anchor may be characterized by its ability to partition into lipid membranes, whereby said lipid membranes are characterized by insolubility in non-ionic detergents at 4° C.

Examples of suitable lipid anchors include, but are not limited to cholesterol, cholestanol, sphingolipid, GPI-anchor or saturated fatty acid derivatives. Many such lipid anchors have been described in the art, for example, in WO2005/097199, the entirety of which is incorporated herein by reference.

In one embodiment, the lipid anchor is a moiety selected from formulae (IIa), (IIIa), (IIIa-2), and (IVa):

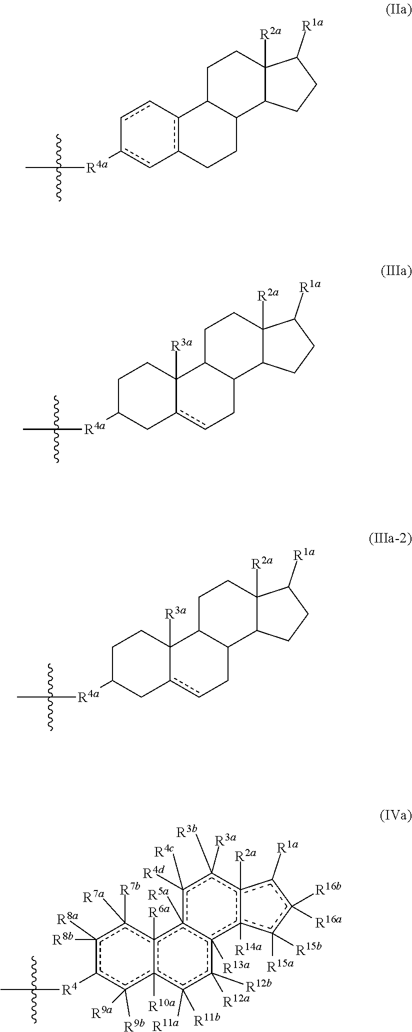

wherein
R^{1a} is an optionally substituted C₁₋₁₂ alkyl, alkenyl, alkynyl or alkoxy group;
R^{2a} and R^{3a}, R^{3b}, R^{4b}, R^{4c}, R^{5a}, R^{6a}, R^{7a}, R^{7b} R^{8a}, R^{8b}, R^{9a}, R^{9b}, R^{10a}, R^{11a}, R^{11b}, R^{12a}, R^{12b} R^{13a}, R^{14a}, R^{15a}, R^{15b}, R^{16a} and R^{16b} are independently H, C₁₋₃ alkyl, hydroxyl, C₁₋₃ alkoxy or amino; or
optionally, R^{3a}, R^{3b} and/or R^{4b}, R^{4c}, and/or R^{7a}, R^{7b} and/or R^{8a}, R^{8b} and/or R^{11a}, R^{11b} and/or R^{12a}, R^{12b} and/or R^{15a}, R^{15b} and R^{16a}, R^{16b} are taken together to give ═O (double bond to oxygen);

### R^{4a} is C, O, NH or S;

[symbol] represents a single or double bond; or
a pharmaceutically acceptable salt thereof.

In other embodiments, the lipid anchor is a moiety selected from formulae (Va), (VIa), (VIIa) or (VIIIa):

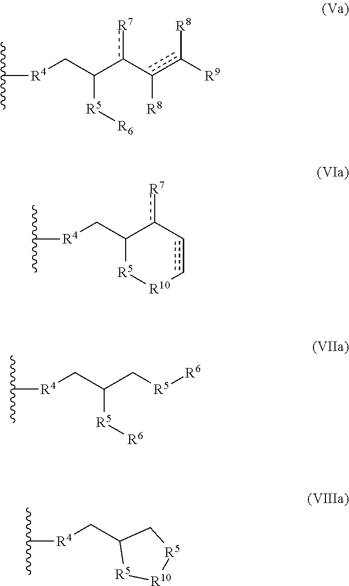

wherein
R⁴ is as described above; [symbol]
represents a single or double bond; [symbol]
represents a single, double or triple bond;
each occurrence of R⁵ is independently —NH—, —O—, —S—, —OC(O)—, —NHC(O)—, —NHCONH—, —NHC(O)O— or —NHS(O₂)—;
each occurrence of R⁶ is independently a C₁₄₋₃₀ alkyl group optionally substituted by fluorine, preferably 1 to 4 fluorine atoms;
each occurrence of R⁷ is independently NH₂, NHCH₃, OH, H, halogen or O, provided that when R⁷ is NH₂, NHCH₃, OH, H or halogen then [symbol] is a single bond and when R is O then [symbol] is a double bond;
each occurrence of R⁸ is independently H, OH or is absent when [symbol] represents a triple bond;
R⁹ is a C₁₀₋₃₀ alkyl group optionally substituted by fluorine, preferably 1 to 4 fluorine atoms; and
each occurrence of R¹⁰ is independently a C₂₄₋₄₀ alkylene group, a C₂₄₋₄₀ alkenylene group or a C₂₄₋₄₀ alkynylene group optionally substituted by fluorine, preferably 1 to 4 fluorine atoms; or
a pharmaceutically acceptable salt thereof.

In further embodiments, the lipid anchor is a moiety selected from formulae (IXa) or (Xa):

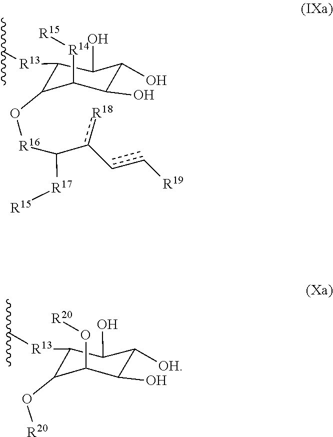

wherein [symbol]
represents a single or double bond; [symbol]
represents a single, double or triple bond;
each occurrence of R¹³ is independently —O— or —CO(CH₂)ₐ(CO)_{b}O—, wherein a is an integer from 1 to 3 and b is an integer from 0 to 1;

### R¹⁴ is —O— or —OC(O)—;

each occurrence of R¹⁵ is independently selected from a C₁₆₋₃₀ alkyl group optionally substituted with fluorine, preferably 1 to 4 fluorine atoms;
R¹⁶ is —PO₃⁻CH₂—, —SO₃CH₂—, —CH₂—, —CO₂CH₂— or a direct bond;
R¹⁷ is —NH—, —O—, —S—, —OC(O)—, —NHC(O)—, —NHCONH—, —NHC(O)O— or —NHS(O₂)—;
R¹⁸ NH₂, NHCH₃, OH, H, halogen or O;
R¹⁹ is a C₁₆₋₃₀ alkyl group optionally substituted with fluorine, preferably 1 to 4 fluorine atoms; and
each R²⁰ is a C(O)C₁₃₋₂₅alkyl group optionally substituted with a group of the following formulae:

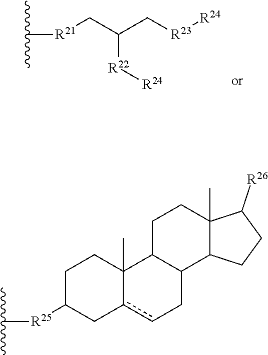

wherein [symbol]
is a single or double bond;
R²¹ is —PO₃⁻—CH₂—, —SO₃CH₂, —CH₂—, —CO₂CH₂— or a direct bond;
R²² is —NH—, —O—, —S—, —OC(O)—, —NHC(O)—, —NHCONH—, —NHC(O)O— or —NHS(O₂)—;

### R²³ is —O— or —OC(O)—;

each occurrence of R²⁴ is independently selected from a C₁₆₋₃₀ alkyl group optionally substituted with fluorine, preferably 1 to 4 fluorine atoms;
R²⁵ is —CO(CH₂)ₐ(CO)_{b}O— or —CO(CH₂)ₐ(CO)_{b}NH—, wherein a is an integer from 1 to 3 and b is an integer from 0 to 1; and
R²⁶ is a C₄₋₂₀ alkyl group optionally substituted with fluorine, preferably 1 to 4 fluorine atoms; or
a pharmaceutically acceptable salt thereof.

In further embodiments the lipid anchor is a moiety selected from formulae (XIa), (XIIa), (XIIIa) or (XIVa):

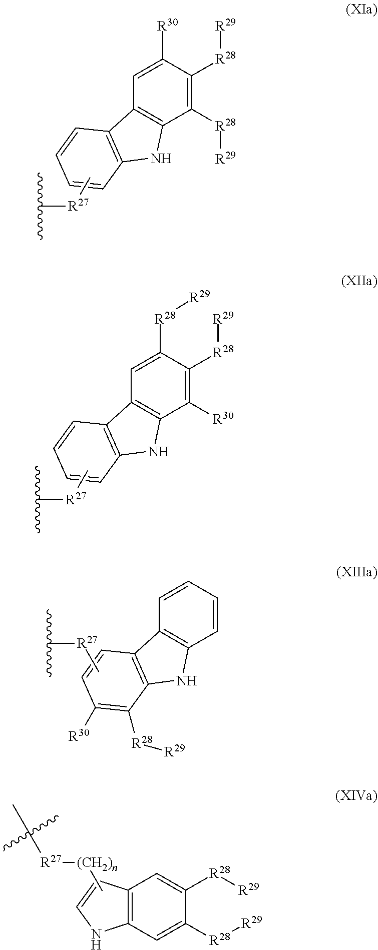

wherein
each occurrence of R²⁷ is independently selected from —NH—, —O—, —NH(CH₂)_{c}OPO₃⁻—, —NH(CH₂)_{c}SO₂NH—, —NHCONH—, —NHC(O)O—, CO(CH₂)_{b}(CO)ₐNH—, —CO(CH₂)_{b}(CO)ₐO—, —CO(CH₂)_{b}S—, —CO(CH₂)_{b}OPO₃⁻—, —CO(CH₂)_{b}SO₂NH—, —CO(CH₂)_{b}NHCONH—, —CO(CH₂)_{b}OCONH—, —CO(CH₂)_{b}OSO₃⁻—, or —CO(CH₂)_{b}NHC(O)O—, wherein a is an integer from 0 to 1, b is an integer from 1 to 3 and c is an integer from 2 to 3;
each occurrence of R²⁸ is independently —CH₂— or —O—;
each occurrence of R²⁹ is independently selected from H or a C₁₆₋₃₀ alkyl group optionally substituted by fluorine, preferably 1 to 4 fluorine atoms;
each occurrence of R³¹ is independently selected from H, or a C₁₋₁₅ alkyl group, optionally substituted by fluorine, preferably 1 to 4 fluorine atoms, or a C₁₋₁₅ alkoxy group optionally substituted by fluorine, preferably 1 to 4 fluorine atoms; and
n is an integer from 1 to 2; or
a pharmaceutically acceptable salt thereof.

In still further embodiments, the lipid anchor moiety is a C₁₋₂₀ alkyl (e.g., C₁₆ alkyl).

The term “linker” as herein used relates to the part of the compound that links the PAR₂ inhibitor to the lipid anchor. It will be understood that the linker should be selected such that it does not compete with the PAR₂ inhibitor at the ligand binding site. Nor should the linker partition into the lipid membrane.

The linker group should be of a length of between 1 nm to 50 nm in order to allow the inhibitor of PAR₂ to interact with the receptor when the lipid anchor is anchored in the endosome membrane.

In one embodiment, the linker group will comprise one or more polyethelene glycol units. In another embodiment it is envisaged that the linker, or subunits of the linker, may be amino acid residues, derivatised or functionalised amino acid residues, polyethers, ureas, carbamates, sulphonamides or other subunits that provide adequate distance between the PAR₂ inhibitor and the lipid anchor without interfering in the function of either group. In one embodiment, the linker is represented by a moiety of the formula (XVa):

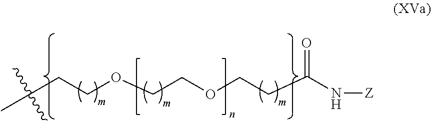

wherein
Z is the attachment group between the linker and the lipid anchor and is —C₁-C₁₀ alkyl-, —C₂-C₁₀ alkenyl-, —C₂-C₁₀ alkynyl-, —C₁-C₁₀ alkylC(O)—, —C₂-C₁₀ alkenylC(O)— or —C₂-C₁₀ alkynylC(O)—; or
Z, together with the adjacent amine, is an optionally C-terminal modified (e.g., C-terminal is amidated or C-terminal is an acyl hydrazine

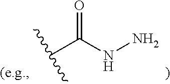 [SMILES: *C(=O)NN]

amino acid selected from aspartic acid, glutamic acid, asparagine, glutamine, histidine, cysteine, lysine, arginine, serine or threonine; wherein the amino acid is attached to the lipid anchor via its side-chain functional group;
m is 1 or 2;
n is from 1 to 20; and
p is from 1 to 8; or
a pharmaceutically acceptable salt thereof.

In one embodiment, the linker is represented by a moiety of the formula (XVa):

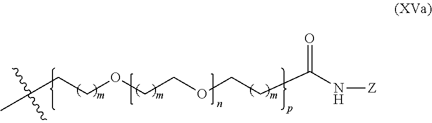

wherein
Z is the attachment group between the linker and the lipid anchor and is —C₁-C₁₀ alkyl-, —C₂-C₁₀ alkenyl-, —C₂-C₁₀ alkynyl-, —C₁-C₁₀ alkylC(O)—, —C₂-C₁₀ alkenylC(O)— or —C₂-C₁₀ alkynylC(O)—; or
Z, together with the adjacent amine, is an optionally C-terminal amidated amino acid selected from aspartic acid, glutamic acid, asparagine, glutamine, histidine, cysteine, lysine, arginine, serine or threonine; wherein the amino acid is attached to the lipid anchor via its side-chain functional group;
m is 1 or 2;
n is from 1 to 20; and
p is from 1 to 8; or
a pharmaceutically acceptable salt thereof.

In another embodiment, the linker is represented by a moiety of the formula (XVIa):

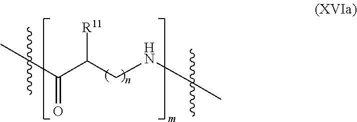

wherein
each occurrence of R¹¹ is independently any side chain of a naturally occurring, derivatised or functionalised amino acid residue;
m is an integer from 3 to 80; and
n is an integer from 0 to 1; or
a pharmaceutically acceptable salt thereof.

In other embodiments, the linker is represented by a moiety of the formula (XVIIa):

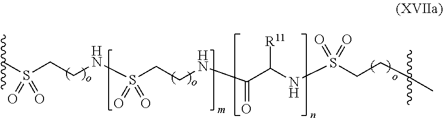

wherein
m is an integer from 0 to 40;
n is an integer from 0 to 1;
each occurrence of o is independently an integer from 1 to 5;
each occurrence of R¹¹ is independently any side chain of a naturally occurring, derivatised or functionalised amino acid residue; and
wherein the SO₂ terminus is bound to the lipid anchor.

In a further embodiment, the linker is represented by a moiety of the formula (XVIIIa):

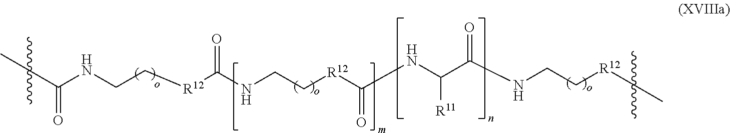

wherein
m is an integer from 0 to 40;
n is an integer from 0 to 1;
each occurrence of o is independently an integer from 1 to 5;
each R¹² is independently NH or O;
each occurrence of R¹¹ is independently any side chain of a naturally occurring, derivatised or functionalised amino acid residue; and
wherein the C(O)-terminus is bound to the lipid anchor and the R¹²-terminus is bound to the inhibitor of endosomal PAR2 signaling.

A number of suitable linker moieties have been described WO2005/097199, the entirety of which is incorporated herein by reference.

In this specification a number of terms are used which are well known to a skilled addressee. Nevertheless, for the purposes of clarity a number of terms will be defined.

As used herein, the term “alkyl”, used either alone or in compound words, denotes straight chain or branched alkyl. Prefixes such as “C₁₋₁₂” are used to denote the number of carbon atoms within the alkyl group (from 1 to 12 in this case). Examples of straight chain and branched alkyl include methyl, ethyl, n-propyl, isopropyl, n-butyl, sec-butyl, t-butyl, n-pentyl, hexyl, heptyl, 5-methylheptyl, 5-methylhexyl, octyl, nonyl, decyl, undecyl, dodecyl and docosyl (C₂₂).

The term “alkenyl”, used either alone or in compound words, denotes straight chain or branched hydrocarbon residues containing at least one carbon to carbon double bond including ethylenically mono-, di- or polyunsaturated alkyl groups as previously defined. Prefixes such as “C₂₋₁₂” are used to denote the number of carbon atoms within the alkenyl group (from 2 to 12 in this case). Examples of alkenyl include vinyl, allyl, 1-methylvinyl, butenyl, iso-butenyl, 3-methyl-2-butenyl, 1-pentenyl, 1-hexenyl, 3-hexenyl, 1-heptenyl, 3-heptenyl, 1-octenyl, 1-nonenyl, 2-nonenyl, 3-nonenyl, 1-decenyl, 3-decenyl, 1,3-butadienyl, 1,4-pentadienyl, 1,3-hexadienyl, 1,4-hexadienyl and 5-docosenyl (C₂₂).

The term “alkynyl”, used either alone or in compound words, denotes straight chain or branched hydrocarbon residues containing at least one carbon to carbon triple bond. Prefixes such as “C₂-C₁₀” are used to denote the number of carbon atoms within the alkenyl group (from 2 to 10 in this case).

As used herein, the term “aryl” denotes an optionally substituted monocyclic, or fused polycyclic, aromatic carbocyclic (ring structure having ring atoms that are all carbon) preferably having from 5 to 12 atoms per ring. Examples of aryl groups include monocyclic groups such as phenyl, fused polycyclic groups such as naphthyl, and the like.

The term “heteroaryl”, as used herein, represents a monocyclic or bicyclic ring, typically of up to 7 atoms in each ring, wherein at least one ring is aromatic and contains from 1 to 4 heteroatoms selected from the group consisting of O, N and S. Heteroaryl groups within the scope of this definition include but are not limited to: benzimidazole, acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indoiyl, pyrazinyl, pyridazinyl, pyridinyl, pyrimidinyl, pyrrolyl, 1H-1,2,3-triazole, 2H-1,2,3-triazole, 1H-1,2,4-triazole and tetrahydroquinoline.

As used herein, the term “heterocycle” or “heterocyclyl”, used either alone or in compound words, denotes saturated or partially unsaturated monocyclic, bicyclic or fused polycyclic ring systems containing at least one heteroatom selected from the group consisting of O, N and S. Prefixes such as “C₄-C₈” are used to denote the number of carbon atoms within the cyclic portion of the group (from 4 to 8 in this case). “Heterocycle” includes dihydro and tetrathydro analogs of the above mentioned heteroaryl groups. Examples of suitable heterocyclic substituents include, but are not limited to, pyrroline, pyrrolidine, piperidine, piperazine, pyrazoline, pyrazolidine, imidazolidine, tetrahydrofuran, pyran, dihydropyran, tetrahydropyran, dioxane, oxalzoline, morpholine, thiomorpholine, tetrahydrothiophene, oxathiane, dithiane, 4H-1,2,3-triazole and dithiazine, each of which may be further substituted with 1 to 3 substituents.

The term “halo” used herein refers to fluoro, chloro, bromo or iodo.

The term “oxo” denotes an oxygen atom divalently bound to the adjacent carbon atom. It will be understood that when an “R” variable is oxo, the hydrogen atom implied for the adjacent carbon atom in the cyclic structure will be absent because of the divalent nature of oxo.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

In some preferred embodiments of the invention, and with reference to the general Formula (I), one or more of the following preferments apply:

a) R¹ is H, C₁-C₆ alkyl or halo.
b) R¹ is halo.
c) R¹ is fluoro
d) R² is C₁-C₆ alkyl, C₃-C₆ cycloalkyl or C₁-C₆ aryl, each optionally substituted with 1 to 3 halogens.
e) R² is C₄ alkyl.
f) R² is t-butyl.
g) R³ is oxo or C₁-C₆ alkyl and p is an integer from 0 to 3.
h) R³ is C₁-C₆ alkyl and p is 2.
i) R³ is methyl and p is 2.
j) R⁴ is —C₁-C₆ alkylS(O)₂OH, -1,2,3-triazol-1-acetic acid, —NHR⁷, -bicycle[2.2.2]octaneC(O)OR, —C₄-C₈ cycloalkyl-R⁵, a 4-6 membered heterocyclic or heteroaryl group substituted with —C₁-C₆ alkyl-R⁵, or —CH₂)₂C(O)NHC₂-C₁₀ alkyl, wherein the C₂-C₁₀ alkyl is substituted with 2 to 10 —NH₂ or —OH.
k) R⁵ is —C(O)NHR⁷ or —NHC(O)R⁷;
i) R⁶ is H or R⁷
j) R⁷ is —R⁸, —C₁-C₂₀ alkyl, —C₁-C₂₀ alkylC(O)NH₂ or —C₁-C₂₀ alkylC(O)NR⁸, wherein the —C₁-C₂₀ alkyl, —C₁-C₂₀ alkylC(O)NH₂ and —C₁-C₂₀ alkylC(O)NR⁸ are optionally substituted with 2 to 10 —NH₂ or —OH, and wherein one or more of the carbon atoms in the alkyl group are optionally replaced with nitrogen or oxygen.
k) R⁷ is —C₁-C₂₀ alkyl, —C₁-C₂₀ alkylC(O)NH₂ or —C₁-C₂₀ alkylC(O)NR⁸, wherein the —C₁-C₂₀ alkyl, —C₁-C₂₀ alkylC(O)NH₂ and —C₁-C₂₀ alkylC(O)NR⁸ are optionally substituted with 2 to 10 —NH₂ or —OH, and wherein one or more of the carbon atoms in the alkyl group are optionally replaced with nitrogen or oxygen.
l) R⁷ is —R⁸.
m) R⁸ is represented by the formula:

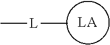

wherein
L is a linker moiety of 1 nm to 50 nm in length; and
LA is a lipid anchor that promotes insertion of the compound into a plasma membrane
n) LA is a lipid anchor selected from cholesterol, cholestanol, sphingolipid, a GPI-anchor or a saturated fatty acid derivative.
o) LA is a lipid anchor selected from moieties of formulae (IIa), (IIIa), (IVa), (Va), (VIa), (VIIa), (VIIIa), (IXa), (Xa), (XIa), (XIIa), (XIIIa), and (XIVa).
p) LA is a lipid anchor selected from moieties of formulae (IIa) or (IIIa).
q) L is a linker moiety comprising one or more subunits, the subunits comprising polyethelene glycol units, amino acid residues, derivatised or functionalised amino acid residues, polyethers, ureas, carbamates and/or sulphonamides.
r) L is a linker moiety represented by formulae (XVa), (XVIa), (XVIIa) or (XVIIIa).
s) L is a linker moiety represented by formula (XVa).

In a preferred embodiment, compounds of Formula (I), or pharmaceutically acceptable salts thereof, are selected from:

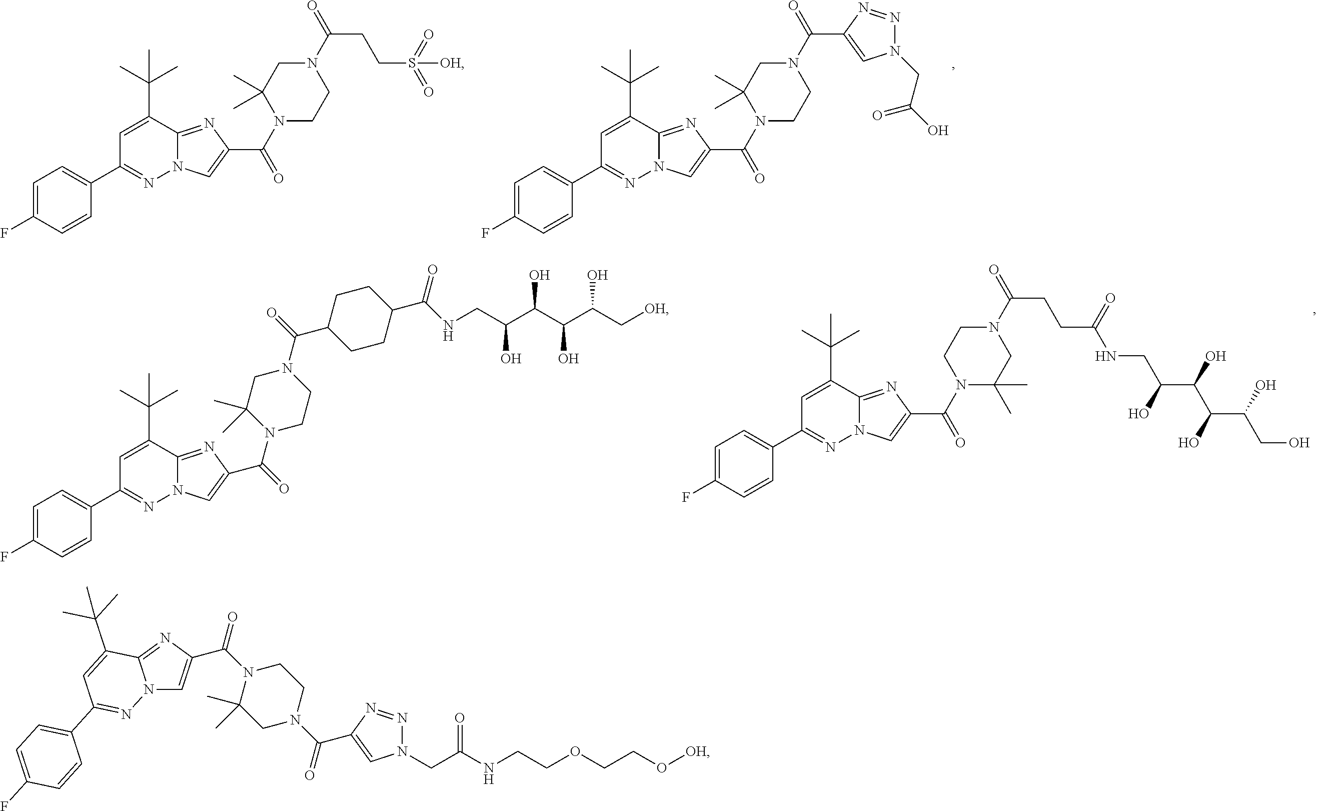

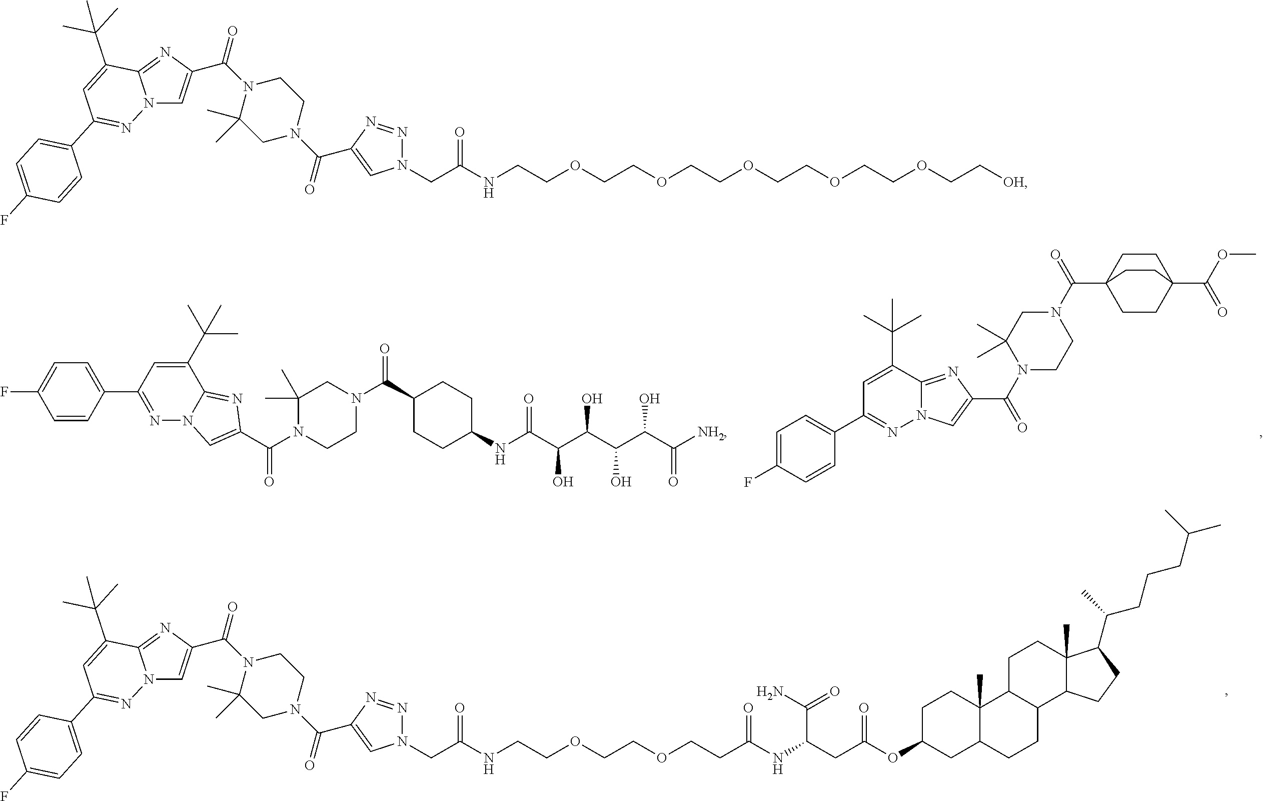

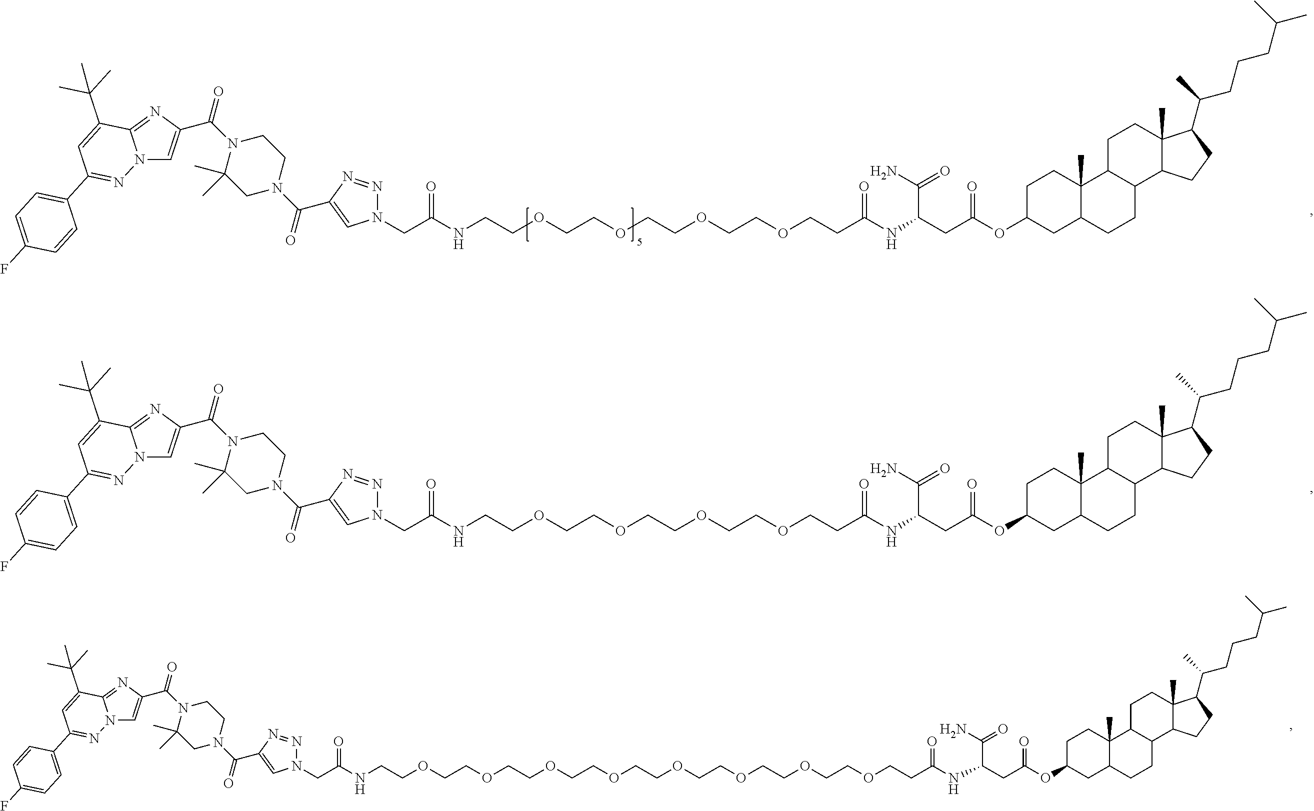

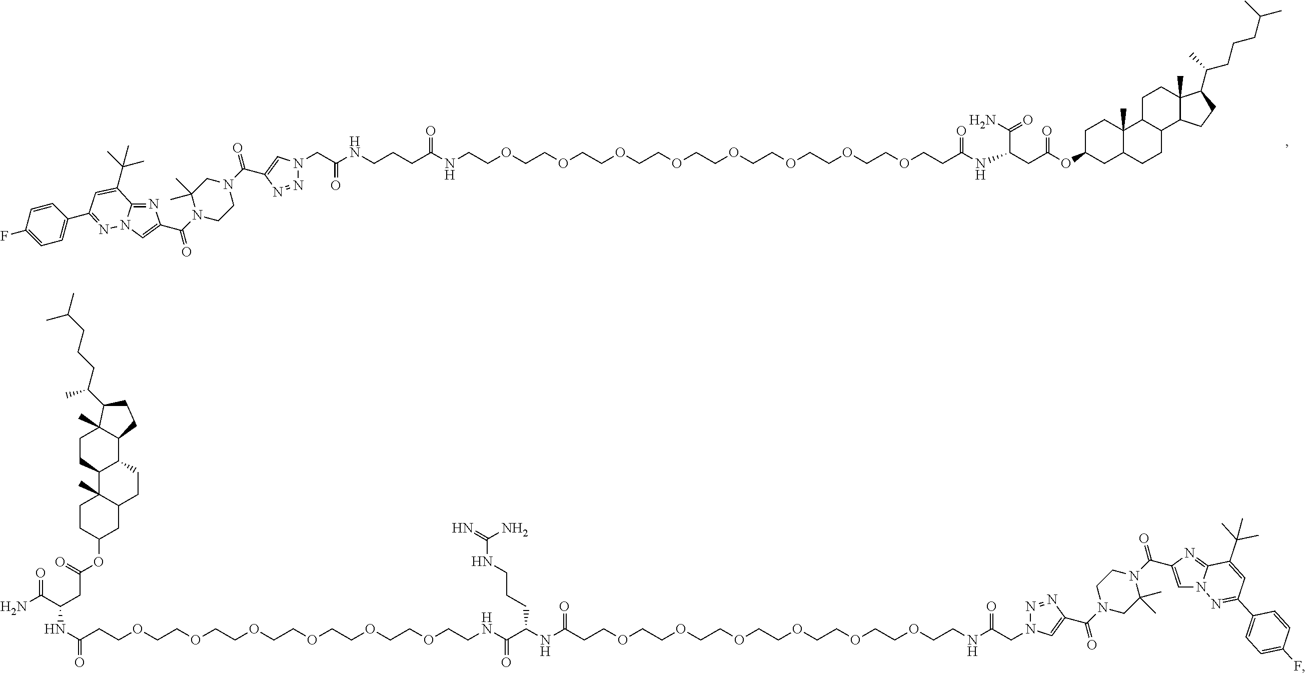

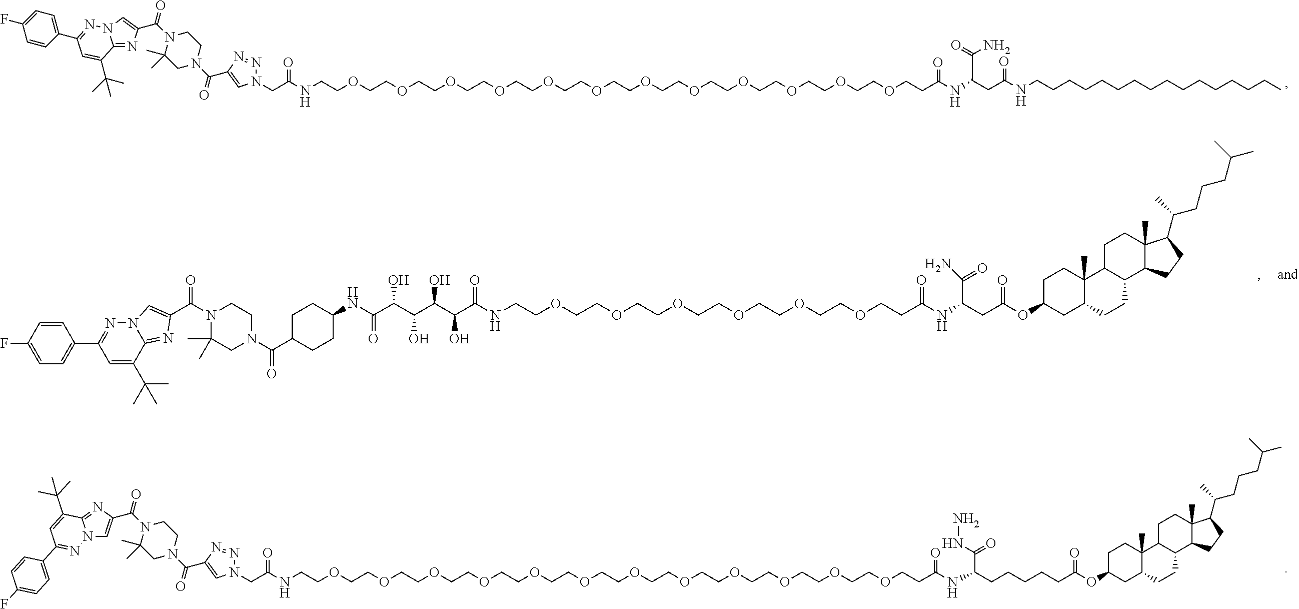

It will be understood that the compounds of the present invention may exist in one or more stereoisomeric forms (e.g., diastereomers). The present invention includes within its scope all of these stereoisomeric forms either isolated (in, for example, enantiomeric isolation), or in combination (including racemic mixtures and diastereomic mixtures).

The invention thus also relates to compounds in substantially pure stereoisomeric form with respect to asymmetric chiral centres, e.g., greater than about 90% de, such as about 95% to 97% de, or greater than 99% de, as well as mixtures, including racemic mixtures, thereof. Such diastereomers may be prepared by asymmetric synthesis, for example, using chiral intermediates, or mixtures may be resolved by conventional methods, e.g., chromatography, or use of a resolving agent.

The present invention contemplates the use of amino acids in both L and D forms, including the use of amino acids independently selected from L and D forms, for example, where the peptide comprises two serine residues, each serine residue may have the same, or opposite, absolute stereochemistry. Unless stated otherwise, the amino acid is taken to be in the L-configuration.

Where the compound comprises one or more functional groups that may be protonated or deprotonated (for example at physiological pH) the compound may be prepared and/or isolated as a pharmaceutically acceptable salt. It will be appreciated that the compound may be zwitterionic at a given pH. As used herein the expression “pharmaceutically acceptable salt” refers to the salt of a given compound, wherein the salt is suitable for administration as a pharmaceutical. Such salts may be formed, for example, by the reaction of an acid or a base with an amine or a carboxylic acid group, respectively.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Pharmaceutically acceptable base addition salts may be prepared from inorganic and organic bases. Corresponding counter ions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine.

Acid/base addition salts tend to be more soluble in aqueous solvents than the corresponding free acid/base forms.

The compounds of the invention may be in crystalline form or as solvates (e.g., hydrates) and it is intended that both forms are within the scope of the present invention. The term “solvate” is a complex of variable stoichiometry formed by a solute (in this invention, a peptide of the invention) and a solvent. Such solvents should not interfere with the biological activity of the solute. Solvents may be, by way of example, water, ethanol, or acetic acid. Methods of solvation are generally known within the art.

The compounds of the invention may be in the form of a pro-drug. The term “pro-drug” is used in its broadest sense and encompasses those derivatives that are converted in vivo to the compounds of the invention. Such derivatives would readily occur to those skilled in the art and include, for example, compounds where a free hydroxy group is converted into an ester derivative or a ring nitrogen atom is converted to an N-oxide. Examples of ester derivatives include alkyl esters (for example acetates, lactates, and glutamines), phosphate esters, and those formed from amino acids (for example valine). Any compound that is a prodrug of a compound of the invention is within the scope and spirit of the invention. Conventional procedures for the preparation of suitable prodrugs according to the invention are described in text books, such as “Design of Prodrugs” Ed. H. Bundgaard, Elsevier, 1985, the entire contents of which is incorporated herein by reference.

In one embodiment of the present invention, there is provided a method of inhibiting PAR₂ signaling comprising contacting PAR₂ with a compound of Formula (I) as herein defined or a pharmaceutically acceptable salt thereof. The exposing of the cell to the compound or pharmaceutically acceptable salt thereof may occur in vitro, ex vivo, or in vivo.

Where the exposing of a cell to the compound occurs in vitro or ex vivo, for example, the method of the present invention may be used as a tool for biological studies or as a diagnostic tool to determine the efficacy of certain compounds (alone or in combination) for modulating PAR₂ activity in a subject. As an example, a cell that expresses PAR₂ may be removed from a subject and exposed to one or more compounds of the present invention, or salts thereof. The ability of the compound (or compounds) to modulate the activity of PAR₂ can be assessed by measuring any one of a number of down stream markers via a method known to one skilled in the art. Thus, one may be able to ascertain whether a certain compound is more efficacious than another and tailor a specific treatment regime to that subject.

In a preferred embodiment there is provided a method for preventing or treating a disease or disorder mediated by PAR₂ signaling comprising administering to a subject in need thereof an effective amount of a compound of Formula (I) as herein defined, or a pharmaceutically acceptable salt thereof.

In a particular preferred embodiment, the present invention provides a method for preventing or treating a disease or disorder mediated by endosomal PAR₂ signaling comprising administering to a subject in need thereof an effective amount of a compound of Formula (I) as herein defined, or a pharmaceutically acceptable salt thereof.

In another preferred embodiment, there is provided a compound of Formula (I) as herein defined, or a pharmaceutically acceptable salt thereof, for use in the prophylaxis or treatment of a disease or disorder mediated by PAR₂ signaling.

In a further preferred embodiment, there is provided a compound of Formula (I) as herein defined, or a pharmaceutically acceptable salt thereof, for use in the prophylaxis or treatment of a disease or disorder mediated by endosomal PAR₂ signaling.

In yet another preferred embodiment, there is provided use of a compound of Formula (I) as herein defined, or a pharmaceutically acceptable salt thereof, in the manufacture of a medicament for the prophylaxis or treatment of a disease or disorder mediated by PAR₂ signaling.

Another preferment is directed to use of a compound of Formula (I) as herein defined, or a pharmaceutically acceptable salt thereof, in the manufacture of a medicament for the prophylaxis or treatment of a disease or disorder mediated by endosomal PAR₂ signaling.

The terms “treatment” and “treating” as used herein cover any treatment of a condition or disease in an animal, preferably a mammal, more preferably a human and includes the treatment of any disease or disorder in which inhibition of PAR₂ signaling is beneficial. The terms “prevention” and “preventing” as used herein cover the prevention or prophylaxis of a condition or disease in an animal, preferably a mammal, more preferably a human and includes preventing any disease or disorder in which inhibition of PAR₂ signaling is beneficial.

In a preferred embodiment, the prophylactic or therapeutic method comprises the steps of administering a compound according to the present invention, or a pharmaceutically acceptable salt thereof, to a subject who has a disease or disorder, a symptom of disease or disorder, or predisposition toward a disease or disorder associated with undesired PAR₂ activity as herein described, for the purpose to cure, heal, alleviate, relieve, alter, remedy, ameliorate, improve, or affect the disease or disorder, the symptoms of the disease or disorder, or the predisposition towards the disease or disorder. The prophylactic treatment may reduce the incidence of diseases or disorders associated with undesirable PAR₂ activity.

The prophylactic or therapeutic methods of the present invention may also comprise the administering of a combination of the compounds according to the present invention, or pharmaceutically acceptable salts thereof, to a subject who has a disease or disorder, a symptom of disease or disorder, or predisposition toward a disease or disorder associated with undesired PAR₂ activity as herein described, for the purpose to cure, heal alleviate, relieve, alter, remedy, ameliorate, improve, or affect the disease or disorder, the symptoms of the disease or disorder, or the predisposition towards the disease or disorder. The prophylactic treatment may reduce the incidence of diseases or disorders associated with undesirable PAR₂ activity. In some embodiments, combinations of compounds of the present invention or pharmaceutically acceptable salts thereof may provide enhanced inhibition of PAR₂ activity in comparison to prophylactic or therapeutic methods that utilise only one of the compounds of the present invention or pharmaceutically acceptable salts thereof.

It will also be appreciated by one skilled in the art that the prophylactic or therapeutic methods as herein described could be used in any number of combinations with other treatment modalities currently employed in the art.

Conditions in which PAR₂ expression and/or activity is increased and where it is desirable to reduce said activity, may be identified by those skilled in the art by any or a combination of diagnostic or prognostic assays known in the art, for example, a biological sample obtained from a subject (e.g., blood, serum, plasma, urine, saliva, cerebrospinal fluid, adipose tissue, brain tissue and/or cells derived there from) may be analyzed for PAR₂ expression and/or activity. Such conditions include, but are not limited to, acute and chronic inflammatory disorders, tumour metastasis, gastrointestinal motility, pain, itch, skin disorders such as topic dermatitis, diet-induced obesity, asthma, rheumatoid arthritis, periodontitis, inflammatory bowel diseases, irritable bowel syndrome, cancer, fibrotic diseases, metabolic dysfunction, and neurological diseases.

Within the context of the present invention, the term “pain” includes chronic inflammatory pain (e.g., pain associated with rheumatoid arthritis, osteoarthritis, rheumatoid spondylitis, gouty arthritis, and juvenile arthritis); musculoskeletal pain, lower back and neck pain, sprains and strains, neuropathic pain, sympathetically maintained pain, myositis, pain associated with cancer and fibromyalgia, pain associated with migraine, pain associated with cluster and chronic daily headache, pain associated with influenza or other viral infections such as the common cold, rheumatic fever, pain associated with functional bowel disorders such as non-ulcer dyspepsia, non-cardiac chest pain and irritable bowel syndrome, pain associated with myocardial ischemia, post operative pain, headache, toothache, dysmenorrhea, neuralgia, fibromyalgia syndrome, complex regional pain syndrome (CRPS types I and II), neuropathic pain syndromes (including diabetic neuropathy, chemotherapeutically induced neuropathic pain, sciatica, non-specific lower back pain, multiple sclerosis pain, HIV-related neuropathy, post-herpetic neuralgia, trigeminal neuralgia) and pain resulting from physical trauma, amputation, cancer, toxins, or chronic inflammatory conditions. In a preferred embodiment the pain is somatic pain or visceral pain.

In a preferred embodiment, the present invention provides a method for preventing or treating pain associated with irritable bowel syndrome comprising administering to a subject in need thereof an effective amount of a compound of Formula (I) as herein defined, or a pharmaceutically acceptable salt thereof.

It is considered that the above methods are suitable for the prophylactic and therapeutic treatment of any species, including, but not limited to, all mammals including humans, canines, felines, cattle, horses, pigs, sheep, rats and mice, as well as chickens, birds, reptiles, and lower organisms such as bacteria.

The present invention also provides a pharmaceutical composition comprising a therapeutically effective amount of a compound as hereinbefore defined, or a pharmaceutically acceptable salt thereof, together with at least one pharmaceutically acceptable carrier or diluent.

The term “composition” is intended to include the formulation of an active ingredient with encapsulating material as carrier, to give a capsule in which the active ingredient (with or without other carrier) is surrounded by carriers.

While the compounds as hereinbefore described, or pharmaceutically acceptable salts thereof, may be the sole active ingredient administered to the subject, the administration of other active ingredient(s) with the compound is within the scope of the invention. In one or more embodiments it is envisaged that a combination of two or more of the compounds of the invention will be administered to the subject. It is envisaged that the compound(s) could also be administered with one or more additional therapeutic agents in combination. The combination may allow for separate, sequential or simultaneous administration of the compound(s) as hereinbefore described with the other active ingredient(s). The combination may be provided in the form of a pharmaceutical composition.

The term “combination”, as used herein refers to a composition or kit of parts where the combination partners as defined above can be dosed dependently or independently or by use of different fixed combinations with distinguished amounts of the combination partners, i.e., simultaneously or at different time points. The combination partners can then be administered simultaneously or chronologically staggered, that is at different time points and with equal or different time intervals for any part of the kit of parts. The ratio of the total amounts of the combination partners to be administered in the combination can be varied, e.g., in order to cope with the needs of a patient sub-population to be treated or the needs of the single patient which different needs can be due to age, sex, body weight, etc. of the patient.

As will be readily appreciated by those skilled in the art, the route of administration and the nature of the pharmaceutically acceptable carrier will depend on the nature of the condition and the subject to be treated. It is believed that the choice of a particular carrier or delivery system, and route of administration could be readily determined by a person skilled in the art. In the preparation of any formulation containing the active compound care should be taken to ensure that the activity of the compound is not destroyed in the process and that the compound is able to reach its site of action without being destroyed. In some circumstances it may be necessary to protect the compound by means known in the art, such as, for example, micro encapsulation. Similarly the route of administration chosen should be such that the compound reaches its site of action.

Those skilled in the art may readily determine appropriate formulations for the compounds of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

It is envisaged that when the compounds of the invention are designed to control the absorption of the compound across the intestinal lumen and subsequent systemic exposure of the compounds, the preferred route of administration will be oral or enteral administration. For oral and enteral formulations of the present invention the active compound may be formulated with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavor. Of course, any material used in preparing any dosage unit form should be pharmaceutically pure and substantially non-toxic in the amounts employed. In addition, the compounds of the invention may be incorporated into sustained-release preparations and formulations, including those that allow specific delivery of the active peptide to specific regions of the gut.

Liquid formulations may also be administered enterally via a stomach or oesophageal tube. Enteral formulations may be prepared in the form of suppositories by mixing with appropriate bases, such as emulsifying bases or water-soluble bases.

It is envisaged that when the compounds of the invention are intended for targeted delivery to endocytosed PAR₂, the preferred route of administration will be parenteral administration. The compounds as hereinbefore described, or pharmaceutically acceptable salts thereof, may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the active compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the compounds of the invention in the required amount in the appropriate solvent with various of the other ingredients such as those enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

It is also possible, but not necessary, for the compounds of the present invention to be administered topically, intranasally, intravaginally, intraocularly and the like. The compounds of the present invention may also be administered by inhalation in the form of an aerosol spray from a pressurised dispenser or container, which contains a propellant such as carbon dioxide gas, dichlorodifluoromethane, nitrogen, propane or other suitable gas or combination of gases. The compounds may also be administered using a nebuliser.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above, the principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 2000 mg. Expressed in proportions, the active compound may be present in from about 0.25 μg to about 2000 mg/mL of carrier. In the case of compositions containing supplementary active ingredients, the dosages are determined by reference to the usual dose and manner of administration of the said ingredients.

As used herein, the term “effective amount” refers to an amount of compound which, when administered according to a desired dosing regimen, provides the desired therapeutic activity. Dosing may occur once, or at intervals of minutes or hours, or continuously over any one of these periods. Suitable dosages may lie within the range of about 0.1 ng per kg of body weight to 1 g per kg of body weight per dosage. A typical dosage is in the range of 1 μg to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

General strategies for synthesising compounds of Formula (I) are outlined in the following general scheme, the following general synthetic procedures and in the specific embodiments for synthesis of intermediate products.

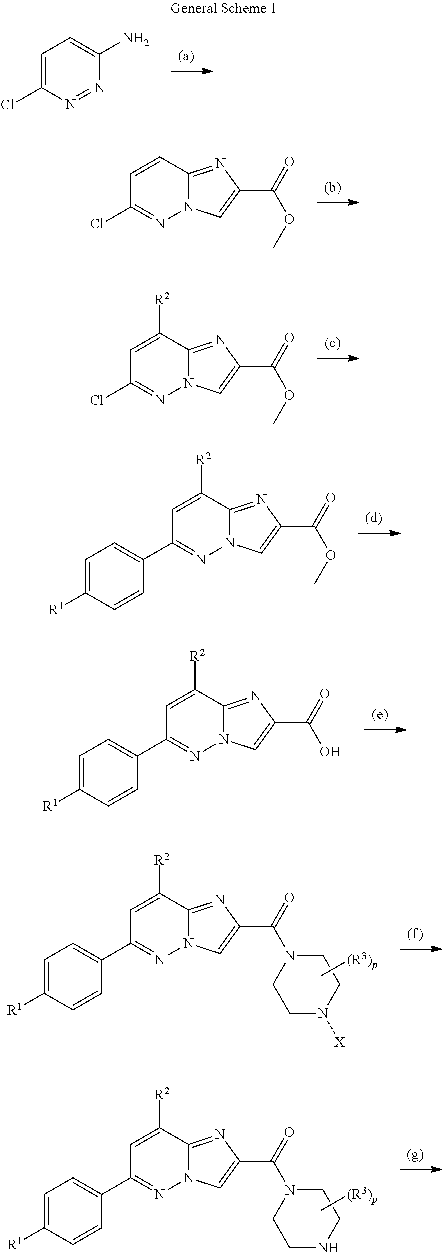

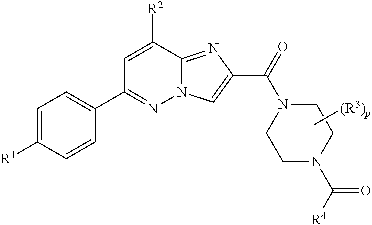

### General Synthetic Procedures:

### General Procedure 1: Amide Formation A

The appropriate carboxylic acid (1.0 equiv) is dissolved in DMF or DMSO (0.15 to 0.3M) before HBTU or HATU (1.1 to 1.5 equiv), the corresponding amine (1.1-1.2 equiv) and DIPEA (2.5 to 3.5 equiv) are added. The mixture is stirred at room temperature between 45 min. and 16 h. Either one of these work-up procedures can be employed:

1) Water is added and the solids are filtered and washed affording the desired product; or
2) Water is added along with EtOAc and the phases are separated. The organic phase is washed 2 other times with water and brine (1:1 mixture), dried over MgSO₄, filtered, and evaporated under reduced pressure. The product is purified on silica gel and/or preparative HPLC.

### General Procedure 2: Amide Formation B

The appropriate carboxylic acid (1.0 equiv), the corresponding amine (1.1-1.2 equiv) and DIPEA (2.5 to 3.5 equiv) are dissolved in DMF or DMSO (0.15 to 0.3M) before PyBOP or PyOxim (1.1 to 1.5 equiv) is added. The mixture is stirred at room temperature between 45 min. and 16 h. Either one of these work-up procedures can be employed:

1) Water is added and the solids are filtered and washed affording the desired product; or
2) Water is added along with EtOAc and the phases are separated. The organic phase is washed 2 other times with 1M HCl and then saturated bicarbonate solutions, dried over MgSO₄, filtered and evaporated under reduced pressure. The product is purified on silica gel and/or preparative HPLC.

### General Procedure 3: Solid Phase A

NH₂I-PEG₁₂-Asp(OChol)-Resin:

Synthesis of the spacer-lipid conjugate is prepared by manual peptide synthesis with standard Fmoc chemistry on NovaSyn® TG^{R} R resin (loading 0.18 mmol/g from NovaBiochem). Coupling of the Fmoc-Asp(OChol)-OH (1.5 equiv) with (1H-Benzotriazol-1-yloxy)(tri-1-pyrrolidinyl)phosphonium hexafluoro-phosphate (PyBOP, 2 equiv) in dichloromethane (DCM) with activation in situ using diisopropylethylamine (DIPEA, 3 equiv) for 3 h. Fmoc deprotection is achieved using 20% piperidine in N,N-dimethylformamide (DMF). Fmoc-PEG₁₂-OH (2 equiv) is coupled to resin-bound NH₂-Asp(OChol) with PyBOP (2 equiv) and DIPEA (3 equiv) in DCM. Fmoc deprotection is achieved using 20% piperidine in N,N-dimethylformamide (DMF). Following final deprotection the antagonists are coupled to the spacer-lipid conjugate on resin. The acid (2-3 equiv) is coupled to resin-bound NH₂-PEG₁₂-Asp(OChol)-resin (250 mg) with PyBOP (2 equiv) and DIPEA (3 equiv) in DCM overnight. The construct is then cleaved from resin using 95% trifluoroacetic acid and purified by reverse-phase high-performance liquid chromatography (HPLC) (Phenomenex Luna C8 column, Lane Cove, Australia) with 0.1% TFA/H₂O and 0.1% TFA/ACN as solvents, providing the lipidated antagonists as viscous oils.

### General Procedure 4: Solid Phase Synthesis of Lipid Conjugates to Antagonists

### Antagonist-PEG-Spacer-Asp(OChol)-Resin:

Synthesis of the spacer-lipid (PEG₂₋₁₂) conjugate to antagonists, amino acids, and mucic acid were prepared using the standard coupling protocol as described in General Procedure 11._{[Jd1]} Completed lipid conjugates were then cleaved and purified as described in General Procedure 11.

General strategies for synthesising lipid anchor (LA) groups of tripartite compounds of Formula (I) are outlined below.

Synthesis of cholesteryl glycolic acid, 3-cholesterylamine, and cholesteryl glycine are described in the literature (Hussey, S. L. et al., J. Am. Chem. Soc. 2001, 123, 12712-12713; Hussey, S. L. et al., Org. Lett. 2002, 4, 415-418; Martin, S. E. et al., Bioconjugate Chem. 2003, 14, 67-74). Lipid anchors of the formula (IIIa) having an amide, sulfonamide, urea or carbamate functional group at position 3 of the steroid structure can be prepared from 3-cholesterylainine, for example, 3-cholesterylamine can be reacted with succinic anhydride in the presence of DMAP to afford the corresponding succinyl substituted compound. The corresponding sulfonamide can be obtained by reaction of 3-cholesterylamine with chlorosulfonylacetic acid, which can be prepared as described in the literature (Hinman, R. L. and Locatell, L. J. Am. Chem. Soc. 1959, 81, 5655-5658). The corresponding urea or carbamate can be prepared according to literature procedures via the corresponding isocyanate (Knolker, H.-J. T. et al., Angew. Chem. Int. Ed. 1995, 34, 2497; Knolker, H.-J. et al., Synlett 1996, 502; Knolker, H.-J. and. Braxmeier, T. Tetrahedron Lett. 1996, 37, 5861). Intermediates of compound (IIIa) having a phosphate or carboxymethylated phosphate at position 3 of the steroid structure can be prepared as described in the literature (Golebriewski, Keyes, Cushman, Bioorg. Med. Chem. 1996, 4, 1637-1648; Cusinato, Habeler, et al., J. Lipid Res. 1998, 39, 1844-1851; Himber, Missano, et al., J. Lipid Res. 1995, 36, 1567-1585). Lipid anchors of the formula (IIIa) having a thiol at position 3 of the steroid structure can be prepared as described in the literature (J. G. Parkes, J. G. et al., Biochim. Biophys. Acta 1982, 691, 24-29), the corresponding carboxymethylated thiols are obtainable by simple alkylation as described for the corresponding amines and alcohols. Lipid anchors of the formula (IIIa) having a difluoromethylenesulfone derivative at position 3 of the steroid structure can be prepared as described in the literature (Lapiene, J. et al., Bioorg. Med. Chem. Lett. 2004, 14, 151-155). Introduction of various side chains at position 17 of lipid anchors of the formula (IIIa) can be achieved by use of literature protocols starting from dehydroisoandrosterone or pregnenolone (Bergmann, E. D. et al., J. Am. Chem. Soc. 1959, 81, 1239-1243 and references therein). Lipid anchors of the formula (IIIa) which are derived from cholestane are obtainable from the corresponding precursors which are derived from cholesterol by reduction of the 5, 6-double bond using literature protocols, e.g., hydrogenation in the presence of various transition metal catalysts.

Lipid anchors of the formula (IIa) having an oxygen derived substituent at position 3 are prepared in a similar manner as described for the lipid anchors of the formula (IIIa) starting from estrone. Lipid anchors of the formula (IIa) having nitrogen derived substitution at position 3 can be prepared in a similar manner as described for lipid anchors of the formula (III) starting from 3-amino estrone, which can be prepared as described in the literature (Zhang, X. and Sui, Z. Tetrahedron Lett. 2003, 44, 3071-3073; Woo, L. W. L. et al., Steroid Biochem. Molec. Biol. 1996, 57, 79-88). Lipid anchors of the formula (IIa) having a sulfur derived substituent at position 3 can be prepared in a similar manner as described for lipid anchors of the formula (III) starting from 3-thioestrone, which can be prepared as described in the literature (Woo, L. W. L. et al., J. Steroid Biochem. Molec. Biol. 1996, 57, 79-88). Introduction of various side chains at position 17 of the estrone structure can be achieved by a Wittig approach, followed by hydrogenation of the resulting double bond as described in the literature (Peters, R. H. et al., J. Org. Chem. 1966, 31, 24-26). Further manipulations within the side chain (e.g., double bond constructions, cycloalkyl decorations) can be achieved by standard protocols (Suzuki couplings, etc.).

Lipid anchors of the formula (Va) belonging to the class of ceramides, dehydroceramides and dihydroceramides with different hydrocarbon groups are obtainable as outlined in the literature (A. H. Merrill, Jr., Y. A. Hannun (Eds.), Methods in Enzymology, Vol. 311, Academic Press, 1999; Koskinen, P. M and Koskinen, A. M. P. Synthesis 1998, 1075). In particular, sphingosine base can be used as key intermediate for all lipid anchors of the formula (Va) having oxygen derived substitution at position 1 of the sphingosine backbone. The corresponding amino derivatives are obtainable by substitution of the sulfonates, which can be prepared from the alcohols according to known protocols. Alkylation and acylation of 1-amino or 1-hydroxy derivatives can be achieved by reaction with bromo acetic acid and succinic anhydride, respectively. The thioacetylated derivative can be prepared by substitution of a sulfonate with mercapto acetic acid. Phosphate and sulfate derivatives are obtainable as described in the literature (A. H. Merrill, Jr., Y A A. Hannun (Eds.), Methods in Enzymology, Vol. 311, Academic Press, 1999; Koskinen, P. M. and Koskinen, A. M. P. Synthesis 1998, 1075). Acylation, sulfonylation, urea and carbamate formation can be achieved by standard procedures. Lipid anchors of the formula (Va) wherein R⁵ is an amino or amino derived function can be prepared starting from sphingosine base, which is available as published by Koskinen (Koskinen, P. M. and Koskinen, A. M. P. Synthesis 1998, 1075), using standard protocols. The corresponding 2-oxygen substituted sphingolipids can be obtained by a strategy published by Yamanoi (Yamanoi, T. et al., Chem. Lett. 1989, 335). Lipid anchors of the formula (Va), wherein both R⁸ represent a hydroxy group, are obtainable by bishydroxylation of the corresponding alkene using known protocols. The corresponding monohydroxy derivatives can be prepared as described in the literature (Howell, A. R. and Ndakala, A. J. Curr. Org. Chem. 2002, 6, 365-391). Modification of substituents R⁶ and R⁹ in lipid anchors of the formula (Va) can be achieved by protocols and strategies outlined in various review articles (Harwood, H. J. Chem. Rev. 1962, 62, 99-154; Gensler, W. J. Chem. Rev. 1957, 57, 191-280).

Lipid anchors of the formula (VIa) are obtainable by protocols described in the literature (M\ddot{υ}ller, S. et al., J. Prakt. Chem. 2000, 342, 779) and by combinations thereof with protocols described for the preparation of lipid anchors of the formula (Va).

Lipid anchors of the formula (VIIa), wherein R⁴ and R⁵ are oxygen derived substituents, can be prepared starting from commercially available (R)-(−)-2,2-dimethyl-1,3-dioxolane-4-methanol as outlined by Fraser-Reid (Schlueter, U. Lu, J. and Fraser-Reid, B. Org. Lett. 2003, 5, 255-257). Variation of substituents R⁶ in compounds of formula (VIIa) can be achieved by protocols and strategies outlined in various review articles (Harwood, H. J. Chem. Rev. 1962, 62, 99-154; Gensler, W. J. Chem. Rev. 1957, 57, 191-280). Lipid anchors of the formula (VIIa), wherein R⁴ and R⁵ are nitrogen derived substituents, are obtainable either starting from the corresponding oxygen substituted systems by nucleophilic replacement of the corresponding sulfonates and further modifications as outlined above, or starting from 1,2,3-triaminopropane which is obtainable as described in the literature (Henrick, K. et al., J Chem. Soc. Dalton Trans. 1982, 225-227).

Lipid anchors of the formula (VIIIa) are obtainable in a similar fashion as lipid anchors of the formula (VIa) or alternatively by ring closing metathesis of Ω-ethenylated intermediates of lipid anchors of the formula (VIIa).

Lipid anchors of the formulae (IXa) and (Xa) are obtainable by synthetic strategies described in the literature (Xue, J. and Guo, Z. Bioorg. Med. Chem. Lett. 2002, 12, 2015-2018; Xue, J. and Guo, Z. J. Am. Chem. Soc. 2003, 16334-16339; Xue, J. et al., J. Org. Chem. 2003, 68, 4020-4029; Shao, N., Xue, J. and Guo, Z. Angew. Chem. Int. Ed. 2004, 43, 1569-1573) and by combinations thereof with methods described above for the preparation of lipid anchors of the formulae (Va) and (VIIa).

Lipid anchors of the formulae (XIa), (XIIa) and (XIIIa) are obtainable by total synthesis following synthetic strategies described in the literature (Knolker, H.-J. Chem. Soc. Rev. 1999, 28, 151-157; Knolker, H.-J. and Reddy, K. R. Chem. Rev. 2002, 102, 4303-4427; Knolker, H.-J. and Knoll, J. Chem. Commun. 2003, 1170-1171; Knolker, H.-J. Curr. Org. Synthesis 2004, 1).

Lipid anchors of the formula (XIVa) can be prepared by Nenitzescu-type indole synthesis starting from 4-methoxy-3-methylbenzaldehyde to afford 6-methoxy-5-methylindole. Ether cleavage, triflate formation and Sonogashira coupling leads to the corresponding 6-alkynyl substituted 5-methylindole. Nilsmeier formylation and subsequent nitromethane addition yields the 3-nitro vinyl substituted indole derivative which is subjected to a global hydrogenation resulting in the formation of the 6-alkyl substituted 5-methyltryptamine. Acylation of the amino group using succinyl anhydride completes the preparation.

Methods for the preparation of tripartite compounds as described herein will be apparent to those skilled in the art and will comprise the steps of a) defining the distance between (a) phosphoryl head group(s) or an equivalent head group of the lipid anchor and a binding and/or interaction site of the inhibitor of endosomal PAR₂ signaling; b) selecting a linker which is capable of spanning the distance as defined in (a); and c) bonding the lipid anchor and the inhibitor of endosomal PAR₂ signaling by the linker as selected in (b).

Corresponding working examples for such a method are given herein. The person skilled in the art is in a position to deduce relevant binding sites or interactions sites of a given or potential inhibitor of endosomal PAR₂ signaling and, accordingly, to determine the distance between (a) phosphoryl head group(s) or an equivalent head group of the lipid anchor and a binding and/or interaction site of the inhibitor of endosomal PAR₂ signaling. Such methods comprise, but are not limited to molecular modelling, in vitro and/or molecular-interaction or binding assays (e.g., yeast two or three hybrid systems, peptide spotting, overlay assays, phage display, bacterial displays, ribosome displays), atomic force microscopy as well as spectroscopic methods and X-ray crystallography. Furthermore, methods such as site-directed mutagenesis may be employed to verify deduced interaction sites of a given inhibitor of endosomal PAR₂ signaling or of a candidate inhibitor of endosomal PAR₂ signaling and its corresponding target.

The skilled addressee will understand that the selection of a linker comprises the selection of linkers known in the art as well as the generation and use of novel linkers, for example, by molecular modelling and corresponding synthesis or further methods known in the art. The term “spanning” as used herein with reference to step b) refers to the length of the linker selected to place the inhibitor of endosomal PAR₂ signaling at the correct locus on the a receptor when the lipid anchor forms part of the lipid layer of the endosome.

The skilled addressee is readily in the position to deduce, verify and/or evaluate the lipophilicity of a given tripartite compound as well as of the individual moiety as described herein. Corresponding test assays to determine endosomal GPCR targeting are provided herein in the examples.

The skilled addressee will understand that the purpose of the linker moiety is to connect the lipid anchor to the inhibitor of endosomal PAR₂ signaling in order to allow the inhibitor of endosomal PAR₂ signaling to interact with PAR₂ when the lipid anchor is anchored in the endosome membrane. The lipid anchor and the linker will contain functional groups allowing for the two to be covalently bonded. The nature of the functional group of the lipid anchor is in no way limited and may include, for example, an amine group that forms an amide bond with the linker, or a hydroxyl or carboxylic acid group that forms and ether or ester bond with the linker.

Similarly, the skilled addressee will understand that selection of the functional group at the end of the linker that connects with the inhibitor of endosomal PAR₂ signaling will be dictated primarily by any available functional groups on the inhibitor of endosomal PAR₂ signaling of choice. For example, if the inhibitor of endosomal PAR₂ signaling comprises a free amine or carboxylic acid group, it is envisaged that the functional group of the linker will comprise a complementary carboxylic acid or amine to form an amide bond.

Where the compounds of the present invention require purification, chromatographic techniques such as reversed-phase high-performance liquid chromatography (HPLC) may be used. The peptides may be characterised by mass spectrometry and/or other appropriate methods.

The invention will now be described with reference to the following non-limiting examples:

### Synthesis of Precursors

### Example 1: Synthesis ethyl 6-chloroimidazo[1,2-b]pyridazine-2-carboxylate (Step (a) of General Synthetic Scheme 1)

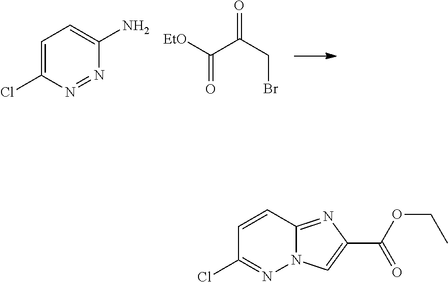

In a 1 L round-bottom flask 6-chloropyridazin-3-amine (30 g, 0.2316 mol) was dissolved in DMF (300 mL). Portionwise addition of ethyl 3-bromo-2-oxo-propanoate (38 mL, 0.3 mol) followed. The mixture was maintained at 50° C. for 1.5 h. The mixture was cooled to room temperature with a water/ice bath and water (600 mL) was added dropwise over 2 h into the reaction mixture. It was then stirred at room temperature overnight. The precipitate formed was filtered off by filtration on Buchner (˜30 min). The precipitate was washed with 3×500 mL of water and dried under vacuum on the Buchner for 2 hrs then 20 hrs in vacuum oven at 40° C. to afford ethyl 6-chloroimidazo[2,1-b]pyridazine-2-carboxylate (29.9 g, 57%) as a yellow solid. ¹H NMR (401 MHz, DMSO) δ 8.85 (s, 2H), 8.27 (d, J=9.6 Hz, 3H), 7.47 (d, J=9.6 Hz, 3H), 4.33 (q, J=7.0 Hz, 6H), 1.32 (t, J=7.1 Hz, 9H).

### Example 2: Synthesis of ethyl 8-tert-butyl-6-chloroimidazo[1,2-b]pyridazine-2-carboxylate (Step (b) of General Synthetic Scheme 1)

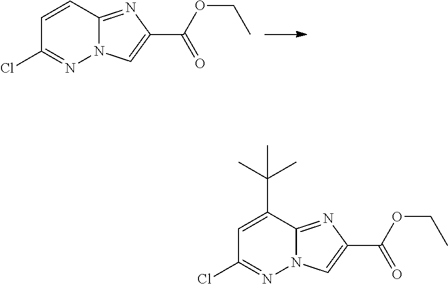 [SMILES: CCOC(=O)c1cn2nc(Cl)ccc2n1 ; CCOC(=O)c1cn2nc(Cl)cc(C(C)(C)C)c2n1]

A 1 L 3 neck round bottom flask equipped with a dropping funnel, a N₂ inlet and condenser was charged with water (98.10 mL) and trifluoroacetic acid (10.72 mL, 139.1 mmol). Once the exotherm finished, ethyl 6-chloroimidazo[2,1-b]pyridazine-2-carboxylate (21 g, 92.72 mmol), 2,2-dimethylpropanoic acid (37.88 g, 21.30 mL, 370.9 mmol) and acetonitrile (200 mL) were added followed by AgN0₃ (7.88 g, 46.36 mmol). The reaction mixture was wrapped in aluminium foil and warmed to 80° C. A solution of ammonium persulfate (35.24 g, 166.9 mmol) in water (98.10 mL) was added via the dropping funnel over 30 min. When addition was completed, the addition funnel was removed and the mixture was equipped with a condenser and heated at 80° C. for 30 minutes.

The reaction was then cooled to room temperature and diluted with 200 mL of ethyl acetate. The filtrate was cooled to 0° C. in an ice/water bath and NH₄OH was added up to pH=8. After 20 min, the mixture was filtered on Celite and washed with ethyl acetate. The layers were separated and the aqueous layer is extracted with 1×200 mL ethyl acetate. The combined organic extracts were washed with 2×200 mL of a solution of 1N NaOH/brine 1:1. The organic phase was filtered on Celite again to remove Ag salts, dried over Na₂SO₄, filtered and concentrated under reduced pressure to afford 35 g of a dark foamy gum.

The crude was chromatographed on silica gel (dichloromethane) to afford ethyl 8-tert-butyl-6-chloro-imidazo[2,1-b]pyridazine-2-carboxylate (7.28 g, 28%) as light yellow solid. ¹H NMR (401 MHz, DMSO) δ 8.81 (s, 1H), 7.17 (s, 1H), 4.35 (q, J=7.1 Hz, 2H), 1.53 (s, 9H), 1.33 (t, J=7.1 Hz, 3H).

### Example 3: Synthesis of ethyl 8-tert-butyl-6-(4-fluorophenyl)imidazo[1,2-b]pyridazine-2-carboxylate (Step (c) of General Synthetic Scheme 1)

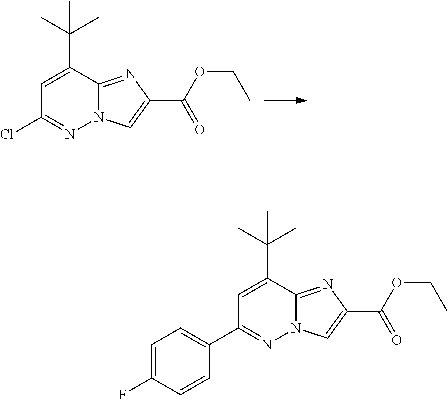 [SMILES: CCOC(=O)c1cn2nc(Cl)cc(C(C)(C)C)c2n1 ; CCOC(=O)c1cn2nc(-c3ccc(F)cc3)cc(C(C)(C)C)c2n1]

To a solution of ethyl 8-tert-butyl-6-chloroimidazo[1,2-b]pyridazine-2-carboxylate (500 mg, 1.755 mmol) in DMF (7 mL) were added (4-fluorophenyl)boronic acid (280 mg, 2.004 mmol), PdCl₂(dppf)₂-DCM (30 mg, 0.03644 mmol) and Na₂CO₃ (1.822 mL of 2 M, 3.644 mmol). After degassing by bubbling N₂ for 5 min, the mixture was heated at 80° C. for 18 h. Water was added along with ethyl acetate and the phases were separated. The organic phase was washed 2 times with water and brine (1:1 mixture), dried over MgSO₄, filtered and evaporated under reduced pressure to afford ethyl 8-tert-butyl-6-(4-fluorophenyl)imidazo[1,2-b]pyridazine-2-carboxylate (545 mg, 90%) as a solid. ¹H NMR (401 MHz, DMSO) δ 8.79 (s, 1H), 8.20-8.11 (m, 2H), 7.51 (s, 1H), 7.46-7.38 (m, 2H), 4.36 (q, J=7.1 Hz, 2H), 1.60 (s, 9H), 1.34 (t, J=7.1 Hz, 3H).

### Example 4: Synthesis of 8-tert-butyl-6-(4-fluorophenyl)imidazo[1,2-b]pyridazine-2-carboxylic acid (Step (d) of General Synthetic Scheme 1)

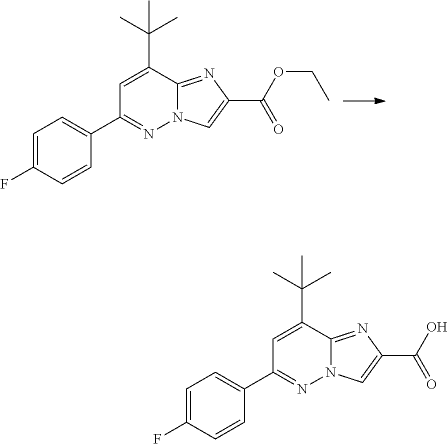 [SMILES: CCOC(=O)c1cn2nc(-c3ccc(F)cc3)cc(C(C)(C)C)c2n1 ; CC(C)(C)c1cc(-c2ccc(F)cc2)nn2cc(C(=O)O)nc12]

Ethyl 8-tert-butyl-6-(4-fluorophenyl)imidazo[1,2-b]pyridazine-2-carboxylate (8.3 g, 24.31 mmol) was dissolved in methanol (388 mL) and NaOH (49 mL of 2.5 M) was added. The solution was stirred at room temperature for 2 h. HCl (6N) was added until acidic pH was reached. Water was then added and a solid precipitated. The solid was washed thoroughly with water and dried to afford 8-tert-butyl-6-(4-fluorophenyl)imidazo[1,2-b]pyridazine-2-carboxylic acid (6.85 g, 90%) as a beige solid. ¹H NMR (401 MHz, DMSO) δ 12.97 (s, 1H), 8.72 (s, 1H), 8.21-8.08 (m, 2H), 7.49 (s, 1H), 7.46-7.34 (m, 2H), 1.60 (s, 9H). LC-MS: 313.97 (M+H+), Retention Time: 3.06

### Example 5: Synthesis of 4-(8-(tert-butyl)-6-(4-fluorophenyl)imidazo[1,2-b]pyridazine-2-carbonyl)-3,3-dimethylpiperazin-1-ium chloride (Steps (e) and (f) of General Synthetic Scheme 1)

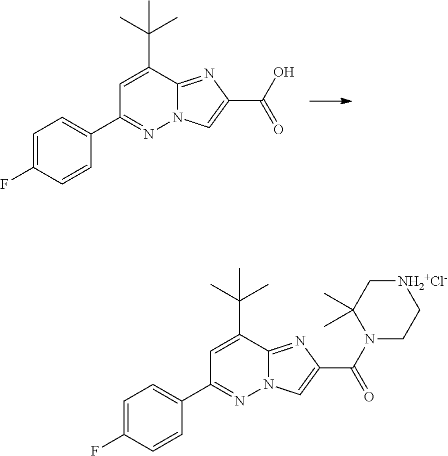

8-tert-butyl-6-(4-fluorophenyl)imidazo[1,2-b]pyridazine-2-carboxylic acid (5 g, 16 mmol), DMF (120 mL), HATU (7.3 g, 19.2 mmol), tert-butyl 3,3-dimethylpiperazine-1-carboxylate (4.1 g, 1.92 mmol) and DIPEA (10 mL, 57.4 mmol) afforded tert-butyl 4-(8-(tert-butyl)-6-(4-fluorophenyl)imidazo[1,2-b]pyridazine-2-carbonyl)-3,3-dimethylpiperazine-1-carboxylate that was dissolved in 4N HCl solution in 1,4-dioxane (60 mL) affording 4-(8-(tert-butyl)-6-(4-fluorophenyl)imidazo[1,2-b]pyridazine-2-carbonyl)-3,3-dimethylpiperazine hydrochloride (6.5 g, 97%) as a solid. ¹H NMR (401 MHz, DMSO) δ 9.71 (bs, 2H), 8.57 (s, 1H), 8.17-8.10 (m, 2H), 7.50 (s, 1H), 7.44-7.36 (m, 2H), 4.16-4.08 (m, 2H), 3.35-3.27 (m, 2H), 3.23-3.14 (m, 2H), 1.61 (s, 6H), 1.59 (s, 9H).

### Example 6: Synthesis of 1-(2-(tert-butoxy)-2-oxoethyl)-1H-1,2,3-triazole-4-carboxylic acid

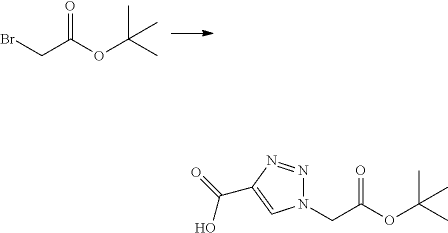

Sodium azide (5 mmol) and tert-butyl bromoacetate (5 mmol) were stirred for 72 h in DMF (10 mL) at room temperature. Propiolic acid (5 mmol) and CuI (0.5 mmol) were added and the stirring continued for additional 48 hrs. The pH of the reaction mixture was adjusted to 4 by addition of 1M HCl and the resultant mixture poured into brine. The aqueous phase was extracted with DCM, dried over MgSO₄, and evaporated to dryness. The crude residue was purified on silica gel to afford the titled product. 29.5%. ¹H NMR (401 MHz, DMSO) δ 8.76-7.63 (m, 2H), 5.42-5.19 (m, 2H), 1.43 (s, 9H). LCMS: Rf=2.90, m/z=225.9 (M−H, C₉H₁₂N₃O₄⁻).

### Synthesis of I-343, Cy5-Cholestanol, and Cy5-Ethyl Ester

### Example 7: Synthesis of (8-(tert-butyl)-6-(4-fluorophenyl)imidazo[1,2-b]pyridazin-2-yl)(2,2-dimethyl-4-(5-methyl-1H-1,2,4-triazole-3-carbonyl)piperazin-1-yl)methanone (I-343)

5-methyl-1H-1,2,4-triazole-3-carboxylic acid (1.2 equiv) was dissolved in DMSO, and then mixed with HATU (1.2 equiv), the corresponding amine (1.0 equiv), and DIPEA (2.5 equiv) (room temperature, overnight). Water was added and the solids were filtered and washed to generate the title product (88% yield). ¹H NMR (400 MHz, DMSO) δ 8.52 (d, J=7.2 Hz, 1), 8.18-8.10 (m, 2), 7.48 (d, J=8.8 Hz, 1H), 7.45-7.36 (m, 2H), 4.34-3.66 (m, 6H), 2.43-2.30 (m, 3H), 1.61 (s, 6H), 1.56 (s, 3H), 1.54 (s, 3H), 1.48 (s, 3H). LCMS: Rf=3.37, m/z=519.3 (M+H, C₂₇H₃₁FN₈O₂⁺).

### Example 8: Synthesis of Cy5-Cholestanol (Cy5-Chol)

Cyanine 5 was conjugated to cholestanol via a flexible PEG linker by standard Fmoc solid-phase peptide synthesis (SPPS) on Fmoc-PAL-PEG-PS resin (Life Technologies, 0.17 mmol/g resin loading). Fmoc deprotection reactions were carried out using 20% v/v piperidine in N,N-dimethylformamide (DMF). Coupling reactions were carried out using Fmoc-protected amino acids with O-(6-chlorobenzotriazol-1-yl)-N,N,N′,N′-tetramethyluronium hexafluorophosphate (HCTU) as coupling agent and N,N-diisopropylethylamine (DIPEA) as activating agent. Cy5-Chol [Cy5-PEG4-PEG3-PEG4-Asp(OChol)-NH₂] was prepared by manual SPPS using Fmoc-Asp(OChol)-OH, Fmoc-PEG4-OH, Fmoc-PEG3-OH, and Fmoc-PEG4-OH as the amino acids. After the final deprotection step, the N-terminus was capped using a mixture of Cy5 acid, HCTU, and DIPEA in DMF, and the peptide construct was then cleaved from resin using 95:2.5:2.5 trifluoroacetic acid (TFA)/triisopropylsilane (TIPS)/water (Jensen, D. D. et al., Sci Transl Med 2017, 9(392): eaal3447).

### Example 9: Synthesis of Cy5-Ethyl Ester

Synthesized using the same procedure as in Example 8, except for the replacement of Fmoc-Asp(OChol)-OH with Fmoc-Asp(OEt)-OH in the first coupling step (Jensen, D. D. et al., Sci Transl Med 2017, 9(392): eaal3447).

### Synthesis of Compounds of the Invention

### Example 10: Synthesis of 3-(4-(8-(tert-butyl)-6-(4-fluorophenyl)imidazo[1,2-b]pyridazine-2-carbonyl)-3,3-dimethylpiperazin-1-yl)-3-oxopropane-1-sulfonic acid (1)

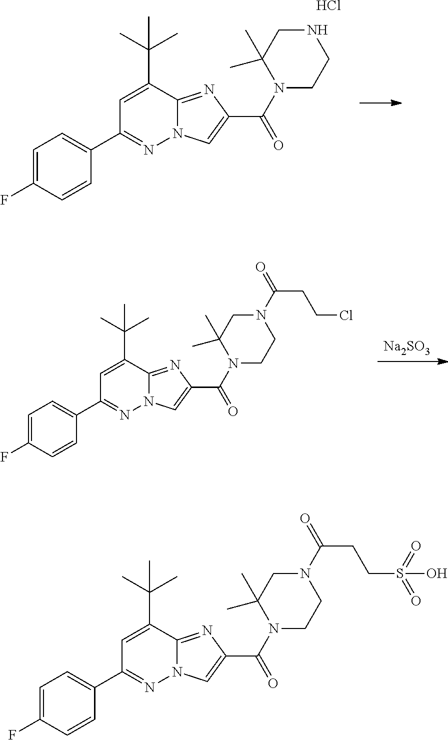

3-Chloropropionic acid (51 mg, 0.472 mmol) was activated with isobutylchloroformate (54 mg, 0.29 mmol) in presence of DIPEA (101 mg, 0.787 mmol) in dry THE at room temperature for 30 minutes. 4-(8-(tert-butyl)-6-(4-fluorophenyl)imidazo[1,2-b]pyridazine-2-carbonyl)-3,3-dimethylpiperazin-1-ium chloride (70 mg, 0.157 mmol) was added and the resultant reaction mixture was stirred for another 60 minutes. The reaction was deemed complete by LCMS, and quenched by addition of saturated sodium bicarbonate. The product was extracted with DCM (3×), dried over MgSO₄ and evaporated to dryness. The crude mixture was redissolved in EtOH (5 mL) and water (5 mL), and Na₂SO₃ (99 mg, 0.787 mmol) added; the mixture was heated at 80° C. overnight. The reaction was deemed complete by LCMS, acidified with TFA and purified on preparative HLPC to provide the titled product (1). 33.7 mg, 39.3% over 2 steps. ¹H NMR (401 MHz, DMSO) δ 8.52 (d, J=4.2 Hz, 1H), 8.18-8.10 (m, 2H), 7.48 (s, 1H), 7.45-7.36 (m, 2H), 4.27-4.21 (m, 2H), 3.74-3.67 (m, 1H), 3.66-3.48 (m, 5H), 2.69-2.62 (m, 1H), 2.62-2.55 (m, 1H), 1.60-1.59 (m, 9H), 1.55 (s, 3H), 1.50 (s, 3H). LCMS (general procedure 13): Rf=3.66, m/z=546.2 (M+H, C₂₆H₃₃FN₅O₅S⁺).

### Example 11: Synthesis of 2-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)acetic acid (2)

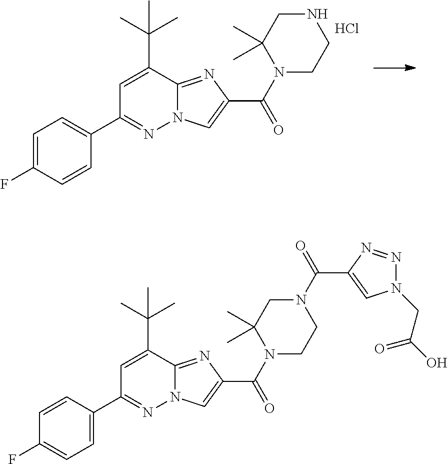 [SMILES: CC(C)(C)c1cc(-c2ccc(F)cc2)nn2cc(C(=O)N3CCNCC3(C)C)nc12 ; Cl ; CC(C)(C)c1cc(-c2ccc(F)cc2)nn2cc(C(=O)N3CCN(C(=O)c4cn(CC(=O)O)nn4)CC3(C)C)nc12]

4-(8-(tert-butyl)-6-(4-fluorophenyl)imidazo[1,2-b]pyridazine-2-carbonyl)-3,3-dimethylpiperazin-1-ium chloride (Example 5) and 1-(2-(tert-butoxy)-2-oxoethyl)-1H-1,2,3-triazole-4-carboxylic acid (Example 6) were used following general procedure 2 to provide the ester intermediate (91.8%), which was deprotected with TFA and DCM to afford the titled product in quantitative yield. LCMS (general procedure 12): R_{f}=3.56, m/z=562.9 (M+H, C₂₈H₃₂FN₈O₄⁺). ¹H NMR (401 MHz, CDCl₃) δ 8.37 (s, 1H), 8.27 (d, J=5.8 Hz, 1H), 7.93 (dd, J=8.8, 5.3 Hz, 2H), 7.24 (d, J=4.1 Hz, 1H), 7.19 (t, J=8.6 Hz, 2H), 5.13-5.07 (m, 2H), 4.60-4.44 (m, 4H), 3.96-3.89 (m, 2H), 1.70 (s, 2H), 1.66 (s, 4H), 1.63-1.58 (m, 9H), 1.49-1.47 (m, 9H). LCMS: Rf=3.71, m/z=619.0 (M+H, C₃₂H₄₀FN₈O₄⁺)

### Example 12: Synthesis of (1S,4R)-4-(4-(8-(tert-butyl)-6-(4-fluorophenyl)imidazo[1,2-b]pyridazine-2-carbonyl)-3,3-dimethylpiperazine-1-carbonyl)-N-((2S,3R,4R,5R)-2,3,4,5,6-pentahydroxyhexyl)cyclohexane-1-carboxamide (3)

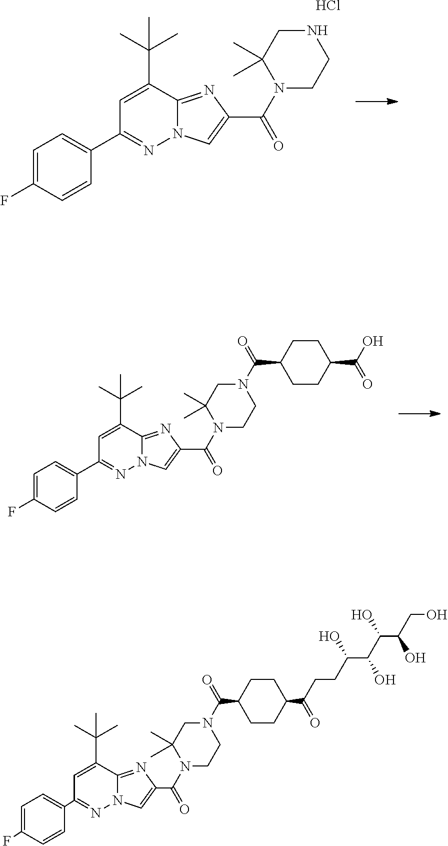 [SMILES: Cl ; CC(C)(C)c1cc(-c2ccc(F)cc2)nn2cc(C(=O)N3CCNCC3(C)C)nc12 ; CC(C)(C)c1cc(-c2ccc(F)cc2)nn2cc(C(=O)N3CCN(C(=O)[C@H]4CC[C@@H](C(=O)O)CC4)CC3(C)C)nc12 ; CC(C)(C)c1cc(-c2ccc(F)cc2)nn2cc(C(=O)N3CCN(C(=O)[C@H]4CC[C@@H](C(=O)CC[C@H](O)[C@@H](O)[C@H](O)[C@H](O)CO)CC4)CC3(C)C)nc12]

4-(8-(tert-butyl)-6-(4-fluorophenyl)imidazo[1,2-b]pyridazine-2-carbonyl)-3,3-dimethylpiperazin-1-ium chloride was coupled to (1S,4S)-4-(methoxycarbonyl)cyclohexane-1-carboxylic acid following general procedure 1. The ester was dissolved in methanol before NaOH 2M (2 equiv) was added. The mixture was stirred at room temperature until completion of hydrolysis. The methanol was removed under reduced pressure and the reaction mixture neutralized by addition of 1M HCl solution. The solids were filtered and washed to obtain the desired acid intermediate. 34%. ¹H NMR (401 MHz, DMSO) δ 12.11 (bs, 1H), 8.51 (d, J=8.5 Hz, 1H), 8.20-8.08 (m, 2H), 7.48 (d, J=4.3 Hz, 1H), 7.46-7.35 (m, 2H), 4.21-4.11 (m, 2H), 3.84-3.45 (m, 4H), 2.61-2.52 (m, 2H), 2.08-1.99 (m, 2H), 1.69-1.42 (m, 21H). LCMS (general procedure 13): R_{f}=3.54, m/z=562.0 (M−H, C₃₁H₃₇FN₅O₄⁻).

The acid intermediate was coupled to D-glucamine following the general procedure 1 to provide the titled product. 48%. ¹H NMR (401 MHz, DMSO) δ 8.51 (d, J=8.4 Hz, 1H), 8.20-8.08 (m, 2H), 7.62-7.45 (m, 2H), 7.45-7.31 (m, 2H), 4.79-4.70 (m, 1H), 4.51-4.20 (m, 4H), 4.22-4.06 (m, 2H), 3.86-3.72 (m, 1H), 3.71-3.52 (m, 5H), 3.52-3.34 (m, 4H), 3.29-3.18 (m, 1H), 3.12-2.97 (m, 1H), 2.77-2.57 (m, 1H), 2.40-2.28 (m, 1H), 2.04-1.84 (m, 2H), 1.81-1.65 (m, 2H), 1.65-1.38 (m, 19H). LCMS: R_{f}=3.28, m/z=727.0 (M+H, C₃₇H₅₂FN₆O₈⁺).

### Example 13: Synthesis of 4-(4-(7-(tert-butyl)-5-(4-fluorophenyl)benzo[d]oxazole-2-carbonyl)-3,3-dimethylpiperazin-1-yl)-4-oxo-N-((2S,3R,4R,5R)-2,3,4,5,6-pentahydroxyhexyl)butanamide (4)

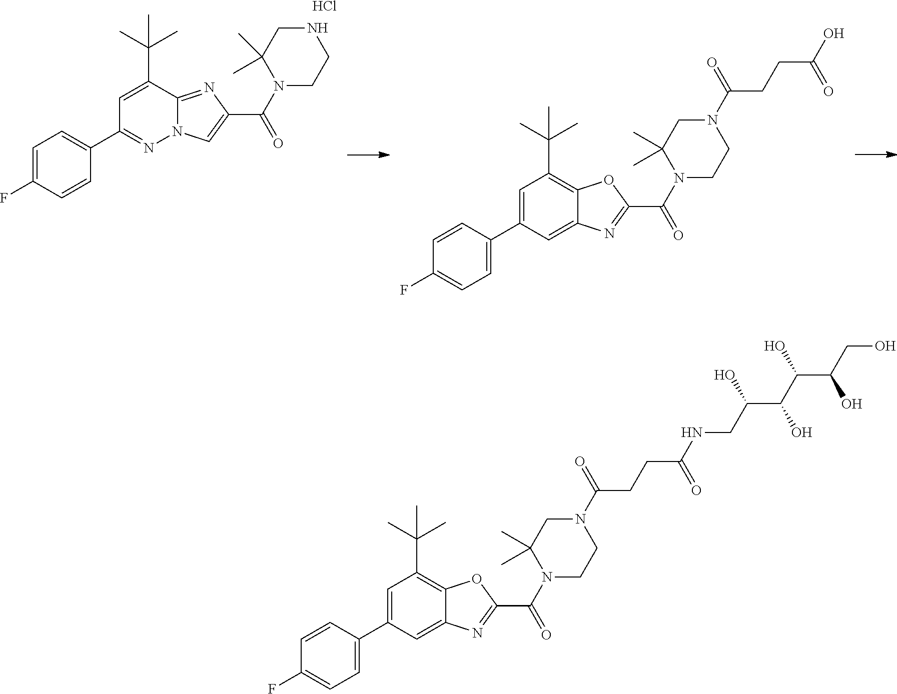 [SMILES: Cl ; CC(C)(C)c1cc(-c2ccc(F)cc2)nn2cc(C(=O)N3CCNCC3(C)C)nc12 ; CC(C)(C)c1cc(-c2ccc(F)cc2)cc2nc(C(=O)N3CCN(C(=O)CCC(=O)O)CC3(C)C)oc12 ; CC(C)(C)c1cc(-c2ccc(F)cc2)cc2nc(C(=O)N3CCN(C(=O)CCC(=O)NC[C@H](O)[C@@H](O)[C@H](O)[C@H](O)CO)CC3(C)C)oc12]

4-(8-(tert-butyl)-6-(4-fluorophenyl)imidazo[1,2-b]pyridazine-2-carbonyl)-3,3-dimethylpiperazin-1-ium chloride (120 mg, 0.27 mmol) was suspended in DCM and succinic anhydride (1.5 equiv) and DIPEA (2 equiv) was added. The reaction mixture was stirred at room temperature for 30 minutes, poured into 1N HCl solution and extracted with DCM. The combined organic phases were dried over MgSO₄, evaporated to dryness and purified on a short silica pad with DCM:MeOH to afford 112.2 mg (81.7%) of 4-(4-(7-(tert-butyl)-5-(4-fluorophenyl)benzo[d]oxazole-2-carbonyl)-3,3-dimethylpiperazin-1-yl)-4-oxobutanoic acid. The acid intermediate was coupled to D-glucamine following the general procedure 1 to provide title product 4. 66%. ¹H NMR (401 MHz, DMSO) δ 8.52 (d, J=7.1 Hz, 1H), 8.19-8.08 (m, 2H), 7.77 (q, J=5.8 Hz, 1H), 7.48 (d, J=2.5 Hz, 1H), 7.45-7.36 (m, 2H), 4.31-4.10 (m, 2H), 3.72 (t, J=5.4 Hz, 1H), 3.68-3.54 (m, 5H), 3.54-3.43 (m, 2H), 3.43-3.33 (m, 2H), 3.26 (dt, J=10.6, 5.7 Hz, 1H), 3.09-2.96 (m, 1H), 2.64-2.51 (m, 2H), 2.38 (dd, J=12.8, 6.7 Hz, 2H), 1.59 (d, J=3.5 Hz, 9H), 1.55 (s, 3H), 1.49 (s, 3H). LCMS (general procedure 13): R_{f}=3.27, m/z=672.9 (M+H, C₂₇H₃₃FN₅O₄⁺).

### Example 14: Synthesis of 2-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-N-(2-(2-(2-hydroxyethoxy)ethoxy)ethyl)acetamide (5)

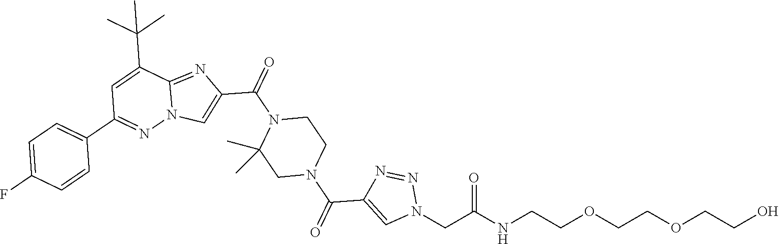

Synthesised via general procedure 2 with the product of Example 11 and 2-(2-(2-aminoethoxy)ethoxy)ethan-1-ol to afford the titled product as a viscous oil. LCMS: R_{f}=3.31, m/z=693.9 (M+H, C₃₄H₄₅FN₉O₆⁺).

### Example 15: Synthesis of 2-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-N-(17-hydroxy-3,6,9,12,15-pentaoxaheptadecyl)acetamide (6)

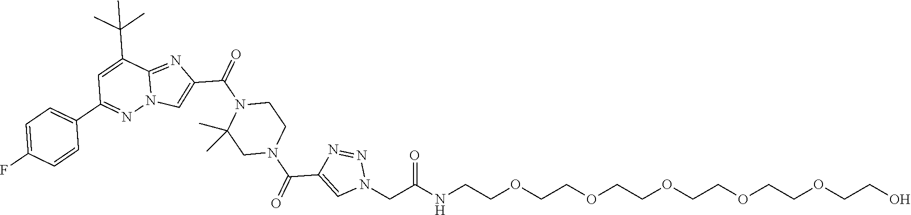

Synthesised via general procedure 2 with the product of Example 11 and 17-amino-3,6,9,12,15-pentaoxaheptadecan-1-ol to afford the titled product as a viscous oil. LCMS: Rf=3.32, m/z=825.8 (M+H, C₄₀H₅₇FN₉O₉⁺).

### Example 16: Synthesis of (2R,3S,4R,5S)—N1-((1s,4S)-4-(4-(8-(tert-butyl)-6-(4-fluorophenyl)imidazo[1,2-b]pyridazine-2-carbonyl)-3,3-dimethylpiperazine-1-carbonyl)cyclohexyl)-2,3,4,5-tetrahydroxyhexanediamide (7)

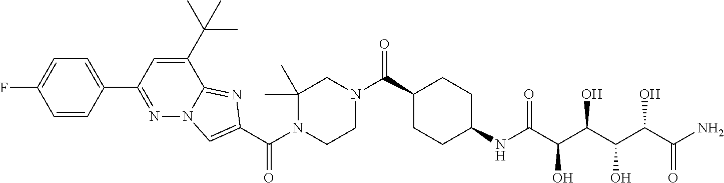 [SMILES: CC(C)(C)c1cc(-c2ccc(F)cc2)nn2cc(C(=O)N3CCN(C(=O)[C@H]4CC[C@@H](NC(=O)[C@H](O)[C@@H](O)[C@@H](O)[C@H](O)C(N)=O)CC4)CC3(C)C)nc12]

Synthesised via general procedure 2 with mucic acid diacetonide-Rink AM resin and (4-((1s,4s)-4-aminocyclohexane-1-carbonyl)-2,2-dimethylpiperazin-1-yl)(8-(tert-butyl)-6-(4-fluorophenyl)imidazo[1,2-b]pyridazin-2-yl)methanone to afford the titled product as an amorphous solid. LCMS: R_{f}=3.36, m/z=709.8 (M+H-NH₂, C₃₆H₄₆FN₆O₈⁺)

### Example 17: Synthesis of methyl 4-(4-(8-(tert-butyl)-6-(4-fluorophenyl)imidazo[1,2-b]pyridazine-2-carbonyl)-3,3-dimethylpiperazine-1-carbonyl)bicyclo[2.2.2]octane-1-carboxylate (8)

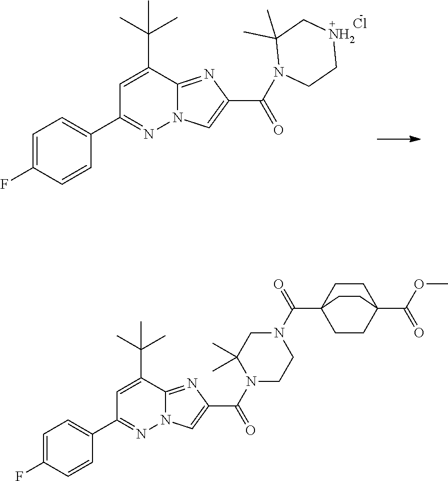 [SMILES: CC(C)(C)c1cc(-c2ccc(F)cc2)nn2cc(C(=O)N3CCNCC3(C)C)nc12 ; Cl ; COC(=O)C12CCC(C(=O)N3CCN(C(=O)c4cn5nc(-c6ccc(F)cc6)cc(C(C)(C)C)c5n4)C(C)(C)C3)(CC1)CC2]

4-(8-(tert-butyl)-6-(4-fluorophenyl)imidazo[1,2-b]pyridazine-2-carbonyl)-3,3-dimethylpiperazin-1-ium chloride (Example 5) and 4-(methoxycarbonyl)bicyclo[2.2.2]octane-1-carboxylic acid were used following general procedure 1 to afford the titled product in 82% yield. ¹H NMR (401 MHz, CDCl₃) δ 8.37 (s, 1H), 7.99-7.89 (m 2H), 7.26 (s, 1H), 7.24-7.17 (m. 2H), 4.46-4.31 (m. 2H), 3.92-3.71 (m, 4H), 3.66 (s, 3H), 2.03-1.79 (m, 12H), 1.64 (s, 3H), 1.61 (s, 9H), 1.56 (s, 3H). LCMS: R_{f}=3.83, m/z=603.9 (M+H, C₃₄H₄₃FN₅O₄⁺).

### Example 18: Synthesis of (3S,10S,13R,17R)-10,13-dimethyl-17-((R)-6-methylheptan-2-yl)hexadecahydro-1H-cyclopenta[a]phenanthren-3-yl (14S)-1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-14-carbamoyl-2,12-dioxo-6,9-dioxa-3,13-diazahexadecan-16-oate (9)

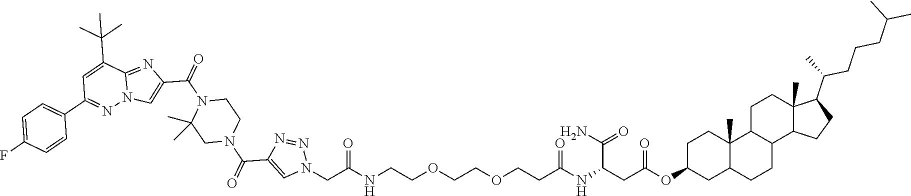

Synthesised via general procedure 4 to afford the titled product as a viscous oil.

LCMS (general procedure 13): Rf=3.18, m/z=1206.56 (M+H, C₆₆H₉₇FN₁₁O₉⁺)

### Example 19: Synthesis of (3S,10S,13R,17R)-10,13-dimethyl-17-((R)-6-methylheptan-2-yl)hexadecahydro-1H-cyclopenta[a]phenanthren-3-yl (44S)-1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-44-carbamoyl-2,42-dioxo-6,9,12,15,18,21,24,27,30,33,36,39-dodecaoxa-3,43-diazahexatetracontan-46-oate (10)

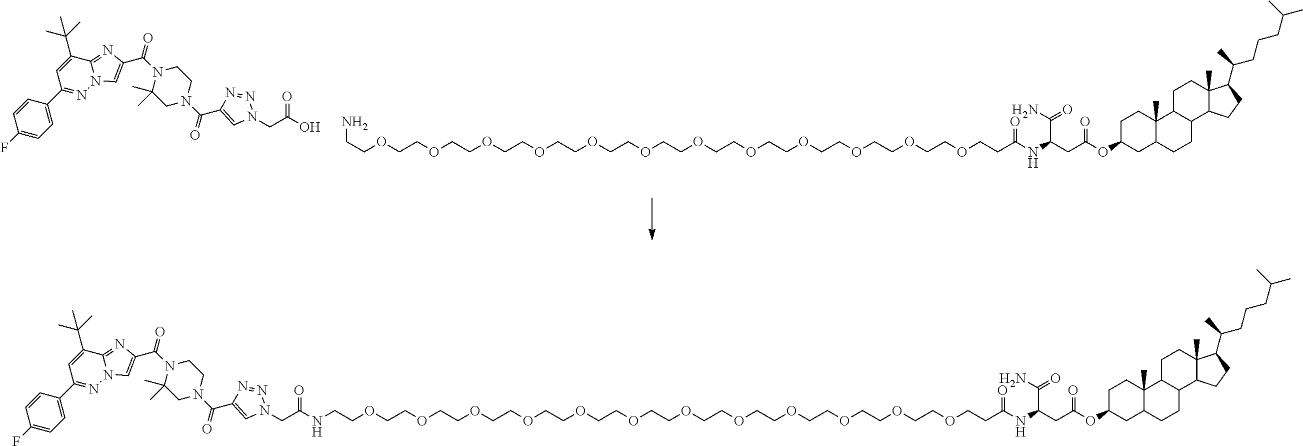

Step 1: Resin-bound NH₂-PEG₁₂-Asp(OChol)-resin was synthesized as per the general procedure 3. The amine was cleaved from resin using 95% trifluoroacetic acid, and evaporated to dryness to afford a crude NH₂—PEG₁₂-Asp(OChol).

Step 2: The acid product of Example 11 and NH₂-PEG₁₂-Asp(OChol) from step 1 were used following the general procedure 2 to provide the titled product (45%). ¹H NMR (401 MHz, CDCl₃) δ 8.43 (d, J=4.2 Hz, 2H), 7.99-7.90 (m, 2H), 7.74-7.50 (m, 3H), 7.34-7.26 (m, 2H), 7.19 (dt, J=19.6, 7.5 Hz, 3H), 5.22 (s, 2H), 4.95-4.86 (m, 1H), 4.74-4.64 (m, 1H), 4.62-4.40 (m, 4H), 4.28-4.17 (m, 1H), 3.97-3.79 (m, 3H), 3.74-3.54 (m, 45H), 3.49 (dd, J=10.9, 5.7 Hz, 2H), 3.03 (dd, J=17.2, 5.1 Hz, 1H), 2.70 (dd, J=17.9, 5.9 Hz, 1H), 2.57 (t, J=3.8 Hz, 2H), 1.96 (dd, J=9.3, 3.1 Hz, 1H), 1.86-1.18 (m, 43H), 1.18-0.78 (m, 26H), 0.67-0.58 (m, 4H). LCMS (high res): m/z=824.5132 (M+2H, C₈₆H₁₃₈FN₁₁O₁₉²⁺).

### Example 20: Synthesis of (3S,10S,13R,17R)-10,13-dimethyl-17-((R)-6-methylheptan-2-yl)hexadecahydro-1H-cyclopenta[a]phenanthren-3-yl (20S)-1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-20-carbamoyl-2,18-dioxo-6,9,12,15-tetraoxa-3,19-diazadocosan-22-oate (11)

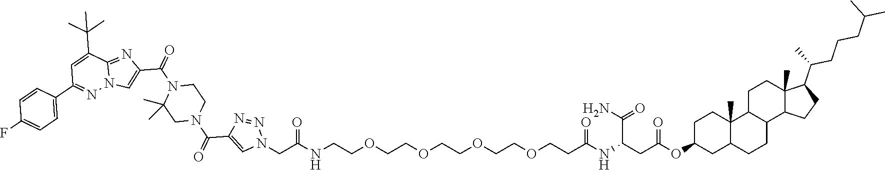

Synthesised via general procedure 4 to afford the titled product as a viscous oil.

LCMS: R_{f}=3.28, m/z=1294.66 (M+H, C₇₀H₁₀₅FN₁₁O₁₁⁺).

### Example 21: Synthesis of (3S,10S,13R,17R)-10,13-dimethyl-17-((R)-6-methylheptan-2-yl)hexadecahydro-1H-cyclopenta[a]phenanthren-3-yl (32S)-1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-32-carbamoyl-2,30-dioxo-6,9,12,15,18,21,24,27-octaoxa-3,31-diazatetratriacontan-34-oate (12)

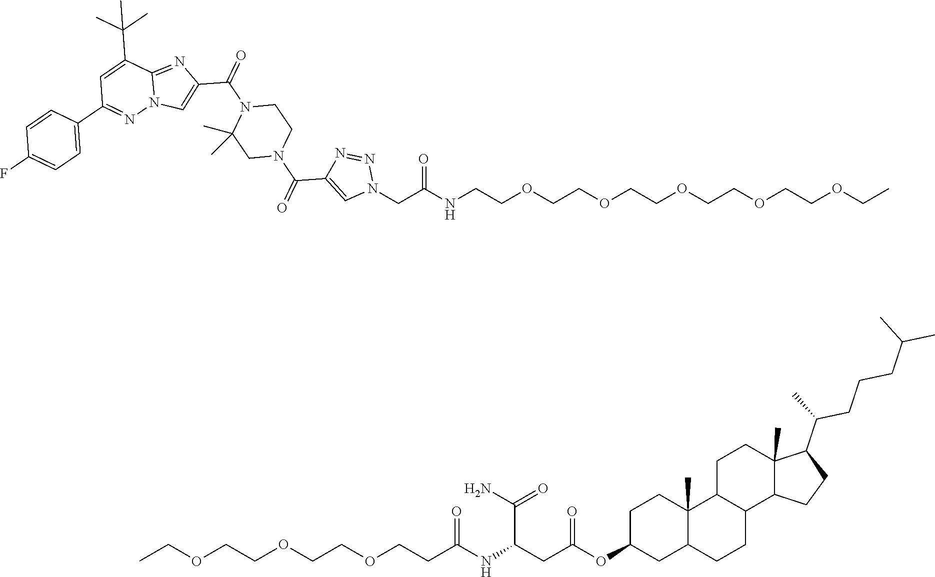

Synthesised via general procedure 4 to afford the titled product as a viscous oil.

LCMS: Rf=2.83, m/z=1470.878 (M+H, C₇₈H₁₂₁FN₁₁O₁₅⁺).

### Example 22: Synthesis of (3S,10S,13R,17R)-10,13-dimethyl-17-((R)-6-methylheptan-2-yl)hexadecahydro-1H-cyclopenta[a]phenanthren-3-yl (37S)-1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-37-carbamoyl-2,7,35-trioxo-11,14,17,20,23,26,29,32-octaoxa-3,8,36-triazanonatriacontan-39-oate (13)

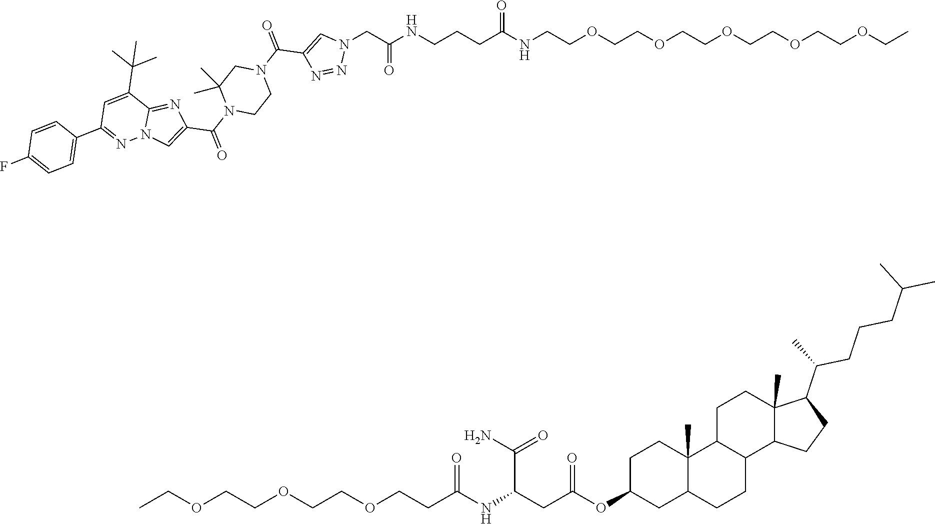

Synthesised via general procedure 4 to afford the titled product as a viscous oil.

LCMS: Rf=2.30, m/z 1555.98 (M+H, C₈₂H₁₂₈FN₁₂O₁₆⁺).

### Example 23: Synthesis of (8R,9S,10S,13R,14S,17R)-10,13-dimethyl-17-((R)-6-methylheptan-2-yl)hexadecahydro-1H-cyclopenta[a]phenanthren-3-yl (26S,51S)-1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-51-carbamoyl-26-(3-guanidinopropyl)-2,24,27,49-tetraoxo-6,9,12,15,18,21,31,34,37,40,43,46-dodecaoxa-3,25,28,50-tetraazatripentacontan-53-oate (14)

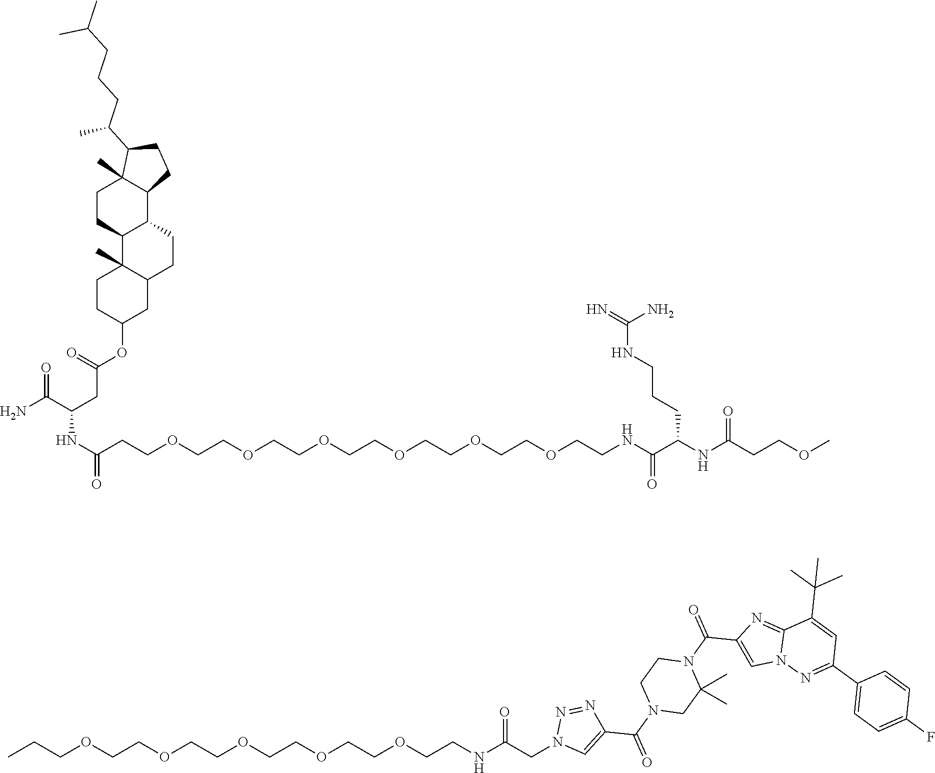

Synthesised via general procedure 4 to afford the titled product as a viscous oil.

LCMS (general procedure 13): Rf=3.44, m/z=936.8 (M+2H, C₉₅H₁₅₅FN₁₆O₂₁²⁺).

### Example 24: Synthesis of (S)-3-(1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-2-oxo-6,9,12,15,18,21,24,27,30,33,36,39-dodecaoxa-3-azadotetracontan-42-amido)-N1-hexadecylsuccinamide (15)

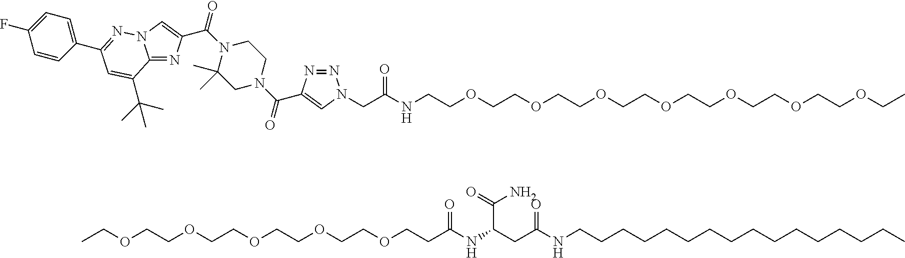

Synthesised via general procedure 4 where Fmoc-L-Asp(OChol)-OH was replaced by Fmoc-L-Asp(NH(CH₂)₁₅CH₃)—OH to afford the titled product as a viscous oil.

LCMS (high res): m/z=750.4622 (M+2H, C₇₅H₁₂₅FN₁₂O₁₈²⁺)

### Example 25: Synthesis of (3S,5R,8R,9S,10S,13R,14S,17R)-10,13-dimethyl-17-((R)-6-methylheptan-2-yl)hexadecahydro-1H-cyclopenta[a]phenanthren-3-yl (3S,28S,29R,30S,31R)-32-((4-(4-(8-(tert-butyl)-6-(4-fluorophenyl)imidazo[1,2-b]pyridazine-2-carbonyl)-3,3-dimethylpiperazine-1-carbonyl)cyclohexyl)amino)-3-carbamoyl-28,29,30,31-tetrahydroxy-5,27,32-trioxo-8,11,14,17,20,23-hexaoxa-4,26-diazadotriacontanoate (16)

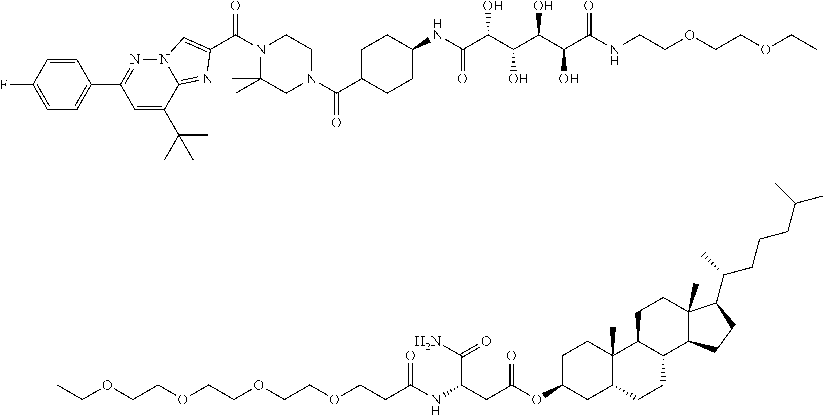

Synthesised via general procedure 4 where prior to coupling to PAR₂ antagonist, mucic acid diacetonide was coupled to PEG spacer. The titled product was isolated as a glass.

LCMS (high res): m/z=1547.9463 (M+H, C₈₂H₁₂₉FN₉O₁₈²⁺).

### Example 26: Synthesis of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((R)-1-(4-(4-(8-(tert-butyl)-6-(4-fluorophenyl)imidazo[1,2-b]pyridazine-2-carbonyl)-3,3-dimethylpiperazine-1-carbonyl)-1H-1,2,3-triazol-1-yl)-44-(hydrazinecarbonyl)-2,42-dioxo-6,9,12,15,18,21,24,27,30,33,36,39-dodecaoxa-3,43-diazaoctatetracontan-48-yl)carbamate (17)

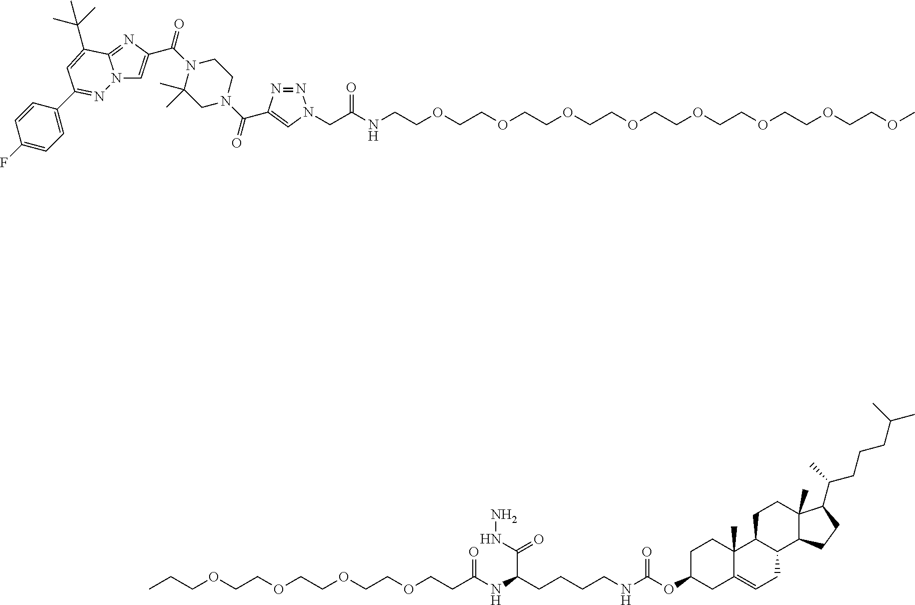

Synthesized via general procedure 4 where Fmoc-D-Lys(NCOChol)-NHNH₂ was replaced by Fmoc-L-Asp(NH(CH₂)₁₅CH₃)—OH to afford the titled product as a viscous oil.

LCMS (high res): m/z=859.5373 (M+2H, C₇₅H₁₂FN₁₂O₁₈²⁺)

### Example 27: Inhibition of PAR₂ in Transfected KNRK or HT29 Cells

KNRK-hPAR2, KNRK or HT-29 cells were seeded at a density of 50×103 cells/well in a clear poly-d-lysine coated 96 well tissue culture plate. Following 24 h incubation at 37° C. and 5% CO₂, media was removed and replaced with 80 μl IP1 stimulation buffer (10 mM HEPES, 1 mM CaCl₂, 0.5 mM MgCl₂, 4.2 mM KCl, 146 mM NaCl, 5.5 mM glucose, 50 mM LiCl). Following stimulation buffer addition, the wells received 10 μl of 10× antagonists or DMSO vehicle. All plates were further incubated at 37° C., 5% CO₂ for 30 min. 10 μl of 2F or ATP was added to the plates and further incubated for 40 min. Following incubation, stimulation buffer was quickly removed by aspiration and replaced with 25 μl lysis buffer (IP-One HTRF® assay kit, Cisbio). Following incubation of the lysates at 37° C., 5% CO₂ for 10′, 10 μl lysate was transferred to a 384-well OptiPlate (PerkinElmer) and detected using the IP-One HTRF® assay kit (Cisbio).

Compounds of the invention (added at 10×concentrations in a 10 μL volume) were incubated for 30 min prior to the addition of 10 μM ATP or 100 nM or 300 nM PAR2 agonist (2F; EC₈₀, added at 10×concentrations in a 10 μL volume) and then further incubated for 40 min. After lysis, the inositol phosphate 1 was quantified with IP-One HTRF® assay kit, Cisbio. The data was analysed with Prism, GraphPad to calculate the IC₅₀ values, shown below in Table 1.

TABLE 1
Inhibitory potencies (pIC₅₀) for the compounds of the invention in an IP₁
accumulation assay (^{a}) or a Ca²⁺ FLIPR^{TETRA} assay (^{b}).

| | KNRK-PAR₂ | HT-29 | |
|---|---|---|---|
| | 100 nM 2F | 300 nM 2F | |
| Compound | pIC₅₀ ± SEM | pIC₅₀ ± SEM | |
| No. | (IC₅₀, nM) | (IC₅₀, nM) | |
| 1 | ND^{c} | 5.23 ± 0.07 | (5900) |
| 2 | ND | 6.86 ± 0.11 | (140) |
| 3 | <5.00 (<10000) | 7.39 ± 0.11 | (40) |
| 4 | ND | <5.00 | (<10000) |
| 5 | ND | 6.745 ± 0.22 | (180) |
| 6 | ND | 6.013 ± 0.21 | (971) |
| 7 | ND | 6.392 ± 0.23 | (405) |
| 8 | <5.00 (<10000) | 7.37 ± 0.12 | (43) |
| 9 | ND | 6.15 ± 0.21 | (700) |
| 10 | ND | 6.18 ± 0.07 | (670) |
| 11 | ND | 6.01 ± 0.28 | (970) |
| 12 | ND | 6.08 ± 0.22 | (827) |
| 13 | ND | 6.133 ± 0.31 | (736) |
| 14 | ND | 5.81 ± 0.35 | (1547) |
| 15 | ND | 7.326 ± 0.30 | (47) |
| 16 | ND | 5.685 ± 0.30 | (2066) |
| 17 | ND | 5.249 ± 0.26 | (5632) |

^{c}ND, less than 50% inhibition of 2F was observed when 30 μM of antagonist was used

As is evident from the above results, the compounds of the invention are effective inhibitors of PAR₂ signaling.

### Example 28: PAR₂-Mediated Nociception

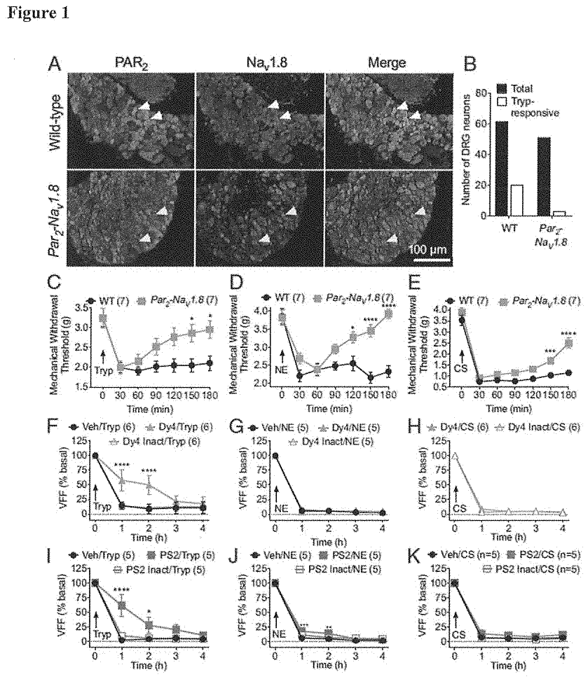

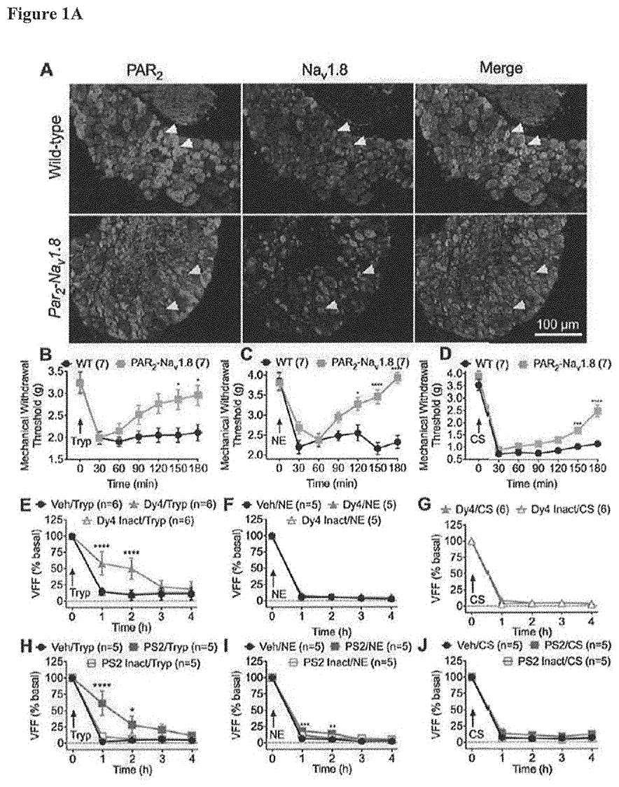

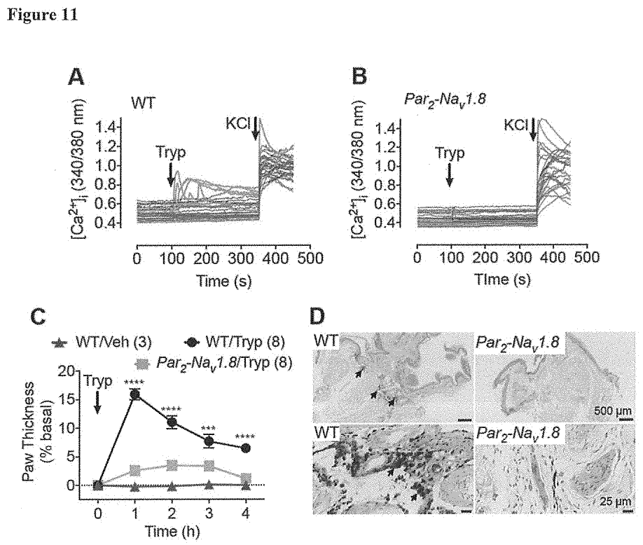

Proteases may induce pain by activating PAR₂ on nociceptors or other cell types. To determine the contribution of PAR₂ on nociceptors, mice were bred expressing PAR₂ flanked by LoxP sites (Par₂^{lox/lox}) with mice expressing Cre recombinase targeted to nociceptors using the Na_{V}1.8 promoter (Scn10a) (Stirling L. C. et al., Pain 2005, 113(1-2): 27-36). Par₂-Na_{V}1.8 mice lacked immunoreactive PAR₂ in Na_{V}1.8-positive nociceptors (FIG. 1A). Whereas 31% (20 of 65) of small-diameter (<25 μm) DRG neurons from WT mice responded to trypsin (100 nM) with increased [Ca²⁺]ᵢ, only 6% (3 of 51) of neurons from Par₂-Na_{V}1.8 mice responded (FIGS. 1B, 11A, and 11B). Nociception was assessed by measuring withdrawal responses to stimulation of the plantar surface of the hindpaw with von Frey filaments. In WT mice, intraplantar injection (10 μl) of trypsin (80 nM), NE (3.9 μM) or CS (5 μM) induced mechanical allodynia within 30 min, that was maintained for 180 min (FIG. 1B-D). In Par₂-Na_{V}1.8 mice, the initial responses were maintained, but responses after 120 min were diminished. At 180 min, when mechanical allodynia in WT mice was fully maintained, responses in Par₂-Na_{V}1.8 mice had returned to baseline (NE) or were significantly attenuated (trypsin, CS). In WT mice, intraplantar trypsin increased paw thickness-measured using calipers-which peaked at 1 h and was maintained for 4 h, and stimulated an influx of neutrophils after 4 h, consistent with inflammation (FIGS. 11C and 11D). Trypsin-induced inflammation was markedly diminished in Par₂-Na_{V}1.8 mice.

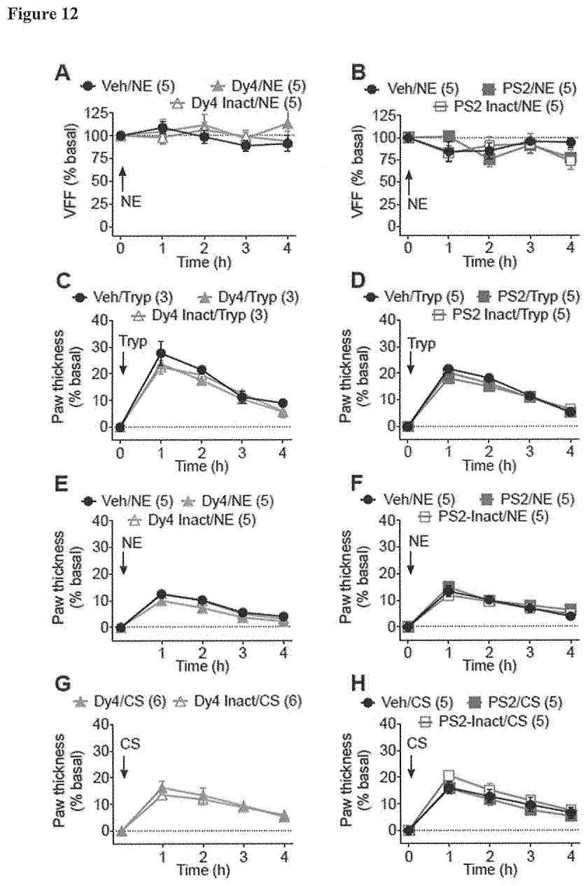

To assess the contribution of endocytosis to protease-induced nociception, Dyngo4a (Dy4, dynamin inhibitor; Robertson M. J. et al., Nat. Protoc. 2014, 9(4): 851-870), PitStop2 (PS2, clathrin inhibitor; Robertson, M. J., et al., Nat. Protoc. 2014, 9(7): 1592-1606), inactive (inact) analogs (50 μM), or vehicle (0.2% DMSO, 0.9% NaCl) (10 μl) was administered by intraplantar injection to mice. After 30 min, trypsin (10 nM), NE (1.2 μM), or CS (2.5 μM) (10 μl) was injected into the same paw. In controls (vehicle or inactive analogues), trypsin, NE and CS induced mechanical allodynia for 4 h (FIG. 1E-J). Dy4 and PS2 inhibited trypsin-induced allodynia at 1 and 2 h (FIG. 1E, H), whereas NE- (FIG. 1F, I) and CS- (FIG. 1G, J) induced allodynia was unchanged or minimally affected. Endocytic inhibitors or proteases did not influence withdrawal responses of the non-injected contralateral paw (FIG. 12A, B). Trypsin, NE and CS increased paw thickness, consistent with edema (FIG. 12C-H). Dynamin and clathrin inhibitors did not affect edema.

The results suggest that proteases induce persistent nociception by activating PAR₂ on Na_{V}-1.8 nociceptors, and that PAR₂ endocytosis is necessary for the nociceptive actions of trypsin, but not NE or CS.

### Example 29: PAR₂-Mediated Hyperexcitability of Nociceptors

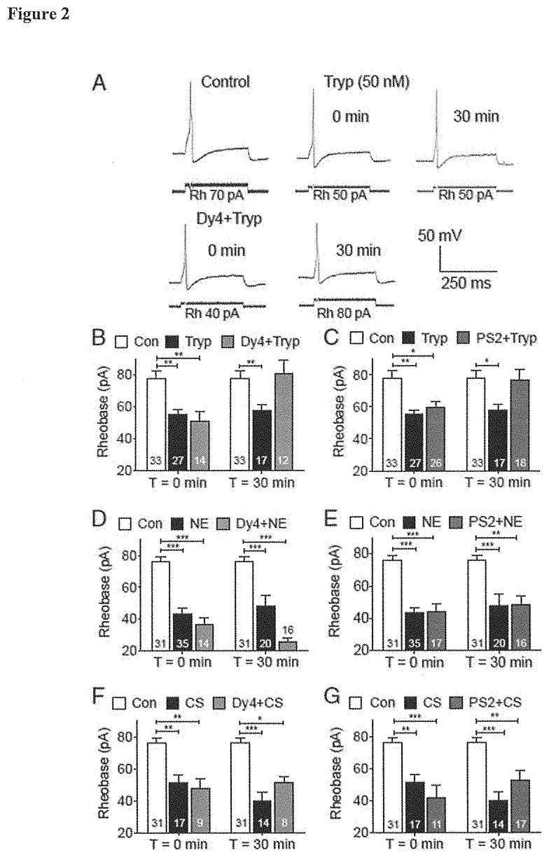

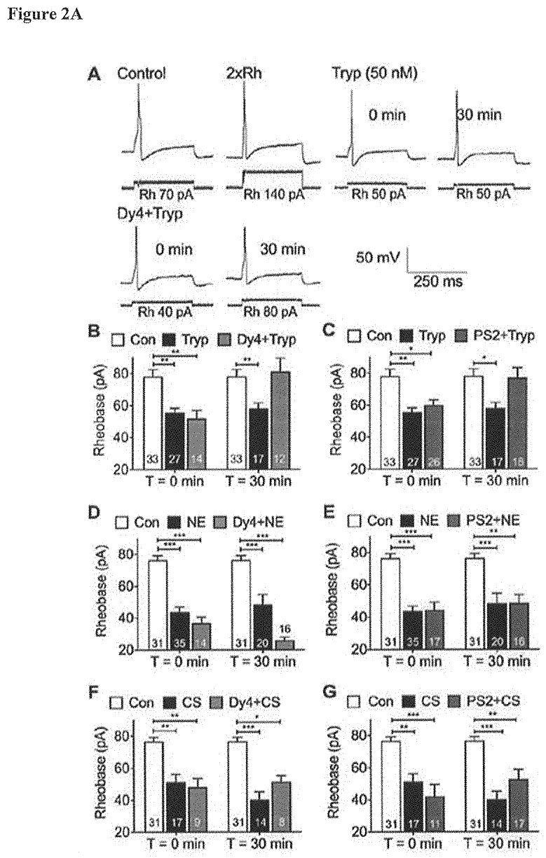

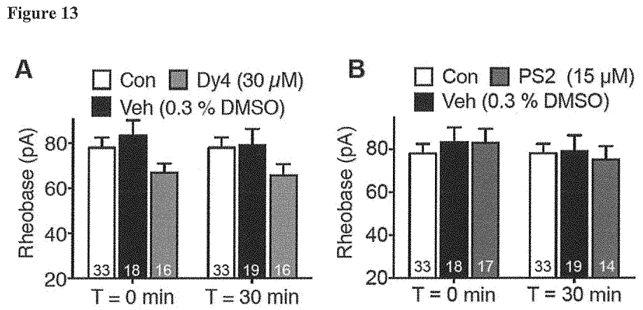

To evaluate the contribution of endocytosis to protease-induced hyperexcitability of nociceptors, the rheobase (minimal current to fire one action potential) of small diameter neurons of mouse dorsal root ganglia (DRG) was measured by patch clamp recording. Neurons were preincubated with trypsin (50 nM, 10 min), NE (390 nM, 30 min), CS (500 nM, 60 min) (conditions selected to cause robust hyperexcitability), or vehicle, and washed. Rheobase was measured 0 or 30 min after washing. Trypsin, NE and CS decreased rheobase at 0 and 30 min, indicating an initial hyperexcitability that is maintained for at least 30 min (FIG. 2). Dy4 (30 μM) or PS2 (15 μM) did not affect the capacity of trypsin, NE or CS to cause initial hyperexcitability (0 min). Dy4 and PS2 abolished the persistent effects of trypsin (FIG. 2A-C), but not of NE (FIG. 2D, E) or CS (FIG. 2F, G) (30 min). Dy4, PS2, or vehicle (0.3% DMSO) did not affect the basal excitability of DRG neurons (FIG. 13).

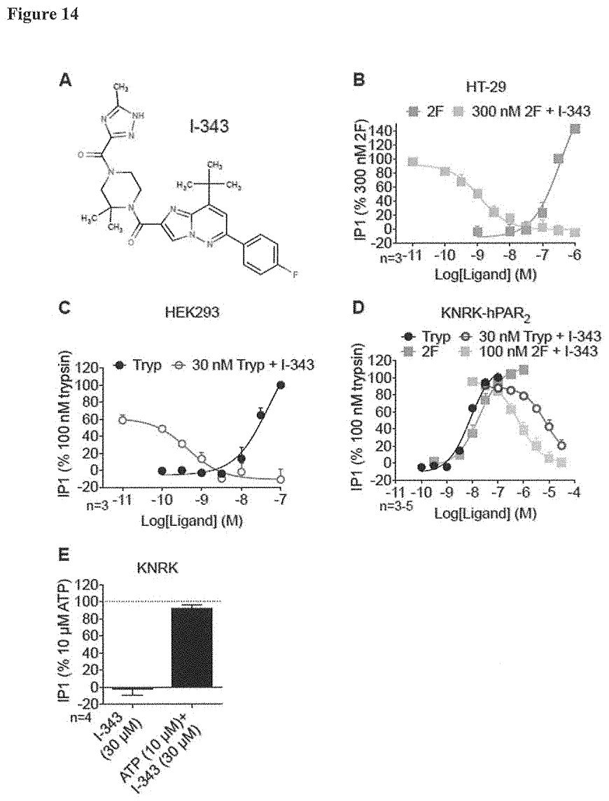

Inhibition of PAR₂ signaling with known PAR₂ inhibitor I-343 (FIG. 14A) was investigated in HT-29 cells and HEK293 cells, which express endogenous PAR₂, and in KNRK cells expressing human (h) PAR₂. Accumulation of inositol phosphate-1 (IP1) was measured in response to the PAR₂-selective agonist 2-Furoyl-LIGRLO-NH₂ (2F), an analogue of the trypsin-exposed tethered ligand, or trypsin. I-343 inhibited 2F (300 nM)-induced IP₁ in HT-29 cells (pIC₅₀ 8.93±0.11, IC₅₀ 1.1 nM) and 2F (100 nM)-induced IP₁ in KNRK-hPAR₂ cells (pIC₅₀ 6.18±0.11, IC₅₀ 666 nM; FIG. 14B-D). I-343 inhibited trypsin (30 nM)-induced IP₁ in HEK293 cells (pIC₅₀ 9.36±0.20, IC₅₀ 0.4 nM) and in KNRK-hPAR₂ cells (pIC₅₀ 5.13±0.14, IC₅₀ 7507 nM). I-343 did not affect ATP (10 μM)-stimulated IP₁ in KNRK cells (FIG. 14E).

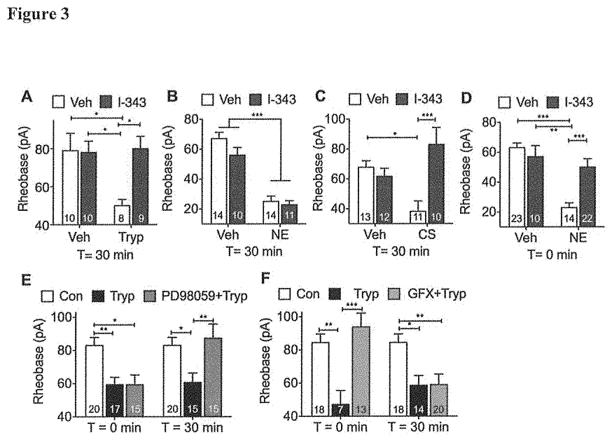

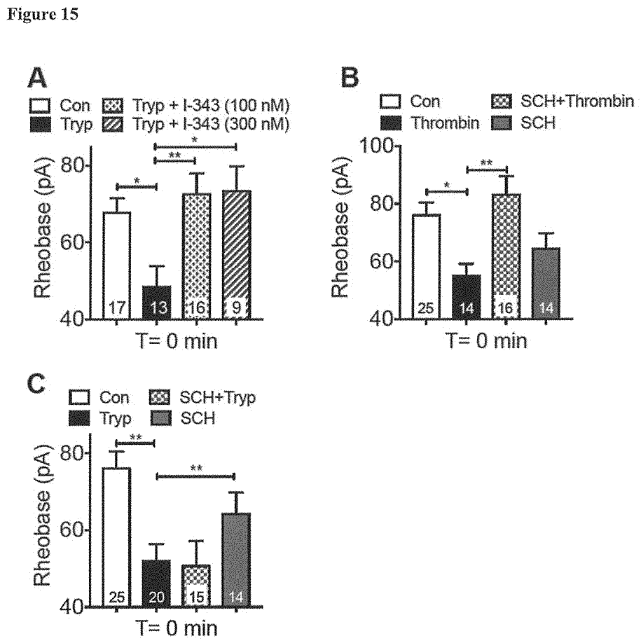

I-343 (10 μM) prevented the decrease in rheobase 30 min after trypsin and CS, but not NE (FIG. 3A-C). However, I-343 prevented the decrease in rheobase 0 min after NE (FIG. 3D). I-343 (100 nM, 300 nM) also prevented the decrease in rheobase 0 min after trypsin (FIG. 15A). When neurons were incubated with thrombin (50 nM, 20 min) and washed, there was an immediate decrease in rheobase that was prevented by preincubation with the PAR₁ antagonist SCH79797 (1 μM, 10 min; Ahn, H. S., et al., Biochem. Pharmacol. 2000, 60(10): 1425-1434); SCH79797 alone had no effect (FIG. 15B) and SCH79797 did not affect the response to trypsin (FIG. 15C). Thus, PAR₂ mediates the persistent actions of trypsin and CS, and the initial effects of NE, but NE causes persistent hyperexcitability by a different mechanism. Another PAR₂ antagonist, GB88, also prevents trypsin, NE and CS activation of nociceptors (Lieu, T. et al. Br J Pharmacol 2016, 173(18): 2752-2765). Trypsin-activated PAR₂ signals from endosomes by βARR- and Raf-1-dependent processes, which activate ERK (DeFea, K. A. et al., J Cell Biol 2000, 148(6): 1267-1281). PD98059 (50 μM), which inhibits activation of mitogen-activated protein kinase kinase 1 (MEK1) (Lieu, T. et al., Br J Pharmacol 2016, 173(18): 2752-2765) did not affect initial trypsin-induced hyperexcitability, but prevented persistent trypsin-induced hyperexcitability (FIG. 3E). In contrast, GF109203X (Bis-1, 10 μM), which inhibits PKCα and other kinases (Davies, S. P. et al., Biochem J 2000, 351(Pt 1): 95-105), prevented the initial but not the persistent effects of trypsin (FIG. 3F).

Thus, trypsin induces initial hyperexcitability of nociceptors by PAR₂/PKC signaling from the plasma membrane, and persistent hyperexcitability by PAR₂/ERK signaling from endosomes. Adenylyl cyclase and PKA mediate NE- and CS-induced hyperexcitability (Zhao, P. et al., J. Biol. Chem. 2014, 289(39): 27215-27234; Zhao, P. et al., J. Biol. Chem. 2015, 290(22): 13875-13887) of nociceptors, which was not further studied.

### Example 30: PAR₂ Endocytosis and Compartmentalized Signaling in Nociceptors

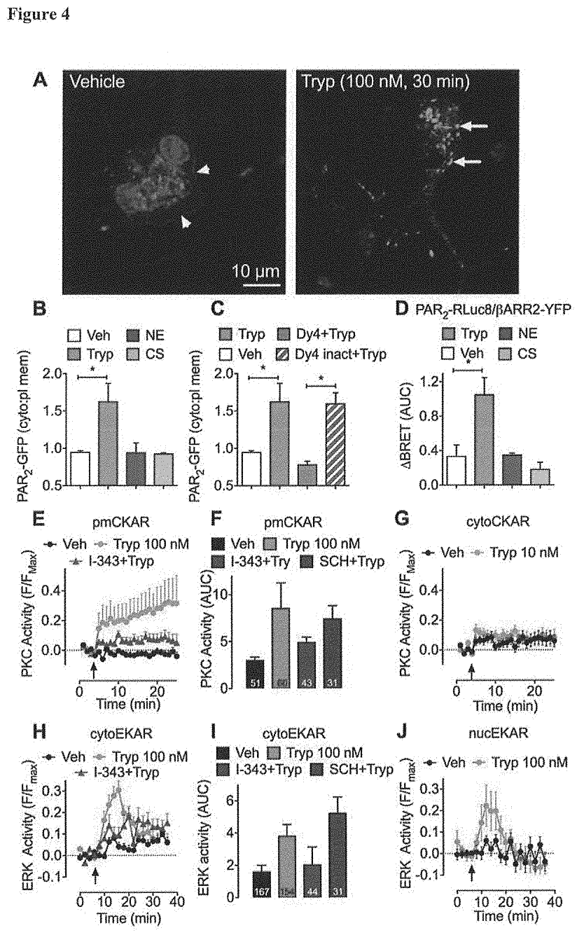

To assess endocytosis of PAR₂ in nociceptors, mouse (m) PAR₂-GFP was transfected in to mouse DRG neurons. In vehicle-treated neurons, mPAR₂-GFP was detected at the plasma membrane and in intracellular compartments that may correspond to stores of PAR₂ in the Golgi apparatus (FIG. 4A) (Jensen D. D., et al. J Biol Chem. 2016, 291(21): 11285-11299). Trypsin, but not NE or CS (100 nM, 30 min), induced mPAR₂-GFP endocytosis (FIG. 4A, B). Dy4, but not Dy4 inact, inhibited trypsin-induced endocytosis of mPAR₂-GFP (FIG. 4C). To determine whether PAR₂ recruits βARRs, which mediate endocytosis, Bioluminescence Resonance Energy Transfer (BRET) sensors were expressed for PAR₂-RLuc8 (donor) and βARR2-YFP (acceptor) in mouse DRG neurons. Trypsin, but not NE or CS, increased PAR₂-RLuc8/βARR2-YFP BRET (FIG. 4D).

To determine whether trypsin causes PAR₂-dependent activation of PKC and ERK, which respectively mediate initial and persistent trypsin-induced hyperexcitability of nociceptors, genetically-encoded Förster Resonance Energy Transfer (FRET) biosensors were expressed in neurons. Biosensors for plasma membrane PKC (pmCKAR), cytosolic PKC (CytoCKAR), cytosolic ERK (CytoEKAR) and nuclear ERK (NucEKAR) (Halls M. L. et al., Methods Mol Biol. 2015, 1335: 131-161) were expressed in DRG neurons from rat, since pilot studies revealed more robust and consistent PAR₂ responses than in mouse neurons. Trypsin (10 or 100 nM) activated PKC at the plasma membrane but not in the cytosol (FIG. 4E-G), and activated ERK in the cytosol and nucleus (FIG. 4H-J). The PAR₂ antagonist I-343 (10 μM) inhibited trypsin-induced activation of PKC and ERK, whereas the PAR₁ antagonist SCH530348 (100 nM) had no effect (FIG. 4F, I). At the end of experiments, neurons were challenged with the positive controls phorbol 12,13-dibutyrate (PDBu) for EKAR biosensors or PDBu plus phosphatase inhibitor mixture-2 for CKAR biosensors, to ensure that the response of the biosensor was not saturated.

The results suggest that trypsin, but not NE or CS, stimulates βARR2 recruitment and dynamin-dependent endocytosis of PAR₂ in nociceptors. Trypsin causes PAR₂-dependent activation of PKC at the plasma membrane and ERK in the cytosol and nucleus.

### Example 31: Mechanisms of PAR₂ Endocytosis and Endosomal Signaling

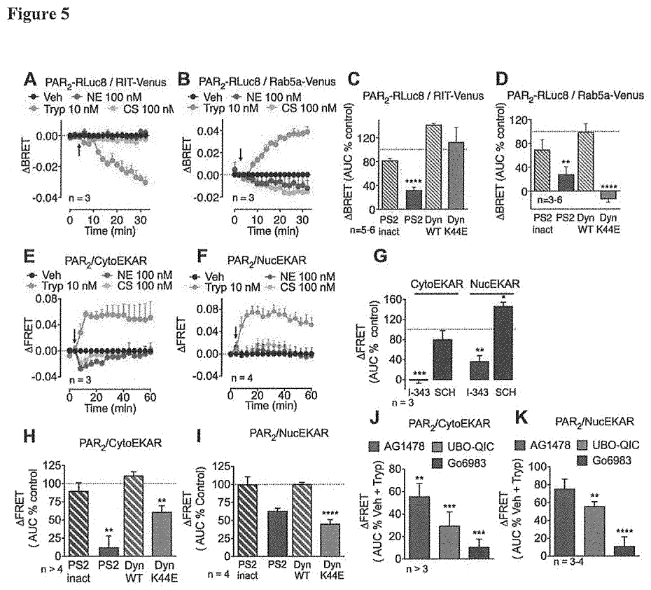

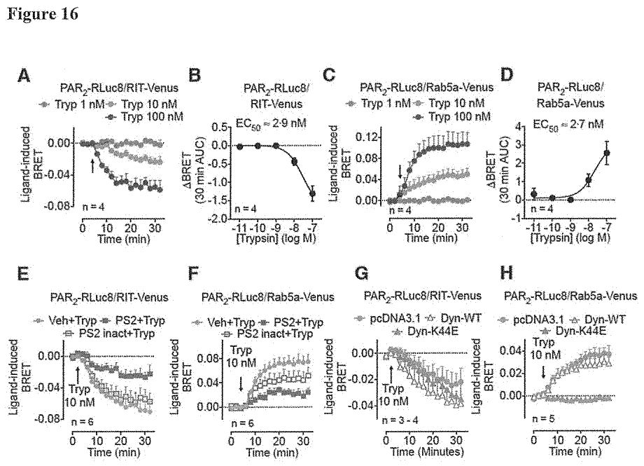

The mechanism of PAR₂ endocytosis and endosomal signaling was examined in HEK293 cells. To quantify the removal of PAR₂ from the plasma membrane and its accumulation in early endosomes, BRET was used to assess the proximity between PAR₂ and proteins that are resident at the plasma membrane (RIT) and early endosomes (Rab5a) (Jensen, D. D. et al., Sci Transl. Med. 2017, 9(392): eaal3447; Yarwood, R. E. et al., Proc. Nat. Acad. Sci. USA 2017, 114(46):12309-12314). This application of BRET takes advantage of nonspecific protein-protein interactions to track movement of membrane proteins through different compartments (Lan, T. H. et al., Traffic 2012, 13(11): 1450-1456). Trypsin induced a decrease in PAR₂-RLuc8/RIT-Venus BRET (EC₅₀ 2.9 nM), and an increase in PAR₂-RLuc8/Rab5a-Venus BRET (EC₅₀ 2.7 nM) (FIGS. 5A, 5B, and 16A-D). Neither NE nor CS (100 nM) affected PAR₂-RLuc8/RIT-Venus or Rab5a-Venus BRET (FIG. 5A, B). PS2, but not PS2 inact, suppressed the trypsin-induced decrease in PAR₂-RLuc8/RIT-Venus BRET and increase in PAR₂-RLuc8/Rab5a-Venus BRET (FIGS. 5C, 5D, 16E, and 16F). Dominant negative dynaminK44E (DynK44E), deficient in GTP binding (Herskovits, J. S. et al., J Cell Biol. 1993, 122(3): 565-578), inhibited the increase in PAR₂-RLuc8/Rab5a-Venus BRET, but did not affect PAR₂-RLuc8/RIT-Venus BRET (FIGS. 5C, 5D, 16G, and 16H). Wild-type dynamin (DynWT) had minimal effects. Since GTP binding is required for scission of budding vesicles from the plasma membrane, DynK44E presumably traps PAR₂ in membrane vesicles, which would impede interaction with Rab5a but not RIT. Thus, trypsin, but not CS or NE, induces clathrin- and dynamin-dependent endocytosis of PAR₂.

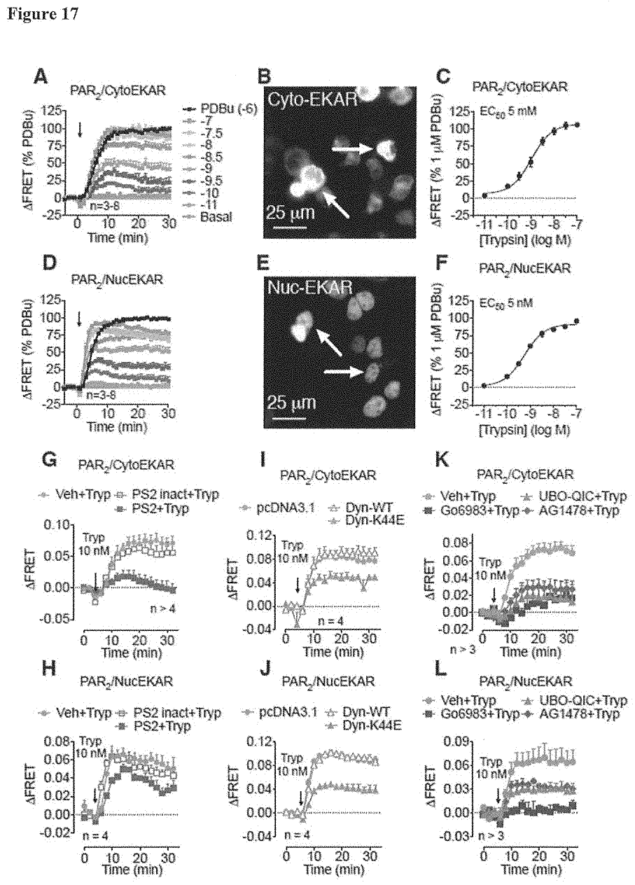

Trypsin-induced ERK signaling mediated by endosomal PAR₂ signaling was investigated in HEK293 cells expressing Flag-PAR₂-HA11 and FRET biosensors for cytosolic and nuclear ERK (CytoEKAR, NucEKAR), plasma membrane and cytosolic PKC (pmCKAR, CytoCKAR), and plasma membrane and cytosolic cAMP (pmEpac, CytoEpac). Trypsin (10 nM), but not NE or CS (100 nM), stimulated a rapid and persistent activation of ERK in the cytosol and nucleus (EC₅₀, 5 nM) (FIG. 5E, 5F, 17A-F). I-343 (10 μM) but not SCH530348 (100 nM) inhibited trypsin activation of cytosolic and nuclear ERK (FIG. 5G). PS2 and DynK44E inhibited trypsin-stimulated activation of cytosolic and nuclear ERK when compared to PS2 inact and DynWT controls (FIG. 5H, 5I, 17G-J). AG1478 (1 μM), an inhibitor of EGF receptor tyrosine kinase (Levitzki, A. & Gazit, A. Science 1995, 267(5205): 1782-1788), UBO-QIC (100 nM), which inhibits Gα_{q} and certain Gβ_{γ} signals (Levitzki, A. et al., Science 1995, 267(5205): 1782-1788), and G66983 (1 μM), which inhibits all isoforms of PKC (Gschwendt, M, et al., FEBS Lett 1996, 392(2): 77-80), suppressed trypsin-stimulated activation of cytosolic ERK (FIGS. 5J and 17K). UBO-QIC and G66983 also inhibited activation of nuclear ERK (FIGS. 5K and 17L). The results suggest that PAR₂ signals from endosomes by Gα_{q}-dependent mechanisms to activate ERK in the cytosol and nucleus.

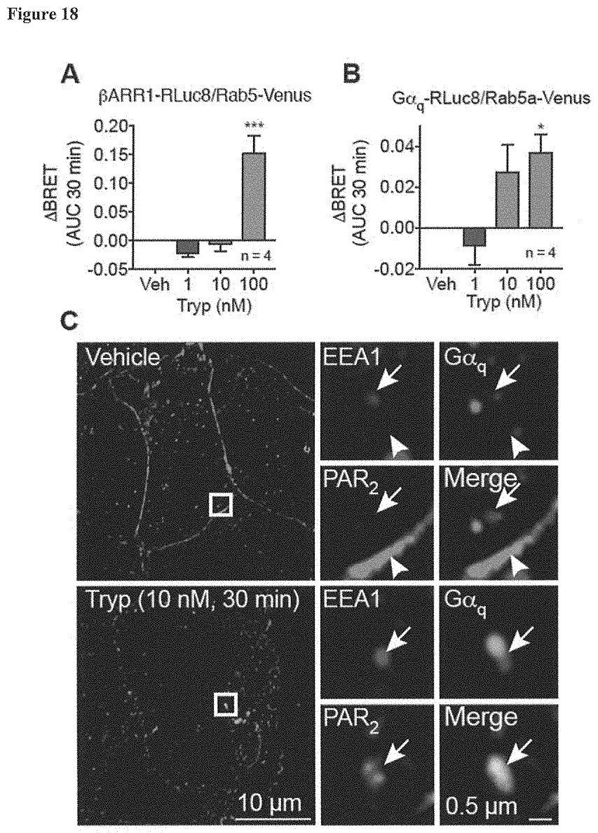

To determine whether trypsin induces translocation of βARR and Gα_{q} to endosomes, we measured BRET between βARR1-RLuc8 or Gα_{q}—RLuc8 and Rab5a-Venus in HEK293 cells. Trypsin (100 nM) stimulated an increase in βARR1-RLuc8/Rab5a-Venus BRET andinGα_{q}-RLuc8/Rab5a-Venus BRET (FIG. 18A, B). Immunofluorescence and structured illumination microscopy were used to localize PAR₂-HA, Gα_{q} and early endosomal antigen-1 (EEA1) in HEK-293 cells. In unstimulated cells, PAR2 was confined to the plasma membrane, although Gα_{q} was detected in early endosomes (FIG. 18C). Trypsin (10 nM, 30 min) induced translocation of PAR₂ to early endosomes containing Gα_{q}. The results support the hypothesis that trypsin causes assembly of a PAR2/βARR/Gα_{q} signalosome in early endosomes.

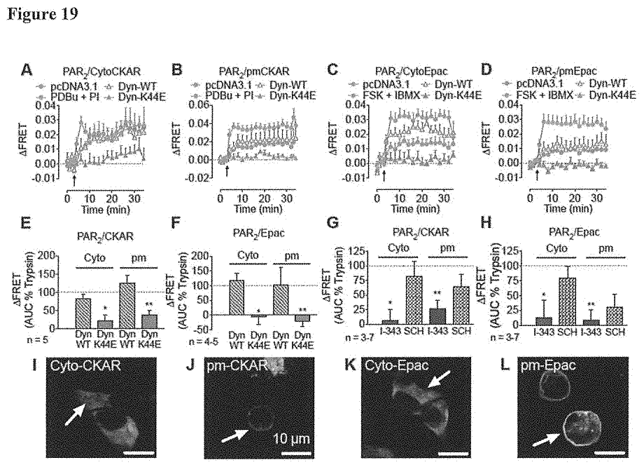

Trypsin (10 nM) caused a rapid and sustained activation of PKC and generation of cAMP at the plasma membrane and in the cytosol of HEK293 cells (FIG. 19A-H). DynK44E strongly inhibited these signals, but DynWT had no effect. I-343, but not SCH530348, inhibited trypsin stimulation of PKC and cAMP, which thus depend on PAR₂ (FIGS. 19G and H). These results suggest that endocytosis is necessary for multiple components of PAR₂ signalling. cAMP signalling at the plasma membrane is usually desensitized by βARR delivery of phosphodiesterases, which degrade cAMP (Perry, S. J. et al., Science 2002, 298(5594): 834-836). The sustained plasma membrane cAMP response to trypsin support the existence of mechanisms that allow persistent PAR₂ signaling, which warrant further investigation. Stimulation of cells with the positive controls PDBu (EKAR), PDBu+phosphatase inhibitor mixture-2 (CKAR), or forskolin+3-isobutyl-1-methylxanthine (Epac) revealed that responses to proteases did not saturate the FRET biosensors (FIGS. 5E, 5F, and 19A-D).

### Example 32: IBS-Induced Hyperexcitability of Nociceptors

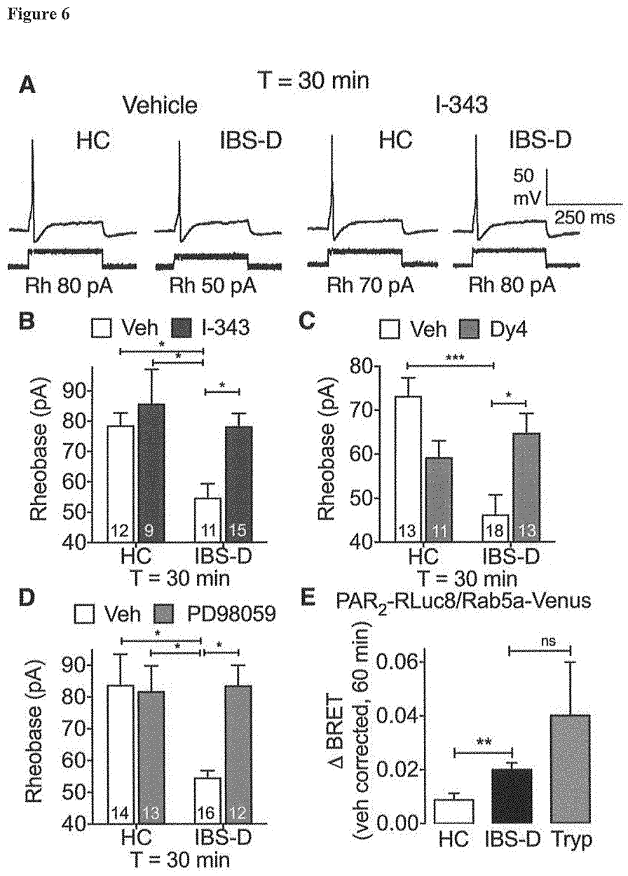

An investigation as whether proteases from mucosal biopsies of IBS patients cause a persistent hyperexcitability of nociceptors was conducted by a mechanism that entails endosomal signaling of PAR₂. Biopsies of colonic mucosa from patients with diarrhea-predominant IBS (IBS-D) or healthy control (HC) subjects were placed in culture medium (24 h, 37° C.). Mouse DRG neurons were then exposed to biopsy supernatants (30 min, 37° C.) and washed. Rheobase was measured 30 min after washing to assess persistent hyperexcitability. Supernatants of biopsies from IBS-D patients caused a persistent decrease in rheobase, consistent with hyperexcitability, when compared to supernatants from HC subjects (rheobase at 30 min: HC, 78.33±4.41 pA, 12 neurons, supernatant from 4 HC; IBS-D, 54.55±4.74 pA, 11 neurons, supernatant from 4 IBS-D; P<0.05; ANOVA, Tukey's multiple comparisons test) (FIG. 6A, B). I-343 (10 μM), Dy4 (dynamin inhibitor, 30 μM) and PD98059 (MEK1 inhibitor, 50 μM) abolished IBS-D-induced hyperexcitability of nociceptors (FIG. 6A-D). Dy4 caused a non-significant decrease in rheobase of neurons exposed to HC supernatant, but I-343 and PD98059 had no effect.

To examine whether proteases in IBS-D supernatants can stimulate endocytosis of PAR₂, BRET was used to assess the proximity between PAR₂-RLuc8 and Rab5a-Venus expressed in HEK293 cells. IBS-D supernatant increased PAR₂-RLuc8/Rab5a-Venus BRET after 60 min when compared to HC supernatant (FIG. 6E). Trypsin (10 nM, positive control) also increased PAR₂-RLuc8/Rab5a-Venus BRET.

These results suggest that proteases that are released from biopsies of colonic mucosa from patients with IBS-D cause long-lasting hyperexcitability of nociceptors by a mechanism that requires dynamin-dependent endocytosis of PAR₂ and PAR₂ ERK signalling from endosomes.

### Example 33: Antagonist Delivery to PAR₂ in Endosomes

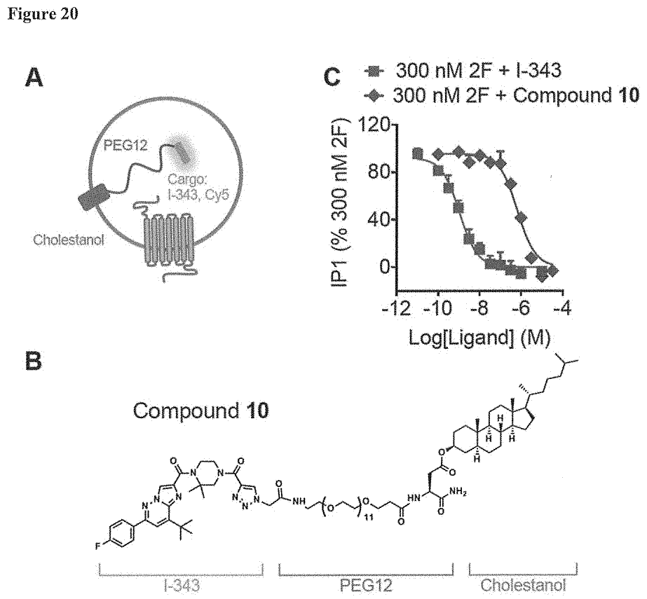

Conjugation to the transmembrane lipid cholestanol facilitates endosomal delivery of antagonists of the neurokinin 1 receptor (NK₁R) and calcitonin receptor-like receptor (CLR), which provide more efficacious and long-lasting anti-nociception (Jensen, D. D. et al., Sci Transl Med, 2017, 9(392):eaal3447; Yarwood R et al., Proc Natl Acad Sci USA 2017, 114(46):12309-12314). To evaluate whether PAR₂ in endosomes is a therapeutic target, tripartite probes were synthesized comprising: cholestanol to anchor probes to membranes or ethyl ester that does not incorporate into membranes; a polyethylene glycol (PEG) 12 linker to facilitate presentation in an aqueous environment; and a cargo of cyanine 5 (Cy5) for localization or PAR₂ antagonist I-343 (FIGS. 20A and B). To determine whether tripartite probes accumulate in endosomes containing PAR₂, mouse DRG neurons expressing mPAR2-GFP were incubated with Cy5-PEG-Cholestanol (Cy5-Chol) or Cy5-PEG-Ethyl ester (Cy5-Ethyl ester) (200 nM, 60 min, 37° C.). Neurons were washed and imaged (37° C.). Cy5-Ethyl ester was not taken up by neurons, whereas Cy5-Chol inserted into the plasma membrane and then accumulated in endosomes of the soma and neurites by 3 h. Trypsin induced endocytosis of PAR₂-GFP into endosomes in close proximity to vesicles containing Cy5-Chol. Video-imaging revealed frequent association of endosomes containing PAR₂-GFP and Cy5-Chol. I-343-PEG-cholestanol (Compound 10, FIG. 20A) antagonized 2F-stimulated IP₁ accumulation in HT-29 cells (pIC₅₀ 6.18±0.07; IC₅₀, 670 nM), albeit with reduced potency compared to the parent compound I-343 (pIC₅₀ 8.96±0.10; IC₅ 1.1 nM) (FIG. 20C).

### Example 34: Antagonism of Endosomal PAR₂ and Hyperexcitability of Nociceptors

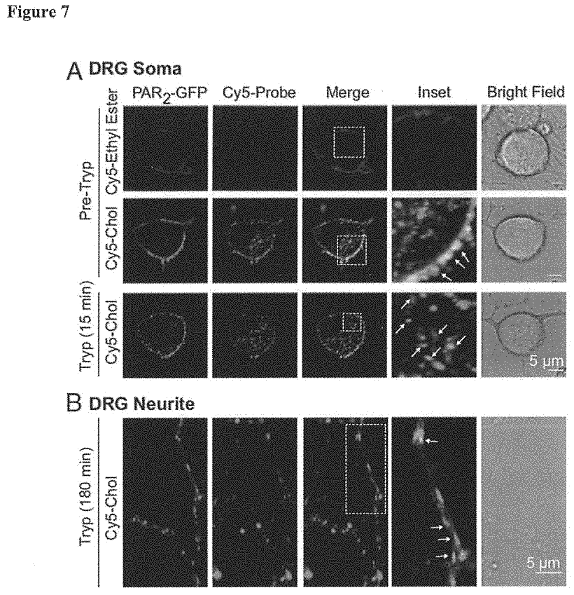

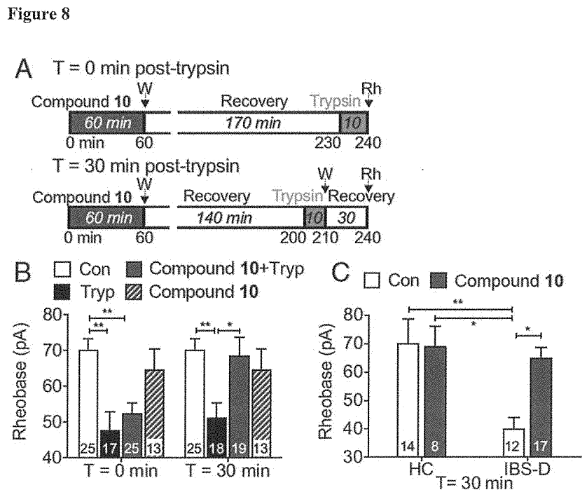

To evaluate the capacity of a compound of the invention to inhibit protease-induced hyperexcitability of nociceptors induced by endosomal PAR₂ signaling, mouse DRG neurons were preincubated with Compound 10 (30 μM) or vehicle (60 min, 37° C.), washed and recovered in antagonist-free medium for 180 min to allow accumulation of antagonist in endosomes (FIG. 8A). Transient incubation with trypsin decreased rheobase of vehicle-treated neurons at 0 and 30 min (FIG. 8B). Compound 10 did not affect the initial excitability at 0 min, but prevented the persistent response at 30 min. Compound 10 had no effect on baseline rheobase. Similarly, transient incubation with IBS-D supernatant decreased rheobase at 30 min compared to HC supernatant (FIG. 8C). Compound 10 completely prevented the persistent actions of IBS-D supernatant on nociceptor excitability (rheobase at 30 min: vehicle IBS-D, 40±3.89 pA, 12 neurons, supernatant from 4 patients; Compound 10 IBS-D, 64.7±3.84 pA, 17 neurons, supernatant from 4 patients; P<0.05) (FIG. 7C). Compound 10 did not affect excitability of neurons treated with HC supernatant.

### Example 35: PAR₂ Endosomal Signaling Mediates Trypsin-Induced Sensitization of Colonic Afferent Neurons

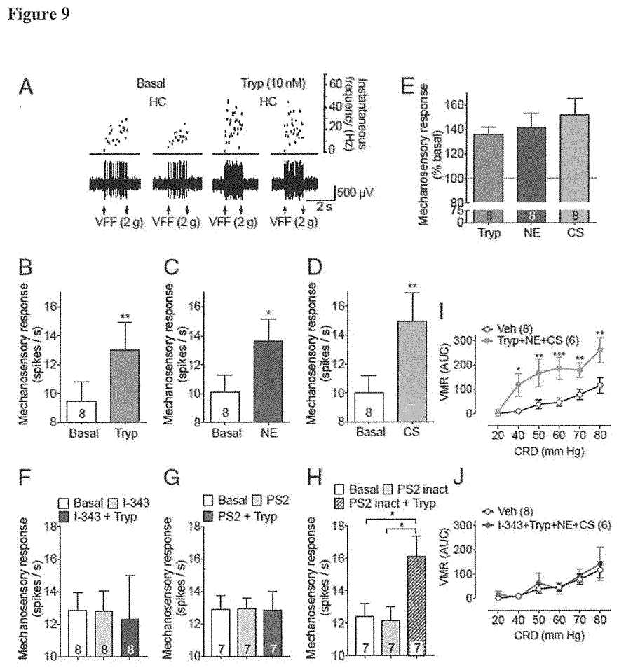

The sensitization of colonic afferent neurons to mechanical stimuli is a leading hypothesis for IBS pain (Azpiroz F. et al., Neurogastroenterol Motil. 2007, 19(1 Suppl): 62-88). To examine whether proteases cleave PAR₂ on the peripheral terminals of colonic nociceptors to induce mechanical hypersensitivity, single unit recordings from afferent neurons innervating the mouse colon were made. Receptive fields were identified by mechanical stimulation of the mucosal surface with von Frey filaments, proteases were applied to the mucosal receptive fields, and mechanical responses were re-evaluated to assess sensitization. Under basal conditions, repeated mechanical stimulation (2 g filament) induced reproducible firing (FIG. 9A). Trypsin (10 nM, 10 min) amplified the frequency of firing to mechanical stimulation by 35.8±5.9%, NE (100 nM, 10 min) by 41.0±11.8%, and CS (100 nM, 10 min) by 52.0±13.2% (FIG. 9B-E).

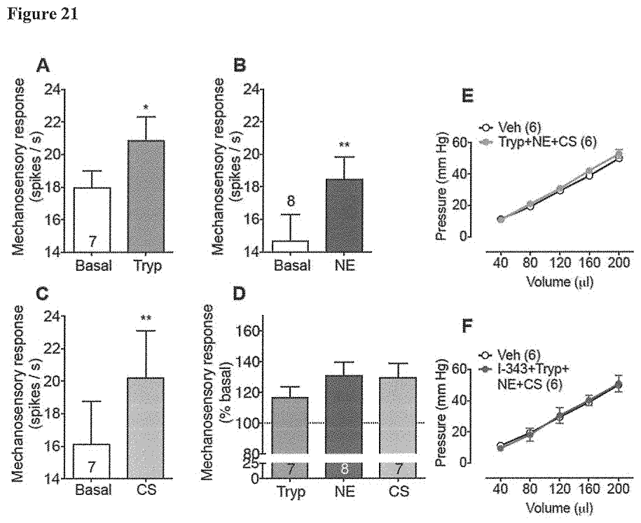

Transient colitis in mice induces hypersensitivity of colonic afferent neurons that persists after inflammation is resolved (Azpiroz F., et al., Neurogastroenterol Motil. 2007, 19(1 Suppl): 62-88). This chronic visceral hypersensitivity (CVH) may mimic post-infectious/inflammatory IBS. To determine whether proteases can further amplify CVH, mice were treated with trinitrobenzene sulphonic acid (TNBS, enema) to induce colitis. At 28 d post-TNBS, when inflammation is undetectable, mechanical stimulation of the colon induced a larger firing rate in CVH mice than in HC mice, consistent with chronic hyperexcitability (FIG. 21A-D). When compared to basal responses, trypsin further amplified responses by 16.4±7.9%, NE by 30.6±9.0% and CS by 29.6±9.2%. Thus, proteases can still amplify the excitability of colonic nociceptors even when they are already sensitized as a result of prior inflammation.

To determine whether endosomal PAR₂ signaling mediates trypsin-induced sensitization of colonic afferent neurons in normal mice, I-343 (10 μM), PS2 or PS2 inact (50 μM) were applied to the receptive fields. I-343 and PS2 did not affect basal mechanical sensitivity, but abolished trypsin-induced sensitization of mechanical responses (FIG. 9F, G). PS2 inact did not affect basal responses or trypsin-induced sensitization (FIG. 9H). The results support the hypothesis that PAR₂ endocytosis is required trypsin-induced sensitization of colonic afferent neurons.

Noxious colorectal distension (CRD) triggers the visceromotor response (VMR), a nociceptive brainstem reflex consisting of contraction of abdominal muscles, which can be monitored by electromyography. This approach allows assessment of visceral sensitivity in awake mice (Castro, J. et al., Br. J. Pharmacol. 2017, 175(12): 2384-2398). To examine protease-induced hy-persensitivity, a protease mixture (10 nM trypsin+100 nM NE+100 nM CS) or vehicle (saline) (100 μL) was instilled into the colon (enema) of healthy mice. After 15 min, the VMR was measured in response to graded CRD (20-80 mm Hg) with a barostat balloon. In vehicle-treated mice, CRD induced a graded VMR (FIG. 9I). The protease mixture amplified VMR at all pressures from 40 to 80 mm Hg. Administration of I-343 (30 mg/kg) into the colon (100 μL enema) 30 min before the protease mixture, abolished the response (FIG. 9J). Because alterations in the compliance of the colon can alter VMR to CRD, the pressure/volume relationship was measured at all distending pressures. Compliance of the colon was unaffected by the protease mixture or I-343 (FIGS. 21E and F). The results support the hypothesis that PAR₂ endocytosis is required for trypsin-induced sensitization of colonic afferent neurons and colonic nociception.

### Materials and Methods

### Human Subjects.

The Queen's University Human Ethics Committee approved human studies. All subjects gave informed consent. Endoscopic biopsies were obtained from the descending colon of 13 adult IBS-D patients (12 female) diagnosed using ROME III criteria for diarrhea predominant IBS and of 12 health controls. All IBS patients had symptoms greater than 1 year and most were greater than 5 years. Celiac disease was excluded by blood test and patients over 40 years with daily diarrhea were biopsied at the time of colonoscopy to exclude microscopic colitis. None of the patients had a history suggestive of post-infectious IBS. Control biopsies were obtained from patients undergoing colon screening who did not have gastrointestinal symptoms. Biopsies (8 samples per patient) were incubated in 250 μl of RPMI medium containing 10% fetal calf serum, penicillin/streptomycin and gentamicin/amphotericin B (95% O₂/5% C₂, 24 h, 37° C.). Supernatants were stored at −80° C. Supernatants from 4-6 patients were pooled and studied in individual experiments.

### Animal Subjects.

Institutional Animal Care and Use Committees of Queen's, Monash, Flinders and New York Universities and the South Australian Health and Medical Research Institute approved studies of mice and rats. Mice (C57BL/6, males, 6-15 weeks) and rats (Sprague-Dawley, males, 8-12 weeks) were studied. Animals were maintained in a temperature-controlled environment with a 12 h light/dark cycle and free access to food and water. Animals were killed by CO₂ inhalation or anesthetic overdose and thoracotomy. Animals were randomized for treatments and no animals were excluded from studies.

Par₂-Na_{V}1.8 Mice.

F2rl1 conditional knock-out C57BL/6 mice were generated by genOway (Lyon, France). The last exon of F2rl1, encoding for the transmembrane, extracellular and cytoplasmic domains of F2RL1, was flanked by loxP sites and a neomycin cassette in intron 1. The neomycin cassette was excised by breeding these mice with a C57BL/6 Flp-expressing mouse line. To delete Par₂ in peripheral neurons, F2rl1 conditional knock-out mice were bred with mice expressing Cre recombinase under the control of the Scn10a gene promoter (B6.129-Scn10a^{tm2(cre)Jnw/H}). Deletion of PAR₂ in Na_{V}1.8 nociceptors was evaluated by immunofluorescence. DRGs from wild-type and Par₂-Na_{V}1.8 mice were fixed in 10% formalin for 3 h, transferred to 70% alcohol, and embedded in paraffin. Sections (5 μm) were deparaffinized, rehydrated, microwaved in sodium citrate buffer, washed, and then blocked in SuperBlock™ (ThermoFisher Scientific) for one hour at room temperature. Sections were incubated with mouse antibody to PAR₂ conjugated to Alexa-488 (Santa Cruz Biotechnology, SC-13504, 1:200, 4° C., overnight), and with guinea pig antibody to Na_{V}1.8 (Alomone Labs, AGP-029, 1:200, 4° C., overnight), followed by goat anti-guinea pig secondary antibody conjugated to Alexa Fluor-594 (Life Technologies, A11076, 1:500, room temperature, 1 hour). Sections were imaged with a Nikon Eclipse Ti microscope using 10× magnification; images were captured with a Photometrics CoolSNAP camera.

### Somatic Nociception and Inflammation.

Mice were acclimatized to the experimental apparatus, room and investigator for 1-2 h on 2 successive days before studies. Investigators were blinded to the test agents. Mice were sedated (5% isoflurane) for intraplantar injections. Dy4a, Dy4 inact, PS2, PS2 inact (all 50 μM) or vehicle (0.2% DMSO in 0.9% NaCl) (10 μl) was injected into the left hindpaw. After 30 min, trypsin (10 or 80 nM), CS (2.5 or 5 μM) or NE (1.2 or 3.9 μM) (all 10 μl) was injected into the same hindpaw. Mechanical nociceptive responses were evaluated by examining paw withdrawal to stimulation of the plantar surface of the hind-paw with calibrated von Frey filaments. von Frey scores were measured in triplicate to establish a baseline for each animal on the day before experiments, and were then measured for up to 4 h after protease administration. To assess edema, paw thickness was measured at the site of injection between the plantar and the dorsal surfaces of the paw using digital calipers. For evaluation of neutrophil infiltration, paws were collected at 4 h after intraplantar injection of trypsin (10 μl, 80 nM) or vehicle, fixed in 10% neutral buffered formalin for 48-72 h, bisected, and fixed in formalin for an additional 12 h. Tissue was decalcified in 10% 0.5 M EDTA for 6 days, washed in water, transferred to 70% ethanol for 24 h, and embedded in paraffin. Sections (5 μm) were incubated with neutrophil antibody Ly6G/6C clone NIMP-R14 (Abcam #ab2557, Lot #GR135037-1, AB_303154, 1:800, room temperature, 12 h). Sections were processed for chromogenic immunohistochemistry on a Ventana Medical Systems Discovery XT platform with online deparaffinization using Ventana's reagents. Ly6G/Ly6c was enzymatically treated with protease-3 (Ventana Medical Systems) for 8 min. Ly6G/Ly6c was detected with goat anti-rat horseradish peroxidase conjugated multimer incubated for 16 min.

### Dissociation of DRG Neurons for Electrophysiological Studies.

DRG innervating the colon (T9-T13) were collected from C57BL/6 mice. Ganglia were digested by incubation in collagenase IV (1 mg/ml, Worthington) and dispase (4 mg/ml, Roche) (10 min, 37° C.). DRG were triturated with a fire-polished Pasteur pipette, and further digested (5 min, 37° C.). Neurons were washed, plated onto laminin- (0.017 mg/ml) and poly-D-lysine- (2 mg/ml) coated glass coverslips, and were maintained in F12 medium containing 10% fetal calf serum, penicillin and streptomycin (95% air, 5% CO₂, 16 h, 37° C.) until retrieval for electrophysiological studies.

### Patch Clamp Recording.

Small-diameter (<30 pF capacitance) neurons were studied because they display characteristics of nociceptors (Valdez-Morales E. E. et al., Am J Gastroenterol 2013, 108(10): 1634-1643). Changes in excitability were quantified by measuring rheobase. Whole-cell perforated patch-clamp recordings were made using Amphotericin B (240 μg/ml, Sigma Aldrich) in current clamp mode at room temperature. The recording chamber was perfused with external solution at 2 ml/min. Recordings were made using Multiclamp 700B or Axopatch 200B amplifiers, digitized by Digidata 1440A or 1322A, and processed using pClamp 10.1 software (Molecular Devices). Solutions had the composition (mM): pipette—K-gluconate 110, KC130, HEPES 10, MgCl₂ 1, CaCl₂ 2; pH 7.25 with 1 M KOH; external—NaCl 140, KCl 5 HEPES 10, glucose 10, MgCl₂ 1, CaCl₂ 2; pH to 7.3-7.4 with 3 M NaOH. Neurons were preincubated with supernatants of colonic mucosal biopsies from HC or IBS-D subjects (200 μl supernatant were combined with 500 μl of F12 medium, filtered) for 30 min. Neurons were also preincubated with trypsin (50 nM, 10 min), NE (390 nM, 30 min), CS (500 nM, 60 min), or vehicle (37° C.), and washed. Rheobase was measured at T 0 or T 30 min after washing. To investigate mechanisms of protease-evoked effects, neurons were incubated with I-343 (100 nM, 300 nM, 10 μM, 30 min preincubation), SCH79797 (1 μM, 10 min), Dy4 (30 μM, 30 min), PS2 (15 μM, 30 min), PD98059 (50 μM, 30 min), GF109203X (10 μM, 30 min), or vehicle (preincubation and inclusion throughout). In experiments using the tripartite antagonist, neurons were preincubated with Compound 10 (30 μM, 60 min, 37° C.) or vehicle and washed. They were recovered in F12 medium at 37° C. for variable times, challenged with HC or IBS-D supernatant or trypsin (50 nM, 10 min), and washed. Rheobase was measured 0 or 30 min after washing. In all experiments, the mean rheobase was calculated for neurons exposed to supernatants, proteases or vehicle.

### Colonic Afferent Recordings.

The colon and rectum (5-6 cm) was removed from C57BL/6 mice. Afferent recordings were made from splanchnic nerves as described (Hughes, P. A. et al., Gut 2009, 58(10): 1333-134; Brierley, S. M. et al., Gastroenterology 2004, 127(1): 166-178). Briefly, the intestine was opened and pinned flat, mucosal side up, in an organ bath. Tissue was superfused with a modified Krebs solution (mM: 117.9 NaCl, 4.7 KCl, NaHCO₃, 1.3 NaH₂PO₄, 1.2 MgSO₄ (H₂O)₇, 2.5 CaCl₂, 11.1 D-glucose; 95% O₂, 5% CO₂, 34° C.), containing the L-type calcium channel antagonist nifedipine (1 μM) to suppress smooth muscle activity, and the cyclooxygenase inhibitor indomethacin (3 μM) to suppress inhibitory actions of prostaglandins. The splanchnic nerve was extended into a paraffin-filled recording compartment, in which finely dissected strands were laid onto a platinum electrode for single-unit extracellular recordings of action potentials generated by mechanical stimulation of receptive fields in the colon. Receptive fields were identified by mechanical stimulation of the mucosal surface with a brush of sufficient stiffness to activate all types of mechanosensitive afferents. Once identified, receptive fields were tested with three distinct mechanical stimuli to enable classification: static probing with calibrated von Frey filaments (2 g force; 3 times for 3 sec), mucosal stroking with von Frey filaments (10 mg force; 10 times), or circular stretch (5 g; 1 min). Colonic nociceptors displayed high-mechanical activation thresholds and responded to noxious distension (40 mmHg), circular stretch (≥7 g) or 2 g filament probing, but not to fine mucosal stroking (10 mg filament). These neurons express an array of channels and receptors involved in pain, become mechanically hypersensitive in models of chronic visceral pain, and have a nociceptor phenotype. They are therefore referred to as “colonic nociceptors”. Once baseline colonic nociceptor responses to mechanical stimuli (2 g filament) had been established, mechanosensitivity was re-tested after 10 min application of trypsin (10 nM), NE (100 nM) or CS (100 nM). Proteases were applied to a metal cylinder placed over the receptive mucosal field of interest. This route of administration has been shown to activate colonic afferents (Hughes, P. A. et al., Gut 2009, 58(10): 1333-134). Action potentials were analyzed using the Spike 2 wavemark function and discriminated as single units on the basis of distinguishable waveform, amplitude and duration.

### Colonic Visceral Hypersensitivity (CVH).

CVH was induced by intracolonic administration of trinitrobenzene sulphonic acid (TNBS) as described (Hughes, P. A., et al., Gut 2009, 58(10): 1333-134; Brierley, S. M. et al., Gastroenterology 2004, 127(1): 166-178). Briefly, 12 week old mice were fasted overnight with access to 5% glucose solution. TNBS (100 μl, containing 4 mg TNBS in 30% EtOH) was administered to sedated mice (5% isoflurane) through a polyethylene catheter inserted 3 cm past the anus. Mice were then allowed to recover for 28 days. At this time, mice display colonic mechanical hypersensitivity, allodynia and hyperalgesia. They are therefore termed CVH mice.

### Visceromotor Responses (VMR) to Colorectal Distension (CRD).

Electromyography (EMG) of abdominal muscles was used to monitor VMR to CRD (Eichel, K. et al., Nat. Cell Biol. 2016, 18(3): 303-310). Electrodes were implanted into the right abdominal muscle of mice under isoflurane anesthesia. Mice were recovered for at least three days before assessment of VMR. On the day of VMR assessment, mice were sedated with isoflurane, and vehicle (saline) or protease cocktail (10 nM trypsin, 100 nM NE, 100 nM CS) (100 μl) was administered into the colon via enema. In one group of mice, I-343 (30 mg/kg, 100 μl) was administered into the colon 30 min before the protease cocktail. A lubricated balloon (2.5 cm) was introduced into the colorectum to 0.25 cm past the anus. The balloon catheter was secured to the base of the tail and connected to a barostat (Isobar 3, G&J Electronics) for graded and pressure-controlled balloon distension. Mice were allowed to recover from anesthesia for 15 min before the CRD sequence. Distensions were applied at 20, 40, 50, 60, 70 and 80 mm Hg (20 s duration) at 4-min intervals; the final distension was 30 min after administration of protease or vehicle. The EMG signal was recorded (NL100AK headstage), amplified (NL104), filtered (NL 125/126, Neurolog, Digitimer Ltd, bandpass 50-5000 Hz), and digitized (CED 1401, Cambridge Electronic Design) for off-line analysis using Spike2 (Cambridge Electronic Design). The analog EMG signal was rectified and integrated. To quantify the magnitude of the VMR at each distension pressure, the area under the curve (AUC) during the distension (20 s) was corrected for the baseline activity (AUC pre-distension, 20 s). Colonic compliance was assessed by applying graded volumes (40-200 μl, 20 s duration) to the balloon in awake mice, while recording the corresponding colorectal pressure, as described (Eichel, K. et al., Nat. Cell Biol. 2016, 18(3): 303-310; Irannejad, R. et al., Nature 2013, 495(7442): 534-538).

Dissociation of DRG neurons for signaling and trafficking studies.

DRG were collected from C57BL/6 mice and Sprague-Dawley rats (all levels). DRG were incubated with collagenase IV (2 mg/ml) and dispase II (1 mg/ml) for 30 min (mice) and 45 min (rats) at 37° C. DRG were dispersed by trituration with a fire-polished Pasteur pipette. Dissociated neurons were transfected with mPAR₂-GFP (1 μg), the FRET biosensors CytoEKAR, NucEKAR, pmCKAR or CytoCKAR (all 1 g), or with the BRET biosensors PAR₂-RLuc8 (125 ng) and βARR2-FYP (475 ng) using the Lonza 4D-Nucleofector X unit according to the manufacturer's instructions. Neurons were plated on laminin- (0.004 mg/ml) and poly-L-Lysine- (0.1 mg/ml) coated glass coverslips for confocal microscopy, on ViewPlate-96 plates (PerkinElmer) for FRET assays, or on CulturPlates (PerkinElmer) for BRET assays. Neurons were maintained in Dulbecco's modified Eagle medium (DMEM) containing 10% fetal bovine serum (FBS), antibiotic-antimitotic, and N1 supplement for 48 h before study.

### PAR₂ Trafficking in DRG Neurons.

Mouse DRG neurons expressing mPAR₂-GFP were incubated with trypsin (10 nM), CS (100 nM), NE (100 nM) or vehicle (30 min, 37° C.), and were fixed (4% paraformaldehyde, 20 min, 4C). NeuN was detected by indirect immunofluorescence as described (Jensen, D. D. et al., Sci Transl Med. 2017, 9(392), eaal3447). Neurons were observed using a Leica SP8 confocal microscope with a HCX PL APO 63× (NA 1.40) oil objective. PAR₂ internalization in NeuN-positive neurons was quantified using ImageJ software. The border of the cytoplasm in the neuronal soma was defined by NeuN fluorescence. mPAR₂-GFP fluorescence within 0.5 μm of the border was defined as plasma membrane-associated receptor. The ratio of plasma membrane to cytosolic mPAR₂-GFP was determined.

### FRET Assays in DRG Neurons.

Rat DRG neurons expressing FRET biosensors were serum-restricted (0.5% FBS overnight), and equilibrated in HBSS-HEPES (10 mM HEPES, pH 7.4, 30 min, 37° C.). FRET was analyzed using an Operetta CLS High-Content Imaging System (PerkinElmer) or an INCell Analyzer 2000 G E Healthcare Life Sciences). For CFP/YFP emission ratio analysis, cells were sequentially excited using a CFP filter (410-430 nm) with emission measured using YFP (520-560 nm) and CFP (460-500 nm) filters. Cells were imaged at 1 or 2 min intervals. Baseline was measured, neurons were challenged with trypsin (10 or 100 nM) or vehicle, and responses recorded for a further 30 min. Neurons were then challenged with phorbol 12,13-dibutyrate (PDBu, 200 nM, 10 min) for EKAR biosensors or PDBu (200 nM) and phosphatase inhibitor cocktail-2 (SigmaAldrich, 10 min) for CKAR biosensors. Data were analyzed using Harmony 4.1 or Image J 1.51 software. Images taken were aligned, cells were selected based on diameter, and fluorescence intensity was calculated for both FRET and CFP channels. Background intensity was subtracted and the FRET ratio was determined as the change in the FRET/donor (EKAR) or donor/FRET (CKAR) emission ratio relative to the baseline for each cell (F/Fo). Cells with >5% changes in F/F0 after PDBu stimulation were selected for analysis. Neurons were incubated with I-343 (10 μM), SCH530348 (100 nM) or vehicle (30 min, 37° C. preincubation and inclusion throughout).

### BRET Assays in DRG Neurons.

Mouse DRG neurons were equilibrated in HBSS-HEPES (30 min, 37° C.), and incubated with the Renilla luciferase substrate coelenterazine h (NanoLight Technologies) (5 μM, 5 min). BRET ratios were measured at 475±30 nm and 535±30 nm using a CLARIOstar Monochronometer Microplate Reader (BMG LabTech) before and after challenge with trypsin (10 nM), NE (100 nM) or CS (100 nM). Data are presented as a BRET ratio, calculated as the ratio of YFP to RLuc8 signals, and normalized to the baseline average. Data are plotted as area under the curve for the 25 min assay.

### Ca²⁺ Assays in DRG Neurons.

[Ca²⁺]ᵢ was measured in DRG neurons from WT and Par₂-Na_{V}1.8 mice, as described (Tsvetanova, N. G., et al., Nat. Chem. Biol. 2014, 10(12): 1061-1065). Neurons were loaded with Fura-2AM (1 μM) in Ca²⁺- and Mg²⁺-containing DMEM (45 min, room temperature). Fluorescence of individual neurons was measured at 340 nm and 380 nm excitation and 530 nm using a Nikon Eclipse Ti microscope with 20× magnification and a Photometrics CoolSNAP camera. Data were analyzed using Nikon Ti Element Software. Cultures were first challenged with KCl (65 mM), to identify responsive neurons, and were then exposed to trypsin (100 nM). Cells≤25 μm diameter were selected for analysis. For determination of the activation threshold, the magnitude of the 340/380 ratio after exposure to trypsin was compared to the baseline ratio. Neurons were considered responsive to trypsin if the 340/380 ratio was >0.1 from baseline.

### Uptake of Tripartite Probes in DRG Neurons.

Mouse DRG neurons expressing mPAR₂-GFP were incubated with Cy5-Ethyl ester (control) or Cy5-Chol (200 nM, 60 min, 37° C.) and then washed in HBSS-HEPES. Neurons were transferred to a heated chamber (37° C.) in HBSS-HEPES and were observed by confocal microscopy before or after treatment with trypsin (100 nM, 15 min). Images were obtained using a Leica TCS SP8 Laser-scanning confocal microscope with a HCX PL APO 63× (NA 1.40) oil objective. Image acquisition settings were consistent for Cy5-Chol and Cy5-ethyl ester fluorescence detection.

### Cell Lines, Transfection.

HEK293 cells were cultured in DMEM supplemented with 10% (v/v) FBS (5% CO₂, 37° C.). When necessary serum restriction was achieved by replacing culture medium with DMEM containing 0.5% FBS overnight. Cells were transiently transfected using polyethylenimine (PEI) (1:6 DNA:PEI).

### FRET Assays in HEK293 Cells.

HEK293 cells were transiently transfected in 10 cm dishes (˜50% confluency) with Flag-PAR2-HA (2.5 μg) and FRET biosensors CytoEKAR or NucEKAR (2.5 μg) (Jensen, D. D. et al., Sci Transl Med. 2017, 9(392), eaal3447; Thomsen, A. R. B., et al., Cell 2016, 166(4): 907-919). In experiments examining the role of dynamin, cells were transfected with FLAG-PAR₂-HA (1.25 μg), FRET biosensor (1.25 μg) and either DynWT-HA, DynK44E-HA or pcDNA3.1 (2.5 μg). At 24 h after transfection, cells were seeded on ViewPlate-96 well plates (PerkinElmer). FRET was assessed 72 h post-transfection, following overnight serum restriction. Cells were equilibrated in HBSS-HEPES (30 min, 37° C.). FRET was measured using a PHERAstar FSX Microplate Reader (BMG LabTech). For CFP/YFP emission ratio analysis, cells were sequentially excited using a CFP filter (425/10 nm) with emission measured using YFP (550/50 nm) and CFP (490/20 nm) filters. FRET was measured before and after stimulation with trypsin (10 nM), NE (100 nM), CS (100 nM), phorbol 12,13-dibutyrate, PDBu (positive control, 1 μM), or vehicle. FRET ratios (donor/acceptor intensity for EKAR, or acceptor/donor intensity for CKAR and Epac) were calculated and corrected to baseline and vehicle treatments to determine ligand-induced FRET (ΔFRET). Treatment effects were determined by comparison of area under the curve values. Signalling inhibitors were dissolved in HBSS-HEPES. PS2 and PS2 inact were dissolved in HBSS-HEPES+1% DMSO. Cells were incubated with UBO-QIC (100 nM), AG1478 (1 μM), G66983 (1 μM), PS2 or PS2 inact (30 μM) or vehicle (30 min preincubation, inclusion throughout).

### BRET Assays in HEK293 Cells.

HEK293 cells were transiently transfected in 10 cm dishes (˜50% confluency) with: PAR₂-RLuc8 (1 μg) and either RIT-Venus or Rab5a-Venus (both 4 μg); Flag-PAR₂-HA (1 μg) and βARR1-RLuc8 (1 μg) plus Rab5a-Venus (4 μg); or Flag-PAR₂-HA (1 μg) and Gα_{q}-RLuc8 (0.5 μg), Gb (1 μg), Gg (1 μg) and Rab5a-Venus (4 μg). To examine the role of dynamin, cells were transfected with PAR₂-RLuc8 (0.5 μg), RIT-Venus or Rab5a-Venus (2 μg), and DynWT-HA, DynK44E-HA or pcDNA3.1 (2.5 μg). At 24 h after transfection, cells were seeded on CulturPlates (PerkinElmer). The following day, cells were equilibrated in HBSS-HEPES and incubated with coelenterazine h (NanoLight Technologies) (5 μM, 5 min). RLuc8 and YFP intensities were measured at 475±30 nm and 535±30 nm, respectively, using a LUMIstar Omega Microplate Reader (BMG LabTech) before and after challenge with proteases, biopsy supernatants or vehicle. Data are presented as a BRET ratio, calculated as the ratio of YFP to RLuc8 signals, and normalized to the baseline average, followed by vehicle subtraction. Treatment effects were determined by comparison of area under the curve values.

### Immunofluorescence and Structured Illumination Microscopy.

HEK293 cells transiently expressing Flag-PAR₂-HA were seeded on poly-D-lysine-coated high tolerance cover-glass and incubated overnight. Cells were incubated with trypsin (10 nM) or vehicle in DMEM for 30 min at 37° C. Cells were fixed in 4% paraformaldehyde on ice for 20 min and washed in PBS. Cells were blocked for 1 h at room temperature in PBS+0.3% saponin+3% NHS. Cells were incubated with primary antibodies to HA (rat anti-HA, 1:1,000, Roche), EEA-1 (rabbit anti-EEA-1 1:100, Abcam), Gα_{q} (mouse anti-GNAQ 1:100, Millipore) in PBS+0.3% saponin+1% NHS overnight at 4° C. Cells were washed in PBS and incubated with secondary antibodies (goat anti-Rat Alexa568, donkey anti-rabbit Alexa488, goat anti-mouse Dylight405, 1:1,000, Invitrogen) for 1 h at room temperature. Cells were washed with PBS and mounted on glass slides with prolong Diamond mounting medium (ThermoFisher). Cells were observed by super-resolution structured illumination microscopy (SIM) using a Nikon N-SIM Eclipse TiE inverted microscope with an SR Apo-TIRF100×/1.49 objective. Images were acquired in 3D-SIM mode using 405, 488, and 561 nm lasers and filter sets for standard blue, green, and red emission on an Andor iXon 3 EMCCD camera. Z-stacks were collected with a 125 nm z interval. NIS-Elements AR Software was used to reconstruct SIM images.

cDNAs.

BRET sensors PAR₂-RLuc8, KRas-Venus, Rab5a-Venus and βARR2-YFP have been described (Jensen, D. D. et al., Sci Transl Med. 2017, 9(392): eaal3447; Yarwood, R. et al., Proc Natl Acad Sci USA 2017, 114(46): 12309-12314). FRET sensors CytoEKAR, NucEKAR, CytoCKAR and pmCKAR were from Addgene (plasmids 18680, 18681, 14870, 14862, respectively).

### IP₁ Accumulation Assay.

KNRK-hPAR₂, KNRK, HEK293, or HT-29 cells were seeded at a density of 50×103 cells/well onto clear 96-well plates (PerkinElmer). After 24 h of culture, medium was replaced with IP₁ stimulation buffer (10 mM HEPES, 1 mM CaCl₂, 0.5 mM MgCl₂, 4.2 mM KCl, 146 mM NaCl, 5.5 mM glucose, 50 mM LiCl; 37° C., 5% CO₂.). Cells were pre-incubated with the antagonist or vehicle for 30 min prior to the addition of agonist. Cells were then further incubated for 40 min. Stimulation buffer was

1. A compound of Formula (I): 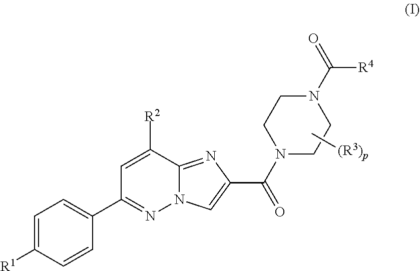
or pharmaceutically acceptable salt thereof wherein:
R¹ is H, C₁-C₆ alkyl or halo;
R² is C₁-C₆ alkyl, C₃-C₆ cycloalkyl or C₁-C₆ aryl, each optionally substituted with 1 to 3 halogens;
R³ is oxo or C₁-C₆ alkyl;
p is an integer from 0 to 3;
R⁴ is —C₁-C₆ alkylS(O)₂OH, -1,2,3-triazol-1-acetic acid, —NHR⁷, -bicycle[2.2.2]octaneC(O)OR⁶, —C₄-C₈ cycloalkyl-R⁵, a 4-6 membered heterocyclic or heteroaryl group substituted with —C₁-C₆ alkyl-R⁵, or —(CH₂)₂C(O)NHC₂-C₁₀ alkyl, wherein the C₂-C₁₀ alkyl is substituted with 2 to 10 —NH₂ or —OH;
R⁵ is —C(O)NHR⁷ or —NHC(O)R⁷;
R⁶ is H or R⁷
R⁷ is —R⁸, —C₁-C₂₀ alkyl, —C₁-C₂₀ alkylC(O)NH₂ or —C₁-C₂₀ alkylC(O)NR⁸, wherein the —C₁-C₂₀ alkyl, —C₁-C₂₀ alkylC(O)NH₂ and —C₁-C₂₀ alkylC(O)NR⁸ are optionally substituted with 2 to 10 —NH₂ or —OH, and wherein one or more of the carbon atoms in the alkyl group are optionally replaced with nitrogen or oxygen;
R⁸ is represented by the formula: 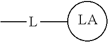
wherein
L is a linker moiety of 1 nm to 50 nm in length; and
LA is a lipid anchor that promotes insertion of the compound into a plasma membrane.

2. The compound according to claim 1 or pharmaceutically acceptable salt thereof, wherein R¹ is halo and R² is C₁-C₆ alkyl.

3. The compound according to claim 2 or pharmaceutically acceptable salt thereof, wherein R¹ is fluoro and R² is a t-butyl group.

4. The compound according to any one of claims 1 to 3 or pharmaceutically acceptable salt thereof, wherein R³ is C₁-C₆ alkyl and p is 2.

5. The compound according to claim 4 or pharmaceutically acceptable salt thereof, wherein R³ is methyl and p is 2.

6. The compound according to any one of claims 1 to 5 or pharmaceutically acceptable salt thereof, wherein the lipid anchor (LA) partitions into lipid membranes that are insoluble in non-ionic detergent at 4° C.

7. The compound according to any one of claims 1 to 6 or pharmaceutically acceptable salt thereof, wherein the lipid anchor (LA) that promotes insertion of the compound into a plasma membrane is represented by formulae (IIa), (IIIa), or (IVa): 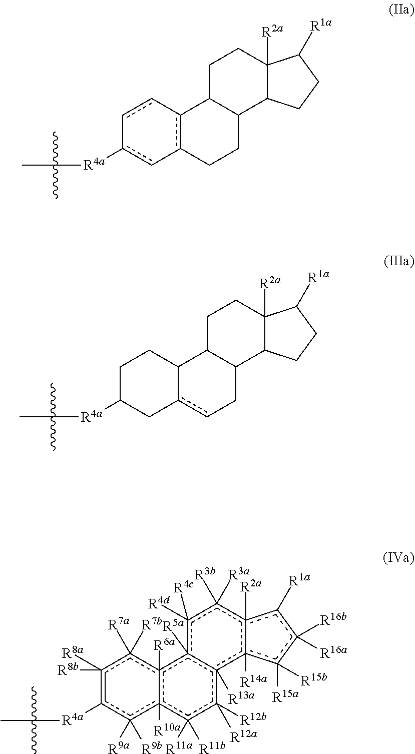
wherein
R^{1a} is an optionally substituted C₁₋₁₂ alkyl, alkenyl, alkynyl or alkoxy group;
R^{2a} and R^{3a}, R^{3b}, R^{4b}, R^{4c}, R^{5a}, R^{6a}, R^{7a}, R^{7b} R^{8a}, R^{8b}, R^{9a}, R^{9b}, R^{10a}, R^{11a}, R^{11b}, R^{12a}, R^{12b} R^{13a}, R^{14a}, R^{15a}, R^{15b}, R^{16a} and R^{16b} are independently H, C₁₋₃ alkyl, hydroxyl, C₁₋₃ alkoxy or amino; or
optionally, R^{3a}, R^{3b} and/or R^{4b}, R^{4c}, and/or R^{7a}, R^{7b} and/or R^{8a}, R^{8b} and/or R^{11a}, R^{11b} and/or R^{12a}, R^{12b} and/or R^{15a}, R^{15b} and R^{16a}, R^{16b} are taken together to give ═O (double bond to oxygen);
R^{4a} is C, O, NH or S; and [symbol]
represents a single or double bond.

8. The compound according to any one of claims 1 to 7 or pharmaceutically acceptable salt thereof, wherein L is a linker moiety of 1 nm to 50 nm in length, wherein L is represented by the formula (XVa): 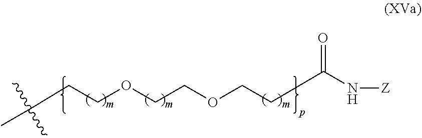
wherein
Z is the attachment group between the linker and the lipid anchor and is —C₁-C₁₀ alkyl-, —C₂-C₁₀ alkenyl-, —C₂-C₁₀ alkynyl-, —C₁-C₁₀ alkylC(O)—, —C₂-C₁₀ alkenylC(O)— or —C₂-C₁₀ alkynylC(O)—; or
Z, together with the adjacent amine, is an optionally C-terminal amidated amino acid selected from aspartic acid, glutamic acid, asparagine, glutamine, histidine, cysteine, lysine, arginine, serine or threonine; wherein the amino acid is attached to the lipid anchor via its side-chain functional group;
m is 1 or 2;
n is from 1 to 20; and
p is from 1 to 8.

9. A compound or pharmaceutically acceptable salt thereof selected from:

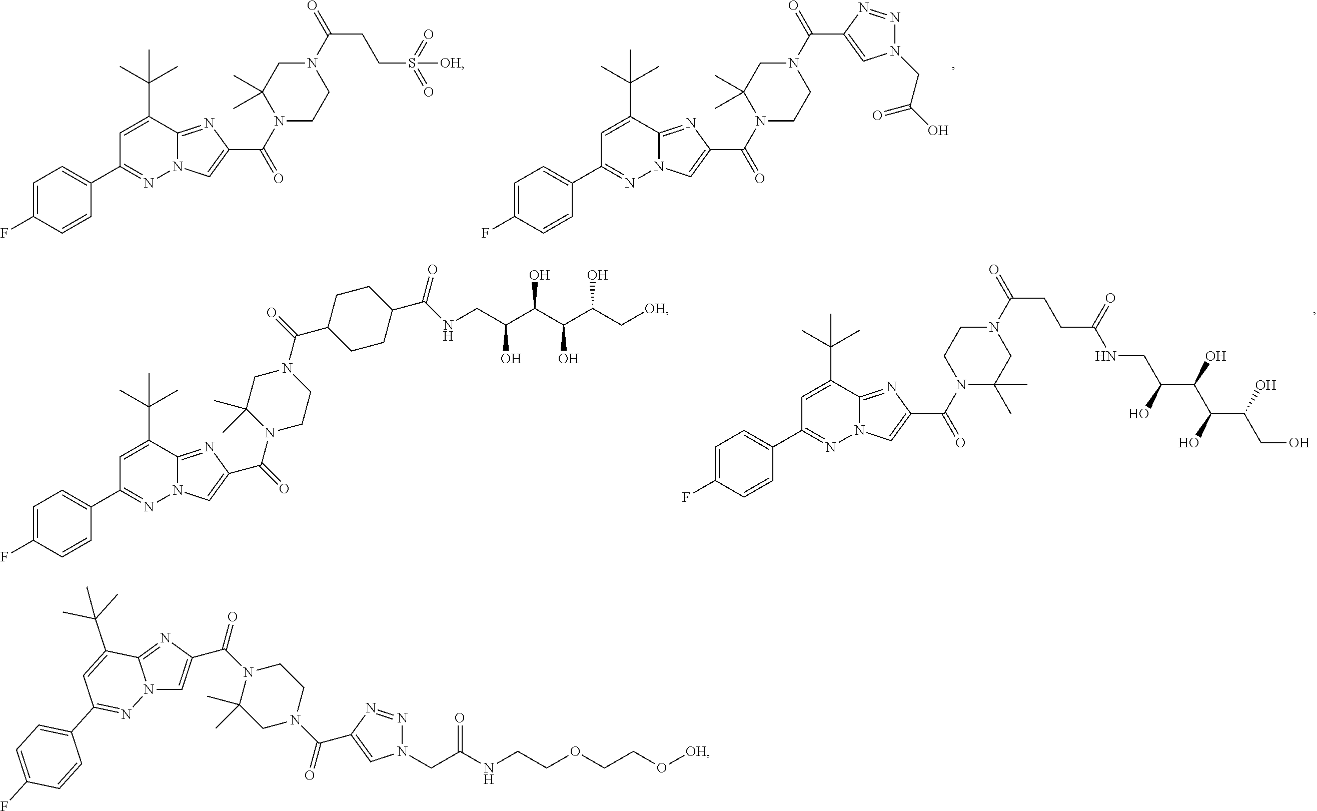

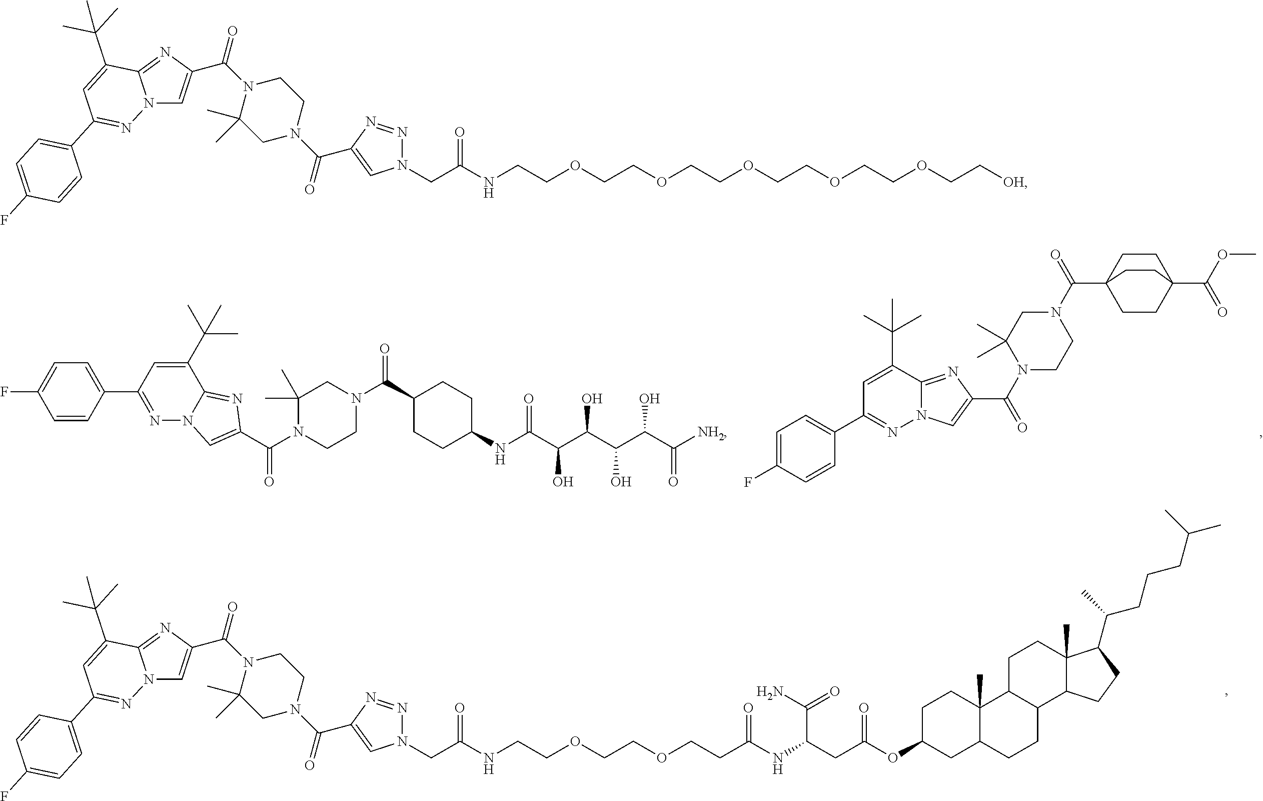

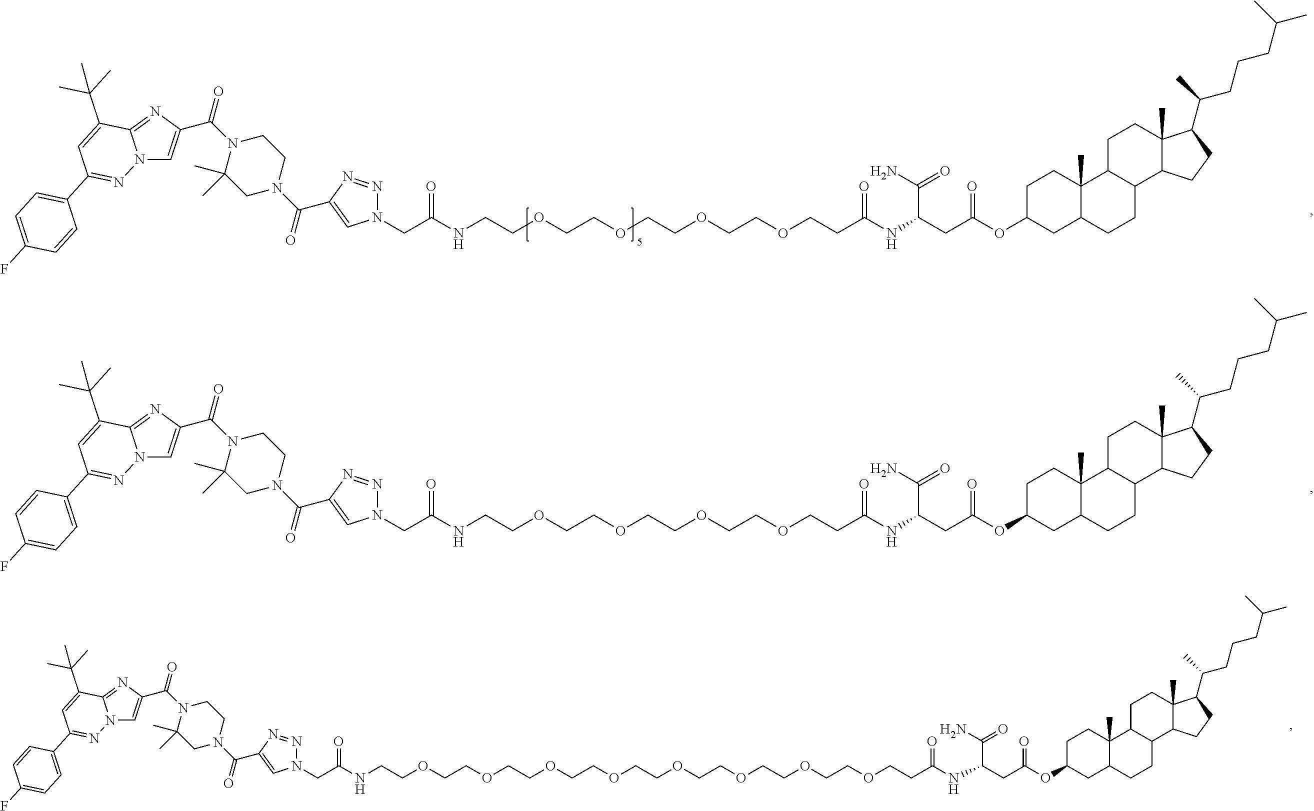

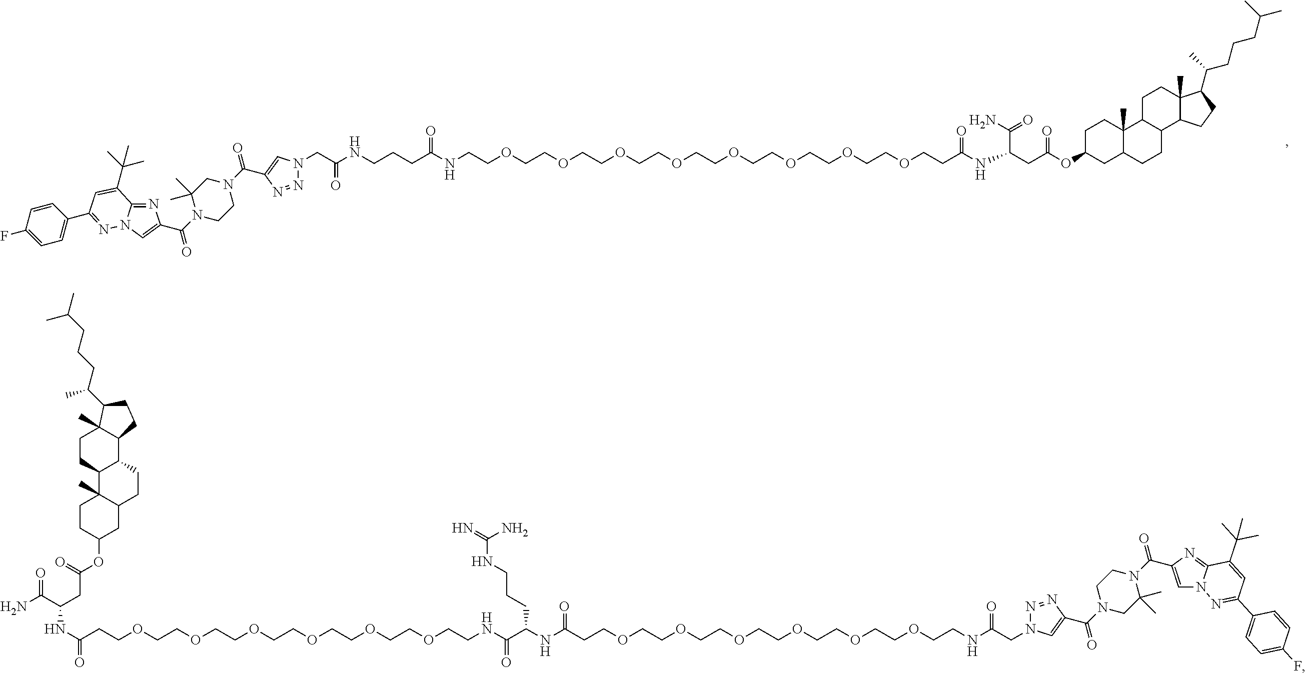

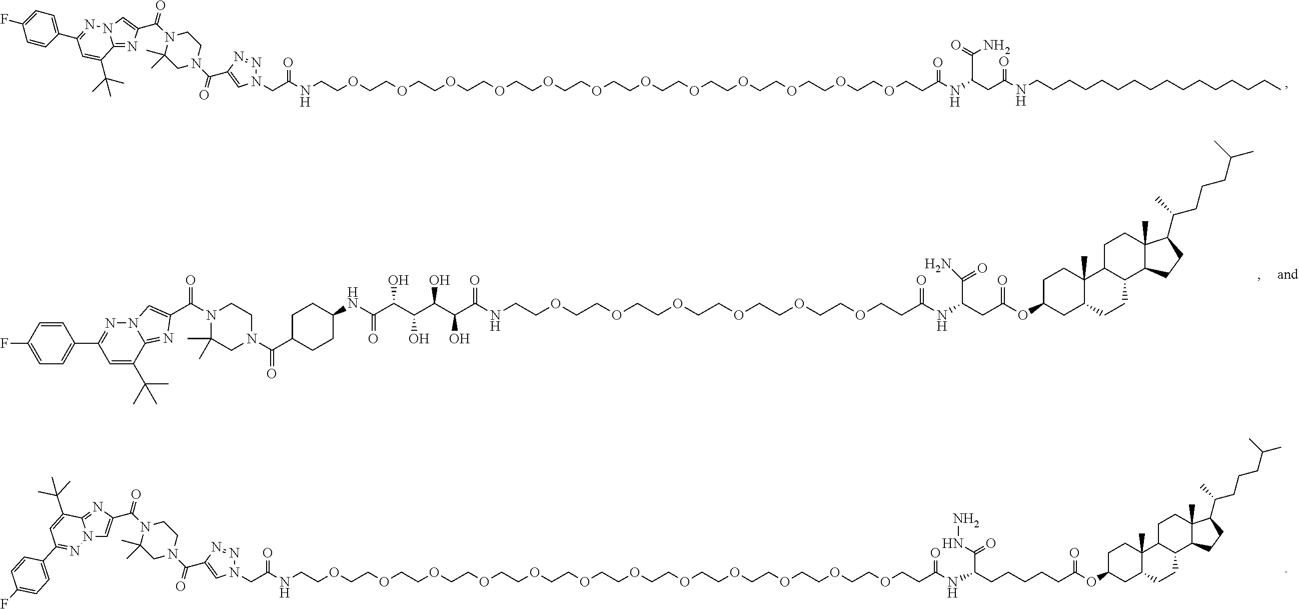

10. A pharmaceutical composition comprising a compound according to any one of claims 1 to 9 or a pharmaceutically acceptable salt thereof, together with at least one pharmaceutically acceptable carrier or diluent.

11. A method of inhibiting PAR₂ signaling comprising contacting the receptor with a compound according to any one of claims 1 to 9 or a pharmaceutically acceptable salt thereof.

12. A method of inhibiting PAR₂ signaling in a subject in need thereof, comprising administering to the subject an effective amount of a compound according to any one of claims 1 to 9 or a pharmaceutically acceptable salt thereof.

13. A method for preventing or treating a disease or disorder mediated by PAR₂ signaling, comprising administering to a subject in need thereof an effective amount of a compound according to any one of claims 1 to 9 or a pharmaceutically acceptable salt thereof.

14. The method according to claim 13, wherein the disease or disorder is mediated by endosomal PAR₂ signaling.

15. The method according to claim 13 or 14, wherein the disease or disorder mediated by PAR₂ signaling is selected from acute and chronic inflammatory disorders, tumour metastasis, gastrointestinal motility, pain, itch, skin disorders such as topic dermatitis, diet-induced obesity, asthma, rheumatoid arthritis, periodontitis, inflammatory bowel diseases, irritable bowel syndrome, cancer, fibrotic diseases, metabolic dysfunction, and neurological disease.

16. The method according to claim 13 or 14, wherein the disease or disorder mediated by PAR₂ signaling is pain associated with irritable bowel syndrome.

17. A compound according to any one of claims 1 to 9, or a pharmaceutically acceptable salt thereof, for the prophylaxis or treatment of a disease or disorder mediated by PAR₂ signaling.

18. Use of a compound according to any one of claims 1 to 9, or a pharmaceutically acceptable salt thereof, in the manufacture of a medicament for the prophylaxis or treatment of a disease or disorder mediated by PAR₂ signaling.

19. A compound according to any one of claims 1 to 9 or a pharmaceutical composition according to claim 10 for use in the prophylaxis or treatment of a disease or disorder mediated by PAR₂ signaling.

20. A compound according to any one of claims 1 to 9 or a pharmaceutical composition according to claim 10 for use in the prophylaxis or treatment of a disease or disorder selected from acute and chronic inflammatory disorders, tumour metastasis, gastrointestinal motility, pain, itch, skin disorders such as topic dermatitis, diet-induced obesity, asthma, rheumatoid arthritis, periodontitis, inflammatory bowel diseases, irritable bowel syndrome, cancer, fibrotic diseases, metabolic dysfunction, and neurological disease.
